(19) United States
(12) Patent Application Publication
(10) Pub. No.: US 2019/0125969 A1
(43) Pub. Date: May 2, 2019

(54) REMOTE ANALYTE MONITORING AND INSULIN DELIVERY SYSTEM

(71) Applicant: Senseonics, Incorporated, Germantown, MD (US)

(72) Inventors: Barbara Montgomery, Gaithersburg, MD (US); Andrew DeHennis, Germantown, MD (US)

(73) Assignee: Senseonics, Incorporated, Germantown, MD (US)

(21) Appl. No.: 16/177,596

(22) Filed: Nov. 1, 2018

Related U.S. Application Data
(60) Provisional application No. 62/580,348, filed on Nov. 1, 2017.
(60) Provisional application No. 62/580,798, filed on Nov. 2, 2017.

(51) Int. Cl. A61M 5/172

(52) U.S. Cl. CPC A61M 5/1723; A61M 2005/1726; A61M 2205/502; A61M 2205/3569; A61M 2205/3379; A61M 2205/584; A61M 2205/3313; A61M 2205/3368; A61M 2230/201; A61M 2205/8206; A61M 2205/18

(57) ABSTRACT

In one aspect, an analyte monitoring and insulin delivery system is provided. The system may include an analyte sensor configured to convey sensor data indicative of a measurement of one or more detectable properties based on an amount or concentration of an analyte. The system may include a transceiver configured to receive the sensor data and calculate an analyte level based on the sensor data. The system may include a display device configured to receive calculate an adjusted insulin delivery rate for an insulin pump and display the received analyte level and/or the adjusted insulin delivery rate. The system may include the insulin pump configured to increase, decrease or maintain a first insulin delivery rate based on the adjusted insulin delivery rate. The display device may further display icons corresponding to the system components and an operational status indicator for each icon.

### CROSS-REFERENCE TO RELATED APPLICATIONS

The present application claims the benefit of priority to U.S. Provisional Application Ser. No. 62/580,348, filed on Nov. 1, 2017, and U.S. Provisional Application Ser. No. 62/580,798, filed on Nov. 2, 2017, which are incorporated herein by reference in their entireties.

### BACKGROUND

### Field of Disclosure

This disclosure relates to an improved analyte monitoring and insulin delivery system (e.g., an improved artificial pancreas system).

### Discussion of Background

Known methods of artificial pancreas systems may include analyte monitoring and sensor augmented insulin pumps. Analyte monitoring may be used to monitor analyte levels, such as analyte concentrations. One type of analyte monitoring system is a continuous glucose monitoring (CGM) system. A CGM system measures glucose levels throughout the day and can be very useful in the management of diabetes. A conventional sensor augmented insulin pump may combine the technology of a standard insulin pump with the CGM system. In some embodiments, the sensor segmented insulin pump may be configured to receive the CGM system measured glucose levels and provide the received measurements to the user wearing the sensor augmented insulin pump. Accordingly, a user may personally administer insulin via the sensor augmented insulin pump based on the CGM system measured glucose levels.

The known methods of artificial pancreas systems, however, do not provide automatic dispensing of insulin by the sensor augmented insulin pump based on the CGM system measured glucose levels. Accordingly, there is a need for an improved analyte monitoring and insulin delivery system.

### SUMMARY

Aspects of the present invention relate to an improved analyte monitoring and insulin delivery system. In some embodiments, the analyte monitoring and insulin delivery system may provide automatic dispensing of insulin by an insulin pump based on analyte levels measured by an analyte monitoring system. In some embodiments, the system may additionally or alternatively show the operational status of one or more devices comprising the analyte monitoring and insulin delivery system. In some embodiments, the analyte monitoring and insulin delivery system may include one or more of an analyte monitoring sensor, an external transceiver, and an insulin pump. In some embodiments, the system may include a user interface configured to notify the user whether one or more devices of the system are functional and operating correctly.

One aspect of the invention may provide an analyte monitoring and insulin delivery system. The system may include an analyte sensor, a transceiver, an insulin pump, and a display device. The analyte sensor may include an analyte indicator that produces one or more detectable properties based on an amount or concentration of an analyte in proximity to the analyte indicator. The analyte sensor may be configured to convey sensor data indicative of a measurement of one or more of the detectable properties. The transceiver may be configured to receive the sensor data conveyed from the analyte sensor. The transceiver may be configured to calculate an analyte level using at least the received sensor data. The transceiver may be configured to convey the analyte level. The display device may be configured to receive the analyte level conveyed from the transceiver. The display device may be configured to calculate, based at least on the received analyte level, an adjusted delivery rate for the insulin pump. The display device may be configured to convey the adjusted delivery rate to the insulin pump. The display device may be configured to display, via a user interface, a first display including the received analyte level and the adjusted delivery rate. The display device may be configured to display, via the user interface, a second display including: (i) a sensor icon and a sensor status indicator indicating an operational status of the analyte sensor, (ii) a transceiver icon and a transceiver status indicator indicating an operational status of the transceiver, and (iii) a pump icon and an insulin pump status indicator indicating an operational status of the insulin pump. The insulin pump may be configured to deliver insulin at a first delivery rate. The insulin pump may be configured to receive the adjusted delivery rate conveyed by the display device. The insulin pump may be configured to increase, decrease or maintain the first delivery rate based on the received adjusted delivery rate.

In some embodiments, the analyte monitoring and insulin delivery system may further include an analyte meter. The analyte meter may be configured to receive the analyte level conveyed from the transceiver. The analyte meter may be configured to display the received analyte level. The second display may further include a meter icon and a meter status indicator indicating an operational status of the analyte meter.

In some embodiments, the display device may be further configured to compare the adjusted delivery rate with the first delivery rate. In some embodiments, the first display may further comprise a delivery rate indication of whether the adjusted delivery rate is an increase, a decrease, or a continuation of the first delivery rate.

In some embodiments, the display device may be further configured to compare the received analyte level with one or more previously stored analyte levels. In some embodiments, the first display may further comprise an indication of whether the received analyte level is an increase, a decrease or a continuation of the one or more previously stored analyte levels.

In some embodiments, the first display may further include an indication that the received analyte level is below, within, or above a predetermined range of analyte levels. In some embodiments, the first display may further include an indication that the received analyte level is below, within, or above a predetermined range of analyte levels. In some embodiments, the first display may further include an analyte trend graph icon, the display device may be further configured to display a third display in response to a user selection of the of the analyte trend graph icon, and the third display may include an analyte trend graph showing an analyte level line that indicates a change in analyte level over a period of time.

In some embodiments, the first display may further include an events panel comprising one or more event icons. In some embodiments, the second display may further include an autopilot icon and an autopilot status indicator indicating an operational status of automatic adjustment of insulin delivery. In some embodiments, the sensor, transceiver, and insulin pump status indicators may indicate an operational status with one of a normal sign, a warning sign, a failure sign, and a status unknown sign. In some embodiments, the second display may include a visual map of the analyte monitoring and insulin delivery system, and the visual map may include at least the sensor icon, the sensor status indicator, the transceiver icon, the transceiver status indicator, the pump icon, and the insulin pump status indicator. In some embodiments, one or more of the sensor icon, the transceiver icon, and the pump icon of the second display may be selectable, and the second device may be further configured to, in response to a selection of one of the icons, provide additional information about the one of the analyte sensor, the transceiver, and the insulin pump to which the selected icon corresponds.

Another aspect of the invention may provide an analyte monitoring and insulin delivery method. The method may include using a transceiver to receive sensor data from an analyte sensor. The analyte sensor may include an analyte indicator that produces one or more detectable properties based on an amount or concentration of an analyte in proximity to the analyte indicator. The analyte sensor may be configured to convey the sensor data. The sensor data may be indicative of a measurement of one or more of the detectable properties. The method may include using the transceiver to calculate an analyte level using at least the received sensor data. The method may include using the transceiver to convey the analyte level. The method may include using a display device to receive the analyte level conveyed from the transceiver. The method may include using the display device to calculate, based at least on the received analyte level, an adjusted delivery rate for an insulin pump. The method may include using the display device to convey the adjusted delivery rate to the insulin pump. The method may include using the display device to display, via a user interface, a first display comprising the received analyte level and the adjusted delivery rate. The method may include using the display device to display, via the user interface, a second display comprising: (i) a sensor icon and a sensor status indicator indicating an operational status of the analyte sensor, (ii) a transceiver icon and a transceiver status indicator indicating an operational status of the transceiver, and (iii) a pump icon and an insulin pump status indicator indicating an operational status of the insulin pump. The method may include using the insulin pump to deliver insulin at a first delivery rate. The method may include using the insulin pump to receive the adjusted delivery rate conveyed by the display device. The method may include using the insulin pump to increase, decrease, or maintain the first delivery rate based on the received adjusted delivery rate.

In some embodiments, the method may further include: using an analyte meter to receive the analyte level conveyed from the transceiver, and using the analyte meter display the received analyte level, and the second display may further include a meter icon and a meter status indicator indicating an operational status of the analyte meter. In some embodiments, the method may further include using the display device to compare the adjusted delivery rate with the first delivery rate, and the first display may further include a delivery rate indication of whether the adjusted delivery rate is an increase, a decrease, or a continuation of the first delivery rate. In some embodiments, the method may further include using the display device to compare the received analyte level with one or more previously stored analyte levels, and the first display may further include an indication of whether the received analyte level is an increase, a decrease or a continuation of the one or more previously stored analyte levels.

In some embodiments, the first display may further include an indication that the received analyte level is below, within, or above a predetermined range of analyte levels. In some embodiments, the first display may further include a message bar configured to display one or more messages containing information regarding the analyte monitoring and insulin delivery system. In some embodiments, the first display may further include an analyte trend graph icon, the method may further include displaying a third display in response to a user selection of the of the analyte trend graph icon, and the third display may include an analyte trend graph showing an analyte level line that indicates a change in analyte level over a period of time.

In some embodiments, the first display may further include an events panel comprising one or more event icons. In some embodiments, the second display may further include an autopilot icon and an autopilot status indicator indicating an operational status of automatic adjustment of insulin delivery. In some embodiments, the sensor, transceiver, and insulin pump status indicators may indicate an operational status with one of a normal sign, a warning sign, a failure sign, and a status unknown sign. In some embodiments, the second display may include a visual map of an analyte monitoring and insulin delivery system including at least the analyte sensor, transceiver, and insulin pump, and the visual map may include at least the sensor icon, the sensor status indicator, the transceiver icon, the transceiver status indicator, the pump icon, and the insulin pump status indicator. In some embodiments, one or more of the sensor icon, the transceiver icon, and the pump icon of the second display may be selectable, and the method may further include, in response to a selection of one of the icons, providing additional information about the one of the analyte sensor, the transceiver, and the insulin pump to which the selected icon corresponds.

Other features and characteristics of the subject matter of this disclosure, as well as the methods of operation, functions of related elements of structure and the combination of parts, and economies of manufacture, will become more apparent upon consideration of the following description and the appended claims with reference to the accompanying drawings, all of which form a part of this specification, wherein like reference numerals designate corresponding parts in the various figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated herein and form part of the specification, illustrate various embodiments of the subject matter of this disclosure. In the drawings, like reference numbers indicate identical or functionally similar elements.

FIG. 1 is a block diagram illustrating an analyte monitoring and insulin delivery system embodying aspects of the present invention.

FIG. 2 is a block diagram illustrating a processing system of a device embodying aspects of the present invention.

FIG. 3 is a block diagram illustrating a processing system of a device embodying aspects of the present invention.

FIGS. 4A-4C show various example system status screens embodying aspects of the present invention.

FIG. 5 shows various status indications embodying aspects of the present invention.

FIGS. 6A and 6B show various example system status screens embodying aspects of the present invention.

FIGS. 7A and 7B show various example system status screens embodying aspects of the present invention.

FIGS. 8-11 show various example system status screens embodying aspects of the present invention.

FIG. 12 shows an example dashboard screen embodying aspects of the present invention.

FIGS. 13A and 13B show a message bar in the dashboard screen embodying aspects of the present invention.

FIG. 14 shows a title bar in the dashboard screen embodying aspects of the present invention.

FIG. 15 shows a status bar in the dashboard screen embodying aspects of the present invention.

FIG. 16 shows a CGM panel and an insulin delivery panel in the dashboard screen embodying aspects of the present invention.

FIGS. 17A-17G show various example CGM panels in the dashboard screen embodying aspects of the present invention.

FIGS. 18A and 18B show various example insulin delivery panels in the dashboard screen embodying aspects of the present invention.

FIGS. 19A-19F show various example event panels in the dashboard screen embodying aspects of the present invention.

FIGS. 20-24 show various example dashboard screens embodying aspects of the present invention.

FIG. 25 shows an example add events screen embodying aspects of the present invention.

FIGS. 26A and 26B show an example of screens for saving a blood glucose embodying aspects of the present invention.

FIGS. 27A and 27B show an example of screens for blood glucose calibration embodying aspects of the present invention.

FIGS. 28A and 28B show an example of screens for blood glucose correction embodying aspects of the present invention.

FIGS. 29A and 29B show an example of screens for correction glucose cancellation embodying aspects of the present invention.

FIGS. 30A and 30B show an example of screens for blood glucose calibration and correction embodying aspects of the present invention.

FIG. 31 shows an example of screens for prompting a user to enter blood glucose embodying aspects of the present invention.

FIGS. 32A and 32B show an example of screens for delivering a meal bolus embodying aspects of the present invention.

FIGS. 33A and 33B show an example of screens for delivering an extended bolus embodying aspects of the present invention.

FIGS. 34A and 34B show an example of screens for cancelling an extended bolus embodying aspects of the present invention.

FIGS. 34C and 34D show an example of screens for delivering a hypo treatment embodying aspects of the present invention.

FIGS. 35A and 35B show an example of screens for adding an insulin event embodying aspects of the present invention.

FIGS. 36A and 36B show an example of screens for establishing an exercise target embodying aspects of the present invention.

FIGS. 37A and 37B show an example of screens for cancelling an exercise target embodying aspects of the present invention.

FIGS. 38A and 38B show an example of screens for establishing a sleep target embodying aspects of the present invention.

FIGS. 39A and 39B show an example of screens for cancelling a sleep target embodying aspects of the present invention.

FIG. 40 shows an example trend graph screen embodying aspects of the present invention.

FIG. 41 shows a title bar embodying aspects of the present invention.

FIGS. 42A-42C show calendar display embodying aspects of the present invention.

FIG. 43 shows a navigation bar embodying aspects of the present invention.

FIGS. 44A-44C show an axis embodying aspects of the present invention.

FIGS. 45A and 45B show target ranges embodying aspects of the present invention.

FIG. 46 shows time markers embodying aspects of the present invention.

FIGS. 47A-47E show a glucose line embodying aspects of the present invention.

FIG. 48 shows a basal line embodying aspects of the present invention.

FIGS. 49A-49D show event indications embodying aspects of the present invention.

FIGS. 50A and 50B show exemplary event icons embodying aspects of the present invention.

FIGS. 51 and 52 show pop over layers embodying aspects of the present invention.

FIGS. 53A-53C show dialog windows embodying aspects of the present invention.

### DETAILED DESCRIPTION

The Analyte Monitoring and Insulin Delivery System

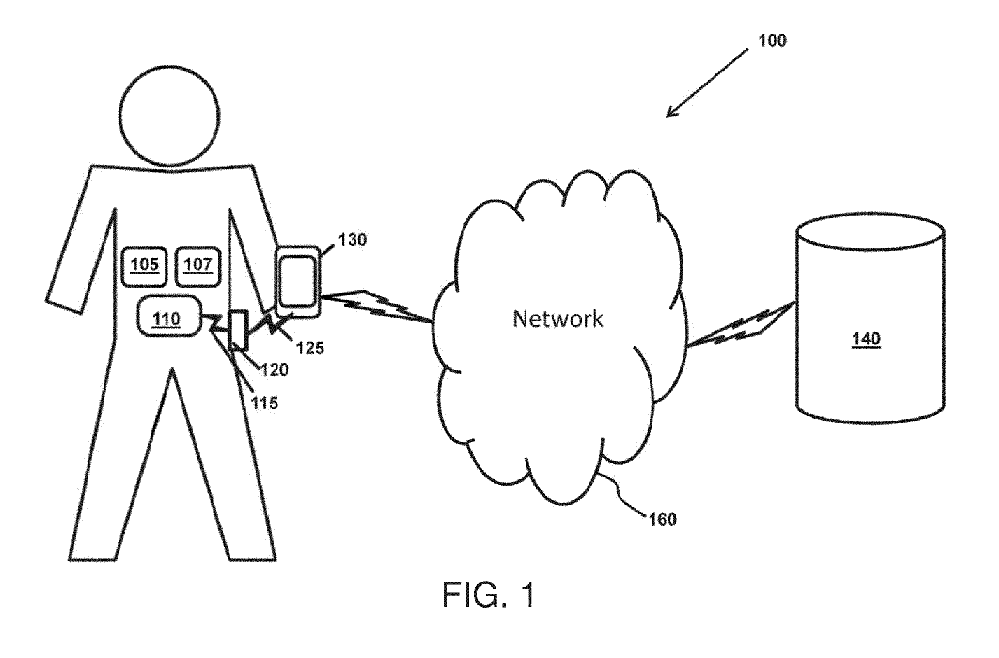

FIG. 1 is a block diagram of an analyte monitoring and insulin delivery system 100 illustrating connectivity between a plurality of devices that are configured to obtain sensor-measured analyte information (e.g., analyte levels) from an analyte monitoring sensor and utilize the obtained sensor-measured analyte information for insulin delivery according to one non-limiting embodiment of the present disclosure. In some embodiments, as shown in FIG. 1, the analyte monitoring and insulin delivery system 100 may include one or more of an insulin pump 105, an analyte meter 107, an analyte monitoring sensor 110, transceiver 120, a primary display device 130 and a data management system (DMS) 140. In some embodiments, the primary display device 130 and the DMS 140 may be in communication with each other via a network 160. In some embodiments, the analyte monitoring sensor 110 may be in communication with the transceiver 120 over a first communication link 115. In some embodiments, the first communication link 115 may be a wireless communication link or a wired communication link. Examples of wired communication links comprise, but are not limited to: cable, wire, twisted-pair wire, fiber-optic, Ethernet, USB, and/or the like. Examples of wireless communications links comprise, but are not limited to: cellular, Wi-Fi, Bluetooth™, Near-Field Communications (NFC), infrared, radar, satellite, radio frequency, combinations thereof, and/or the like. In some embodiments, the transceiver 120 may be in communication with the primary display device 130 by a second communication link 125. In one embodiment, the second communication link 125 may be a wireless link, such as Bluetooth™

In some embodiments, the primary device 130 and the DMS 140 may be in communication with each other via the network 160. In some embodiments, the primary device 130 may be configured to transmit sensor-measured analyte information to the DMS 140. The network 160 may comprise, but is not limited to: local area network (LAN), wide area network (WAN), the Internet, intranets, cellular, combinations thereof, and/or the like.

In some embodiments, the analyte monitoring sensor 110 may be, for example and without limitation, a wireless, implantable sensor inserted subcutaneously inside a patient. In some embodiments, the analyte monitoring sensor 110 may be configured to measure interstitial fluid glucose levels of the patient. In some embodiments, the analyte monitoring sensor 110 may comprise, for example, a fluorometer. A fluorometer or fluorimeter may comprise a device (e.g., a photodetector) configured to measure parameters of fluorescence such as, for example, its intensity and/or wavelength distribution of emission spectrum after and/or during excitation by a spectrum of light. These parameters may be employed to identify the presence and/or the amount of specific molecules in a medium.

In some non-limiting embodiments, the analyte sensor 110 may be encased in a sensor housing (i.e., body, shell, capsule, or encasement), which may be rigid and biocompatible. The sensor 110 may include an analyte indicator, such as, for example, a polymer graft coated, diffused, adhered, or embedded on or in at least a portion of the exterior surface of the sensor housing. The analyte indicator (e.g., polymer graft) of the sensor 110 may include indicator molecules (e.g., fluorescent indicator molecules) that produce (e.g., exhibit) one or more detectable properties (e.g., optical properties) based on the amount or concentration of the analyte in proximity to the analyte indicator. In some embodiments, the sensor 110 may include a light source that emits excitation light over a range of wavelengths that interact with the indicator molecules. The sensor 110 may also include one or more photodetectors (e.g., photodiodes, phototransistors, photoresistors, or other photosensitive elements). One or more of the photodetectors may be sensitive to emission light (e.g., fluorescent light) emitted by the indicator molecules such that a signal generated by a photodetector in response thereto that is indicative of the level of emission light of the indicator molecules and, thus, the amount of analyte of interest (e.g., glucose). In some non-limiting embodiments, one or more of the photodetectors may be covered by one or more filters that allow only a certain subset of wavelengths of light to pass through (e.g., a subset of wavelengths corresponding to the emission light) and reflect the remaining wavelengths. In some non-limiting embodiments, the sensor 110 may include a temperature transducer.

In some embodiments, the analyte monitoring sensor 110 may be configured to communicate sensor data to the transceiver 120 over either a wired communication link or a wireless communication link. In some embodiments, the sensor data may include information indicative of one or more measurements of one or more the detectable properties of the analyte indicator. In some embodiments, the sensor data may include information indicative of the temperature of the sensor 110. In some embodiments, the transceiver 120 may be configured to receive the sensor data from the analyte monitoring sensor.

In some embodiments, the transceiver 120 may additionally be configured to power the analyte monitoring sensor via wireless power transfer mechanisms. In some non-limiting embodiments, the transceiver 120 may be connected to a power source, such as a battery, a transformer, a power line, or the like. In some embodiments, the transceiver 120 may transmit power by electromagnetic fields across an intervening space to one or more receiver devices. In some embodiments, the analyte monitoring sensor 110 may connect the electromagnetic fields back to electric power and utilize the power. In some embodiments, the transceiver 120 may be a reusable device disposed external to the patient at a location within communications range of analyte monitoring sensor 110. Depending upon the power capabilities of transceiver 120, the location of transceiver 120 may be on or near the skin of patient and/or at a distance from patient.

In some embodiments, the transceiver 120 may be configured to calculate one or more analyte levels based on sensor data received from the analyte sensor 110. In some embodiments, the calculated analyte levels may be additionally based on one or more reference measurements. The reference measurements may be, for example and without limitation, self-monitoring blood glucose (SHBG) measurements. The reference measurements may be, for example and without limitation, obtained from finger-stick blood samples. In some non-limiting embodiments, the transceiver 120 may receive the one or more reference measurements from the primary display device 130.

In some embodiments, the transceiver 120 may be configured to communicate to one or more of the devices over one or more communications links. By communicating to one or more devices, the transceiver 120 may be configured to transmit the analyte information obtained from the analyte monitoring sensor 110 to the one or more devices of the analyte monitoring system 100, such as the insulin pump 105 and the analyte meter 107. In some embodiments, the analyte information may include one or more calculated analyte levels (e.g., a calculated blood glucose level). In an embodiment, the transceiver 120 may be configured to transmit the analyte information to the primary device 130 and/or the DMS 140.

In some embodiments, the insulin pump 105 may be in communication with one or more of the primary display device 130, the DMS 140, and the transceiver 120 over either a wired communication link or a wireless communication link. Examples of the wireless communication link may comprise, but is not limited to: cellular, Wi-Fi, BluetoothTM, Near-Field Communications (NFC), infrared, radar, satellite, radio frequency, combinations thereof, and/or the like. In some embodiments, the insulin pump 105 may be configured to receive the analyte information obtained from the analyte monitoring sensor 110 from at least one of the DMS 140, the primary device 130, and the transceiver 120. In some embodiments, the insulin pump 105 may be configured to automatically adjust the amount of insulin being delivered based on the received analyte information (e.g., based on one or more received analyte levels). In such embodiments, the insulin pump 105 may be configured to calculate and adjust the amount of insulin to be delivered by the insulin pump 105 based on the received analyte information. In some embodiments, the adjusted amount of insulin may be calculated based on proprietary insulin dosing algorithms stored in the insulin pump 105. In some embodiments, the insulin pump 105 may transmit the calculated amount of insulin to one or more of the primary device 130, the DMS 140, and the analyte meter 107.

In some alternative embodiments, the insulin pump 105 may be configured to receive a calculated amount of insulin to be delivered by the insulin pump 105 from one or more of the primary display device 130, the transceiver 120, and the DMS 140. In such embodiments, one or more of the primary display device 130, the transceiver 120, and the DMS 140 may be configured to calculate the amount of insulin to be delivered by the insulin pump 105 based on the received analyte information (e.g., based on one or more received analyte levels). In such embodiments, the adjusted amount of insulin may be calculated based on proprietary insulin dosing algorithms stored in one or more of the primary display device 130, the transceiver 120, and the DMS 140. In such embodiments, the insulin pump 105 may adjust the amount of insulin to be delivered based on the received calculated amount of insulin.

In some embodiments, the analyte meter 107 may be in communication with one or more of the primary device 130, the DMS 140, and the insulin pump 105 over either a wired communication link or a wireless communication link. Examples of the wireless communication link may comprise, but is not limited to: cellular, Wi-Fi, BluetoothTM, Near-Field Communications (NFC), infrared, radar, satellite, radio frequency, combinations thereof, and/or the like. In some embodiments, the analyte meter 107 may be configured to receive the analyte information obtained from the analyte monitoring sensor 110 from at least one of the DMS 140, the primary device 130, and the transceiver 120. In such embodiments, the analyte meter 107 may be configured to display the received analyte information (e.g., one or more analyte levels) to the user. In some embodiments, the analyte meter 107 may be configured to receive the calculated amount of insulin, e.g., the current rate and/or an adjustment in the rate in which the insulin is being delivered by the insulin pump 105, from one or more of the DMS 140, the primary display device 130, the transceiver 120, and the insulin pump 105. In such embodiments, the analyte meter 107 may be configured to display the current rate and/or the adjustment in the rate in which the insulin is being delivered by the insulin pump 105.

In some embodiments, the primary display device 130 of the analyte monitoring and insulin delivery system 100 may comprise a mobile display device such as, but not limited to: a smart phone, a tablet, an iPod, a health monitoring watch, and/or the like. However, in some alternative embodiments, the plurality of devices of the analyte monitoring system may comprise another type of display device such as, but not limited to: a personal computer, a netbook, a medical monitoring device, and/or the like. Furthermore, the primary device 130 may comprise a medical device, such as, but not limited to: a blood glucose meter, an insulin pump, a combination thereof, and/or the like. In some embodiments, the primary device 130 may be configured to receive monitored analyte information transmitted by the transceiver 120. The primary device 130 may store the received analyte information in memory for further processing. In such embodiments, the primary device 130 may be configured to calculate and adjust the amount of insulin to be delivered by the insulin pump 105 based on the received analyte information. In some embodiments, the adjusted amount of insulin may be calculated based on proprietary insulin dosing algorithms stored in the primary device 130. The calculated amount of insulin may be transmitted by the primary device 130 to the insulin pump 105. In some embodiments, the primary device 130 may transmit the calculated amount of insulin to one or more of the DMS 140 and the analyte meter 107. In some embodiments, the primary device 130 may additionally or alternatively transmit the received analyte information to one or more of the DMS 140, the insulin pump 105, and the analyte meter 107.

In some embodiments, the DMS 140 may be a server device employed to allow data to be shared over the network such as the Internet. The server may share data via proprietary formats configured to be employed by hardware computing systems configured, at least in part, with applications to make the hardware computing system into an analyte monitoring system. In some embodiments, the DMS 140 may be a web-based DMS (e.g., hosted on a remote server). In some embodiments, monitored analyte information transmitted by the primary device 130 and/or the transceiver 120 may be uploaded (e.g., through a wired connection such as, for example, a USB connection or a wireless connection such as, for example, a wireless Internet connection) to the DMS 140. In some embodiments, the DMS 140 may enable sharing of the analyte data (e.g., allowing the user, caregiver, and/or clinician to view sensor analyte data). In some embodiments, the DMS 140 may further process the received analyte information. In such embodiments, the DMS 140 may be configured to calculate and adjust the amount of insulin to be delivered by the insulin pump 105 based on the received analyte information. In some embodiments, the adjusted amount of insulin may be calculated based on proprietary insulin dosing algorithms stored in the DMS 140. The calculated amount of insulin to be delivered may be transmitted by the DMS 140 to the insulin pump 105. In some embodiments, the DMS 140 may transmit the calculated amount of insulin to one or more of the primary device 130 and the analyte meter 107. In some embodiments, the DMS 140 may additionally or alternatively transmit the received analyte information to one or more of the primary device 130, the insulin pump 105, and the analyte meter 107.

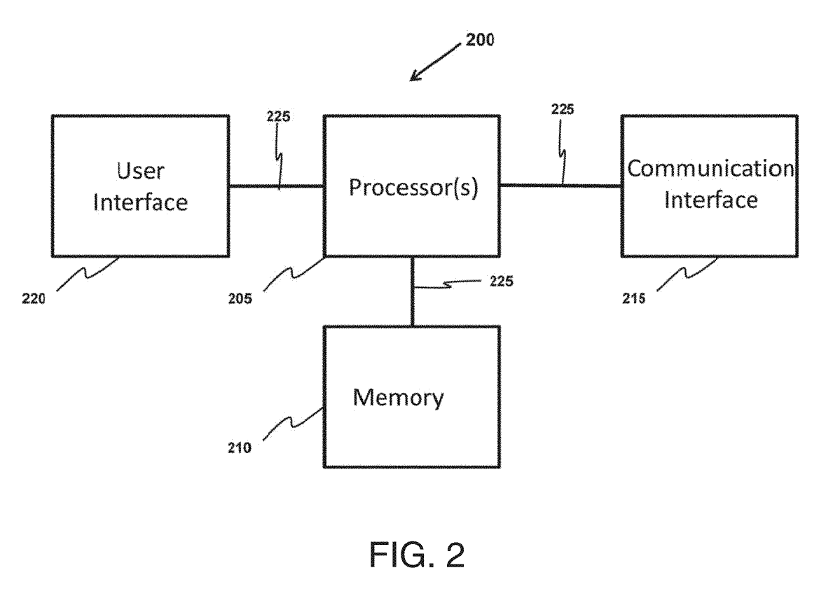

FIG. 2 illustrates a processing system 200 residing in a device of analyte monitoring system according to an embodiment of the present invention. In some embodiments, shown in FIG. 2, each device of the analyte monitoring system may include a processing system 200 consisting of one or more processors 205, a memory 210, a communication interface 215, a user interface 220, and/or a bus 225 that couples the various processing system components including the memory 210 to the one or more processors 205. In some embodiments, the one or more processors 205 may consist of one or more central processing units (CPUs) that execute computer program instructions stored in the memory 210 to perform functions described herein with respect to the one or more devices. These functions may be configured to improve the technological field of analyte monitoring and drug dispersion on a living patient. In some embodiments, the memory 210 may include computer storage media in the form of volatile and/or nonvolatile memory such as ROM and RAM. In some embodiments, the memory 210 may additionally or alternatively include non-removable nonvolatile computer storage media, such as, for example and without limitation, a hard disk drive or removable nonvolatile computer storage media that is configured to read from a flash drive, optical disk drive, or other optical media. In some embodiments, the drives and their associated computer storage media may provide storage of computer readable instructions, data structures, program modules and other data for the processing system, which are inputted to the one or more processors for the performance of particular tasks.

In some embodiments, the user interface 220 of the processing system 200 may enable an operator to control the device (e.g., primary device 130) by providing one or more input and/or output devices. The input and/or output devices may include, for example and without limitation, pushbutton(s), a keyboard, a microphone, a camera, a pointing device (e.g., a mouse, trackball, or touch pad), touch screen(s), voice interfaces(s), multimedia interface(s), audio interface(s), tactile interfaces(s), visual interface(s), monitor(s), combinations thereof, and/or the like. Accordingly, in some embodiments, a user may enter commands and information into the device through input devices, and the device may present the monitored analyte and insulin delivery information to the user via the output devices.

In some embodiments, the communication interface 215 of the processing system 200 may enable the device to be operated in a networked environment using logical connections to the analyte monitor sensor 110, transceiver 120, the insulin pump 105, the analyte meter 107, the DMS 140 and other devices of the analyte monitoring and insulin delivery system 100. In some embodiments, the communication interface 215 is configured to communicate directly with the analyte sensor 110. In some alternative embodiments, the communication interface 215 may be configured to additionally or alternatively relay analyte information obtained from the analyte sensor 110 to other devices of the analyte monitoring and insulin delivery system 100. In some embodiments, the one or more processor 205 may calculate and adjust the amount of insulin to be delivered by the insulin pump 105 based on analyte information obtained from devices of the analyte monitoring and insulin delivery system 100. In such embodiments, the adjusted amount of insulin may be calculated based on proprietary insulin dosing algorithms stored in the memory 210. The calculated amount of insulin may be transmitted via the communication interface 215 to the insulin pump 105. In some embodiments, the device may be connected, through the communication interface 215, to a remote application program residing on a remote processing device, in which the device and the remote processing device are linked via the network. Accordingly, the device may delegate particular tasks to the remote application program residing on the remote processing device.

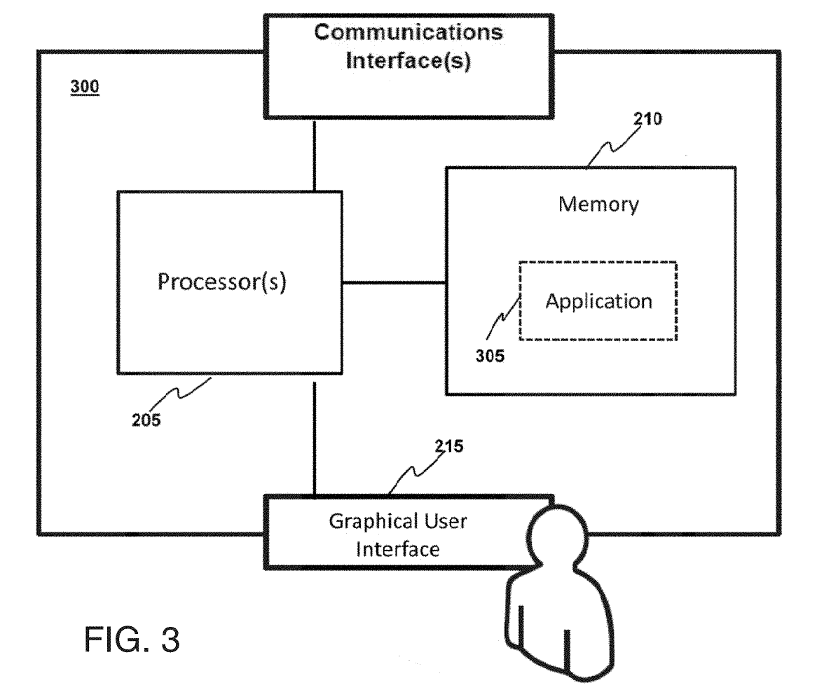

One or more mobile medical applications 305 (“MMA”) may be provided, for example, to execute in the plurality of devices of the analyte monitoring and insulin delivery system 100. Referring to FIG. 3, the MMA 305 is provided in the form of computer readable instructions stored in the memory 210 of a device 300 and is configured to be executed by the one or more processors 205 in the processing system of the device 300 according to an embodiment of the present invention. Where the device 300 is coupled to a display, the MMA 305 may cause the device 300 to provide a series of graphical control elements or widgets, such as a graphical user interface (GUI) 215, shown on the display. As a result, the MMA 305 may, for example, cause the device 300 to display analyte and delivered insulin related information in the GUI 215 such as, but not limited to: one or more of glucose information, current glucose readings, user notifications, glucose status alerts and alarms, trend graphs and arrows, and user-entered events, operational status of devices in the analyte monitoring and insulin delivery system 100, and may provide one or more graphical control elements that may allow a user to manipulate aspects of the one or more display screens. Although aspects of the application 305 are described in the context of glucose monitoring and insulin delivery system embodiments below, this is not required, and, in some alternative embodiments, the application 305 may be employed in other types of analyte monitoring and insulin delivery systems.

System Status Screen

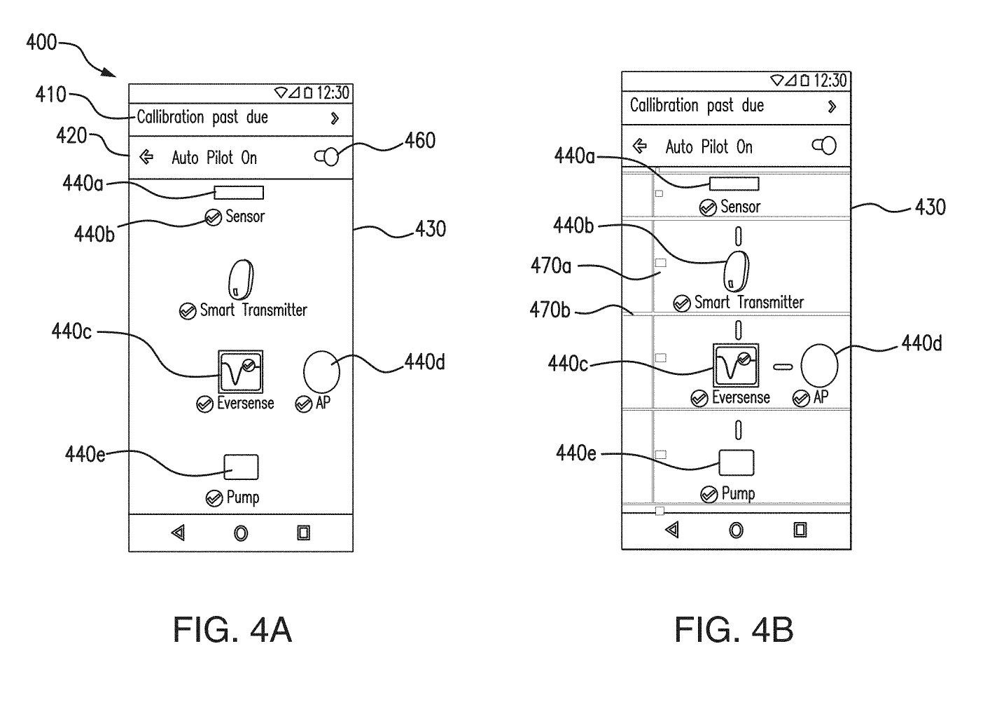

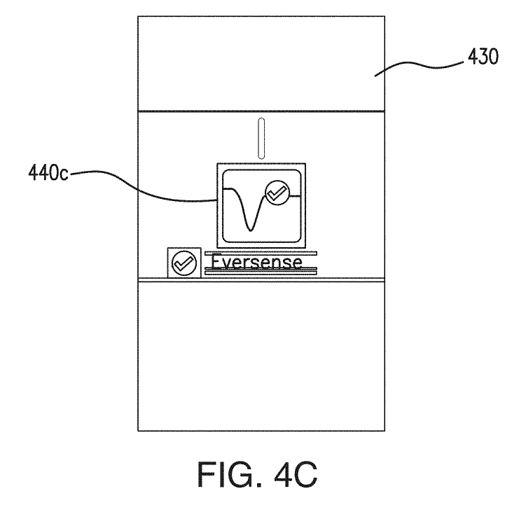

FIGS. 4A-4C show various example system status screens embodying aspects of the present invention. FIG. 4A shows an example system status screen 400 generated by the MMA 305 for display on the primary device 130 in accordance to an embodiment of the present invention. When a user gains access to his or her user account, the MMA 305 may configure the primary display device 130 to present the system status screen 400 on a display of the primary display device 130. As shown in FIG. 4A, the system status screen 400 may display one or more of a message bar 410, a title bar 420, and a visual map 430 showing one or more of the devices of the user's analyte monitoring and insulin delivery system 100, as described above, for instance, in FIG. 1. In some embodiments, the visual map 430 of the devices may comprise one or more of a sensor icon 440a, a smart transmitter icon 440b, an MMA 305 icon 440c, an autopilot (“AP”) icon 440d, and an insulin pump icon 440e. In such embodiments, the icons 440a-e may correspond to the devices of the analyte monitoring and insulin delivery system 100. In a non-limiting example, the sensor icon 440a may correspond to the analyte monitoring sensor 110, the smart transmitter icon 440b may correspond to the transceiver 120, and the pump icon 440e may correspond to the insulin pump 105. In some embodiments, the MMA icon 440c may correspond to the MMA 305 being executed by the primary device 130, and the AP icon 440d may correspond to an autopilot functionality of the analyte monitoring and insulin delivery system 100.

As shown in FIG. 4A, a line between each of the icons 440a-e may indicate the connection between the devices in the analyte monitoring and insulin delivery system 100. As a non-limiting example, FIG. 4A shows a line between the sensor icon 440a and the smart transmitter icon 440b, indicating that there is a wired and/or wireless connection between the corresponding analyte monitoring sensor 110 and the transceiver 120. In some embodiments, one or more of the icons 440a-e may be selectable (e.g., touchable or tappable) by the user. Upon detection of a selection of one of the icons 440a-e, the MMA 305 may configure the primary device 130 to present a screen comprising additional information regarding the device and/or function corresponding to the selected icon 440a-e. In a non-limiting example, if a user selects the smart transmitter icon 440b, the primary device 130 may present a screen to the user comprising information relating to, but not limited to, remaining battery for the transceiver 120, the devices and/or functions the transceiver 120 is connected to (e.g., analyte monitoring sensor 110 and MMA 305), the current operational status of the transceiver 120, among others. In some embodiments, upon detection of a selection of an icon 440a-e, the MMA 305 may configure the primary device 130 to present a screen comprising the selected icon. In a non-limiting example, if a user selects the MMA icon 440c, the primary device 130 may present a screen to the user comprising the selected MMA icon 440c as a single icon in the visual map 430, as shown in FIG. 4C. In some embodiments, the single icon, as shown in FIG. 4C, may be selected, thereby triggering the primary device 130 to present a screen comprising specific information (e.g., current operational status, etc.) regarding the device and/or function corresponding to the selected icon 440a-e.

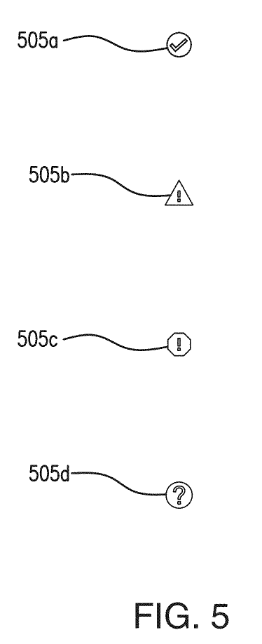

As shown in FIG. 4A, each icon 440a-e may include an icon name and a status indication 450 of the device corresponding to the icon 440a-e. The status indication 450 may provide an indication to the user whether or not each corresponding device or function in the analyte monitoring and insulin delivery system 100 is operational and functioning properly. As a non-limiting example, FIG. 4A shows the status indication 450 of the sensor icon 430 as a check mark, which indicates to the user that the analyte monitoring sensor 110 is operational and functioning properly. The status indication 450 is explained in more detail in the following FIG. 5.

In some embodiments, the message bar 410 may be configured to display system messages. In some embodiments, system messages may comprise messages regarding any information requiring the user's attention regarding the analyte monitoring and insulin delivery system 100. As a non-limiting example, the message bar 410 in FIG. 4A displays “Calibration past due.” Accordingly, the message bar 410 provides the user with a notification that the analyte monitoring and insulin delivery system 100 requires calibration. Other non-limiting examples of the message bar 410 are described in the following description.

In some embodiments, the title bar 420 comprises an autopilot (“AP”) switch 460. In some embodiments, the AP switch 460 may be configured to turn on/off AP functionality of the analyte monitoring and insulin delivery system 100. In some embodiments, the AP functionality relates to the automatic adjustment of insulin dispensing performed by the insulin pump 105 based on analyte levels monitored by the analyte monitoring sensor 110 and transmitted by the transceiver 120. As a non-limiting example, if the AP switch 460 is turned on, the insulin pump 405 may be configured to automatically calculate and adjust the amount of insulin to be delivered by the insulin pump 105 based on the received analyte information. In some embodiments, the adjusted amount of insulin may be calculated based on proprietary insulin dosing algorithms stored in the insulin pump 105. In some embodiments, the calculation may be performed by the primary device 130 and/or the DMS 140, the proprietary insulin dosing algorithms stored in the primary device 130 and/or the DMS 140. In such embodiments, the insulin pump 405 may be configured to receive the calculated amount of insulin to be delivered by the insulin pump 105 from one or more of the primary device 130 and the DMS 140 and adjust the amount of insulin to be delivered based on the received calculated amount of insulin. In another non-limiting example, if the AP switch is turned off, the automatic calculation and adjustment of the insulin delivered by the insulin pump 105 is deactivated and the insulin pump 105 may deliver scheduled basal insulin based on a predetermined configuration.

In some embodiments, the visual map 430 may comprise a set of repeated, stacked icons 440a-e, as shown in FIG. 4B. Vertical guideline 470a and horizontal guideline 470b (guidelines 470a-b are shown if FIG. 4B for the purposes of explanation and are not displayed to the user in the visual map 430) indicate the one or more stacks for each of the icons 440a-e, according to some embodiments.

FIG. 5 shows the various statuses 505a-d that may be shown by the status indication 450. As shown in FIG. 5, the status indication 450 may show a check surrounded by a green circle (“normal”) 505a, which indicates that a device is operational and functioning properly, according to some embodiments. In some embodiments, the status indication 450 may show an exclamation mark surrounded by a yellow triangle (“warning sign”) 505b, which indicates a warning regarding a device and that the user should check the device for proper operation. In some embodiments, the warning may be regarding a connection issue for the device. In some embodiments, the status indication 450 may show an exclamation mark surrounded by a red octagon (“failure sign”) 505c, which may indicate a device failure and that the user must replace and/or check the device for proper operation. In some embodiments, the status indication 450 may show a question mark surrounded by a circle (“status unknown sign”) 505d, which may indicate that a status of a device is unknown.

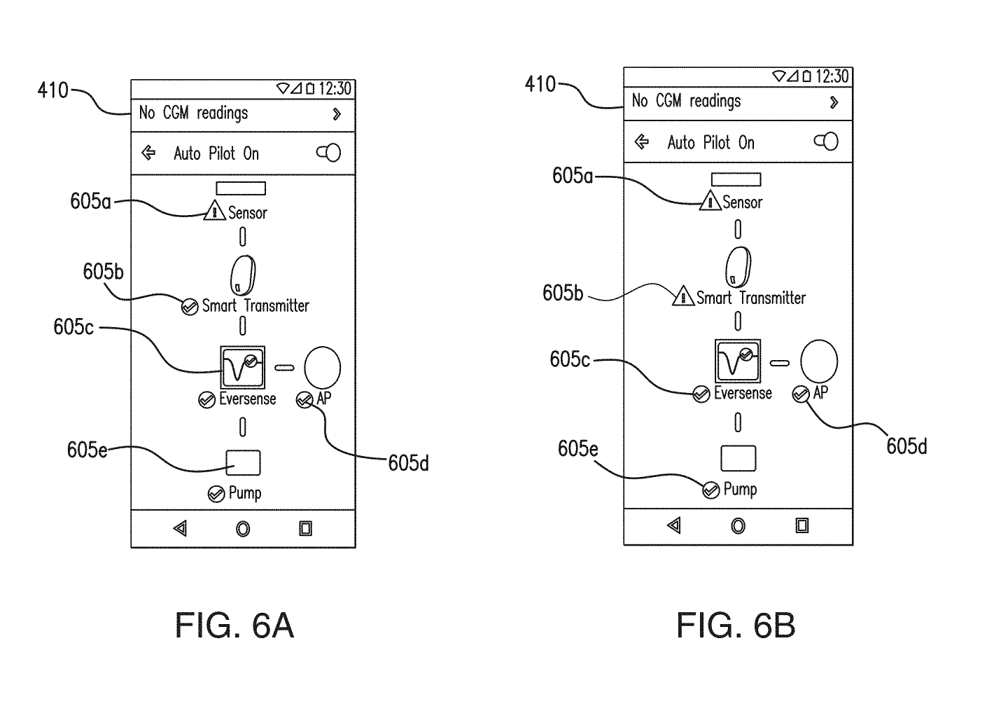

In addition to normal operation of the devices in the analyte monitoring and insulin delivery system 100, as shown by the system status screen 400 shown in FIG. 4A, there may be five additional states in the analyte monitoring and insulin delivery system 100, according to some embodiments. In some embodiments, a first additional state in the analyte monitoring and insulin delivery system 100 may comprise an analyte monitoring buffer state (e.g., CGM buffer), as shown in FIGS. 6A and 6B. In some embodiments, the analyte monitoring buffer state may be caused due to the analyte monitoring sensor 110 not being turned on and/or connected to the analyte monitoring and insulin delivery system 100. Accordingly, FIG. 6A shows a status indication 605a of the analyte monitoring sensor 110 showing a warning sign while the remaining status indications 605b-e show a normal sign. In some embodiments, the analyte monitoring buffer may be caused due to the analyte monitoring sensor 110 and/or the transceiver 120 not being turned on and/or connected to the analyte monitoring and insulin delivery system 100. Accordingly, FIG. 6B shows a status indication 605a of the analyte monitoring sensor 110 and a status indication 605b of the transceiver 120 showing a warning sign while the remaining status indications 605c-e show a normal sign. As shown in FIGS. 6A and 6B, the message bar 410 may display “No CGM readings” according to the analyte monitoring buffer state of the analyte monitoring and insulin delivery system 100.

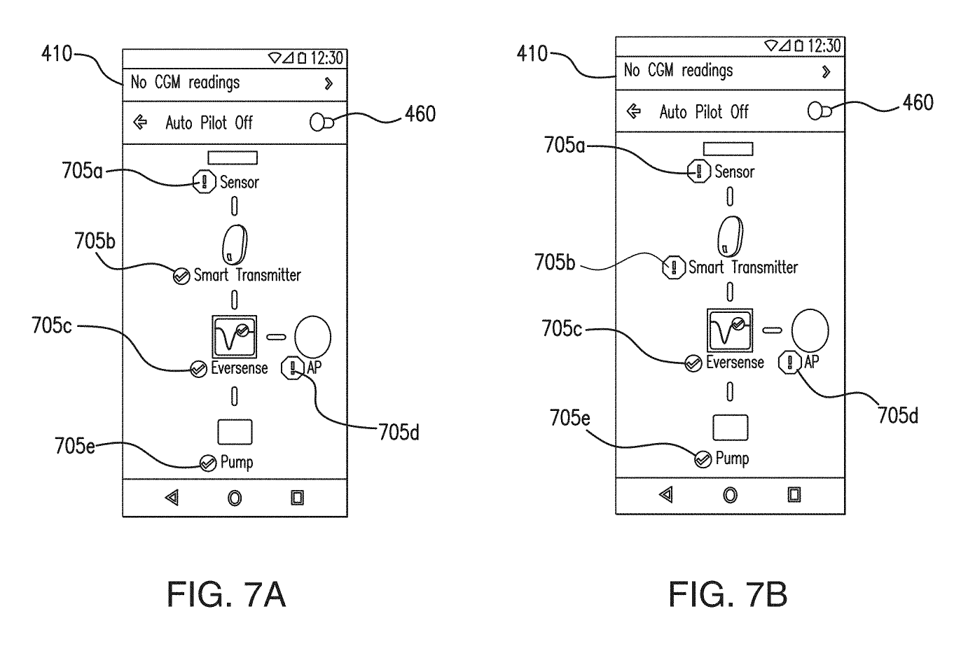

In some embodiments, a second additional state in the analyte monitoring and insulin delivery system 100 may comprise an analyte monitoring stopped state (e.g., CGM stopped), as shown in FIGS. 7A and 7B. In the non-limiting examples shown in FIGS. 7A and 7B, the AP switch 460 is turned off, which may cause the status indication 705d of the AP function to display a failure sign. In some embodiments, the analyte monitoring stopped state may be caused due to the analyte monitoring sensor 110 not being turned on and/or connected to the analyte monitoring and insulin delivery system 100. Accordingly, FIG. 7A shows a status indication 605a of the analyte monitoring sensor 110 showing a failure sign while the remaining status indications 605b, c, e show a normal sign. In some embodiments, the analyte monitoring stopped state may be caused due to the analyte monitoring sensor 110 and/or the transceiver 120 not being turned on and/or connected to the analyte monitoring and insulin delivery system 100. Accordingly, FIG. 7B shows a status indication 605a of the analyte monitoring sensor 110 and a status indication 605b of the transceiver 120 showing a failure sign while the remaining status indications 605c, e show a normal sign. As shown in FIGS. 7A and 7B, the message bar 410 may display “No CGM readings” according to the analyte monitoring stopped state of the analyte monitoring and insulin delivery system 100.

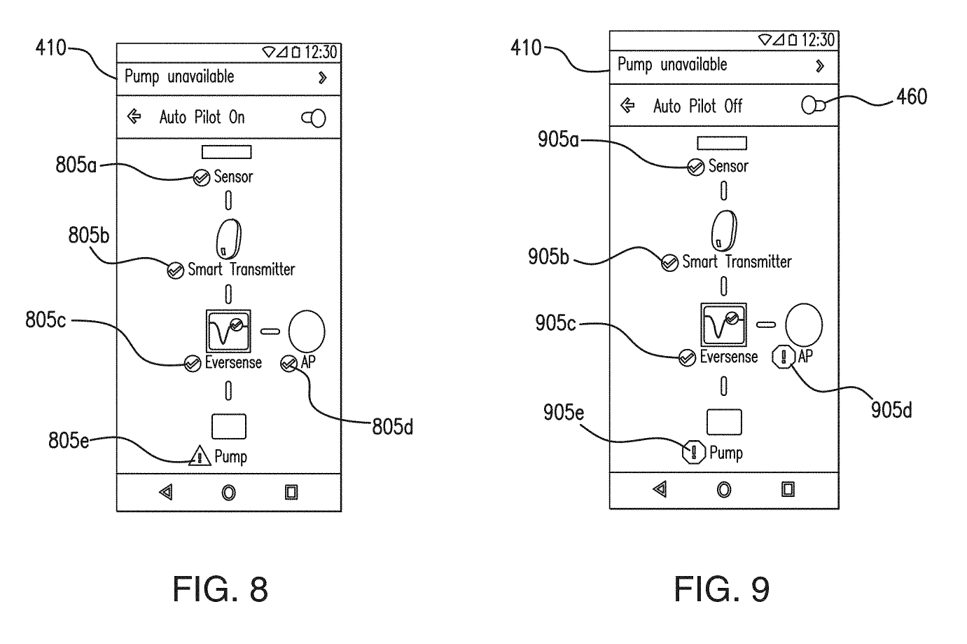

In some embodiments, a third additional state in the analyte monitoring and insulin delivery system 100 may comprise a pump buffer state, as shown in FIG. 8. In some embodiments, the pump buffer state may be caused by the insulin pump 105. Accordingly, FIG. 7A shows a status indication 805e of the insulin pump 105 showing a warning sign while the remaining status indications 805a-d show a normal sign. As shown in FIG. 8, the message bar 410 may display “Pump unavailable” according to the pump buffer state of the analyte monitoring and insulin delivery system 100.

In some embodiments, a fourth additional state in the analyte monitoring and insulin delivery system 100 may comprise a pump stopped state, as shown in FIG. 9. In the non-limiting example shown in FIG. 9, the AP switch 460 is turned off, which may cause the status indication 905d of the AP function to display a failure sign. In some embodiments, the pump stopped state may be caused by the insulin pump 105. Accordingly, FIG. 9 shows a status indication 905e of the insulin pump 105 showing a failure sign while the remaining status indications 905a-c show a normal sign. As shown in FIG. 9, the message bar 410 may display “Pump unavailable” according to the pump buffer state of the analyte monitoring and insulin delivery system 100.

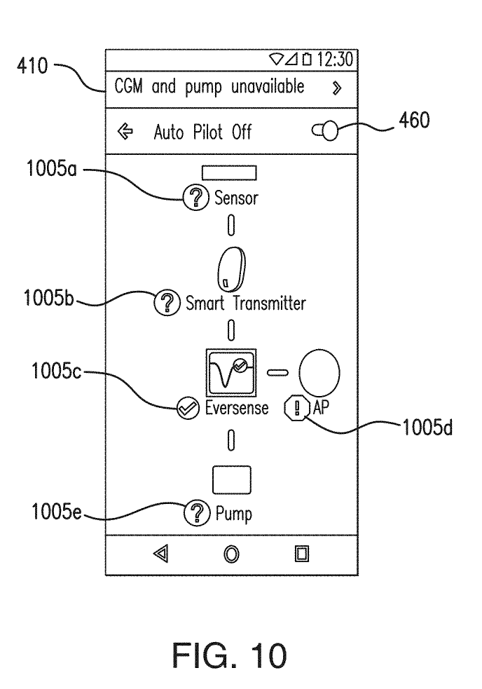

In some embodiments, a fifth additional state in the analyte monitoring and insulin delivery system 100 may comprise an analyte monitoring and pump unavailable state (e.g., CGM+Pump Unavailable state), as shown in FIG. 10. In the non-limiting example shown in FIG. 10, the AP switch 460 is turned off, which may cause the status indication 1005d of the AP function to display a failure sign. In some embodiments, the analyte monitoring and pump unavailable state may be caused by one or more of the analyte monitoring sensor 110, the transceiver 120, and the insulin pump 105. Accordingly, FIG. 10 shows a status indication 1005c of the MMA 305 showing a normal sign while the status indications 1005a, b, e for the analyte monitoring sensor 110, the transceiver 120, and the insulin pump 105 show a status unknown sign. As shown in FIG. 10, the message bar 410 may display “CGM and pump unavailable” according to the analyte monitoring and pump unavailable state of the analyte monitoring and insulin delivery system 100.

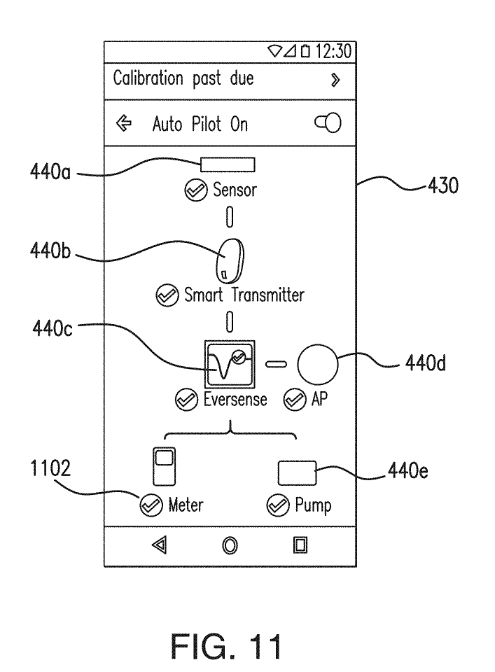

In some embodiments, the visual map 430, as shown in FIG. 4, may be adaptable and configured to remove and/or add icons corresponding to devices removed and/or added to the user's analyte monitoring and insulin delivery system 100. In a non-limiting embodiment, the analyte meter 107 may be added to the analyte monitoring and insulin delivery system 100, as shown above, for instance, in FIG. 1. Accordingly, a meter icon 1102 corresponding to the analyte meter 107 may be added to the visual map 430, as shown in FIG. 11 according to some embodiments.

Dashboard Screen

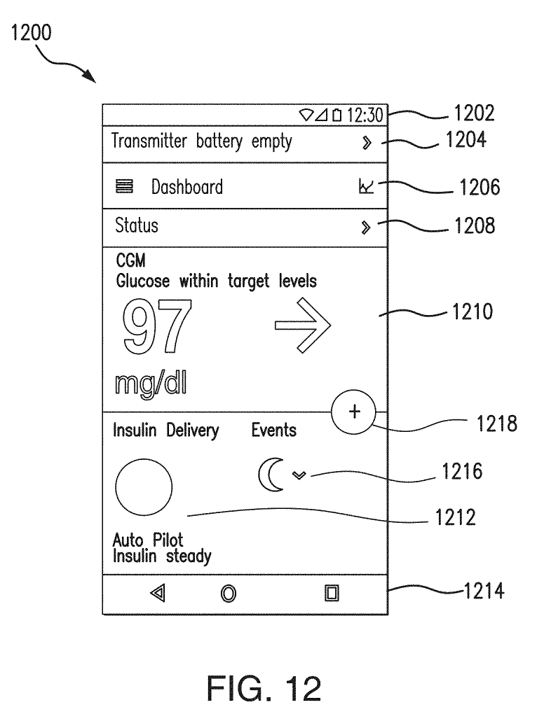

FIG. 12 shows an example dashboard screen embodying aspects of the present invention. FIG. 12 shows an example dashboard screen 1200 generated by the MMA 305 for display on the primary device 130 in accordance to an embodiment of the present invention. When a user gains access to his or her user account, the user may choose to view the dashboard screen 1200. Accordingly, the MMA 305 may configure the primary device 130 to present the dashboard screen 1200 on a display of the primary device 130. As shown in FIG. 12, the dashboard screen 1200 may display one or more of a device operation system status bar 1202, a message bar 1204, a title bar 1206, a status bar 1208, a CGM panel 1210, an insulin delivery panel 1212, an events panel 1216, an add event button 1218, and a device operating system navigation bar 1214, according to some embodiments.

In some embodiments, the device operation system status bar 1202 and the device operating system navigation bar 1214 may appear on one or more screens of the MMA 305. In some non-limiting embodiments, the device operation system status bar 1202 and the device operating system navigation bar 1214 may appear on every screen of the MMA 305. As a non-limiting example, the device operation system status bar 1202 and the device operating system navigation bar 1214 may appear on the dashboard screen 1200 and the system status screen 400 as described above, for instance, with reference to FIGS. 12 and 4A and 4B respectively. In some embodiments, the device operation system status bar 1202 comprises information indicating the current state of the primary device 130. In some embodiments, the device operating system navigation bar 1214 enables a user to navigate in between screens of the MMA 305 and/or navigate in between different mobile applications installed on the primary device 130. In some embodiments, the device operation system status bar 1202 and the device operating system navigation bar 1214 may be components available as part of a SDK for the operating system of the primary device 130.

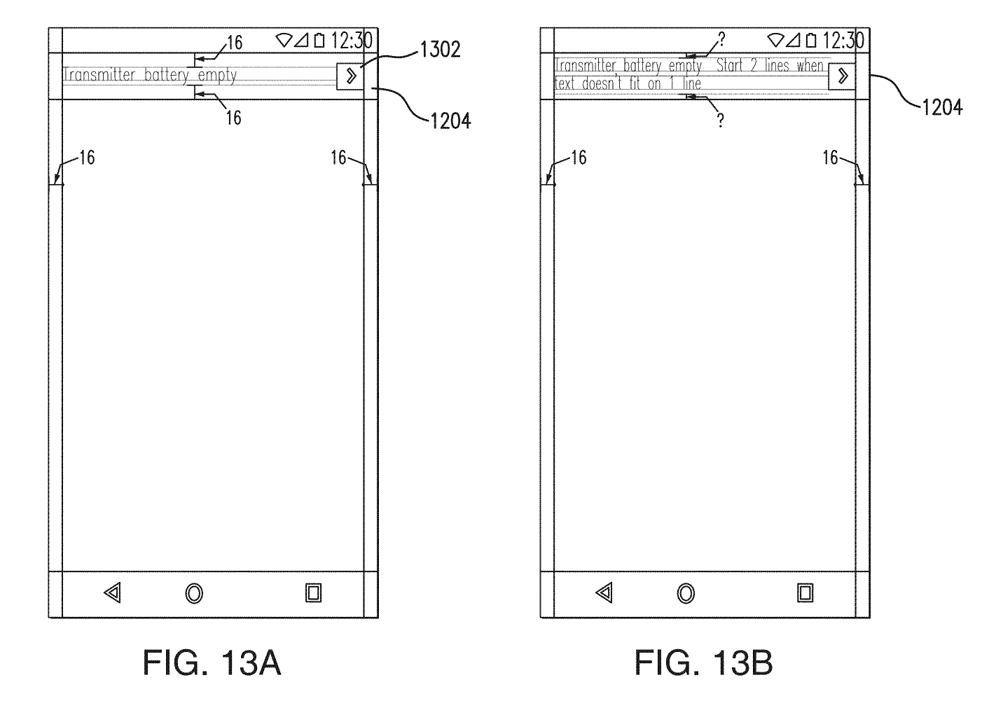

In some embodiments, the message bar 1204 may be configured to display system messages as described above with reference to FIG. 4A. In some embodiments, system messages may comprise messages regarding any information requiring the user's attention regarding the analyte monitoring and insulin delivery system 100. As a non-limiting example, FIG. 13A shows the message bar 1204 displaying “Transmitter battery empty.” Accordingly, the message bar 1204 may provide the user with a notification that the transceiver 120 of the analyte monitoring and insulin delivery system 100 requires a new battery and/or the transceiver 120 needs to be recharged. Other non-limiting examples of the message bar 1204 are described in the following description. In some embodiments, the message bar 1204 may comprise an arrow 1302 to indicate that a user may select (e.g., touch or tap) the message bar 1204. In some embodiments, the selectable region may not be restricted to the arrow 1302, and the entire message bar 1204 may be configured to detect a selection (e.g., contact) made by the user. In some embodiments, there may be more text than will fit on one line of the message bar 1204. In such embodiments, the first line of text may be moved up and stacked on a second line of text to form a message of two lines, as shown in FIG. 13B. In some embodiments, there may be no system message to be displayed in the message bar 1204. In such embodiments, the message bar 1204 containing no text remains displayed to the user.

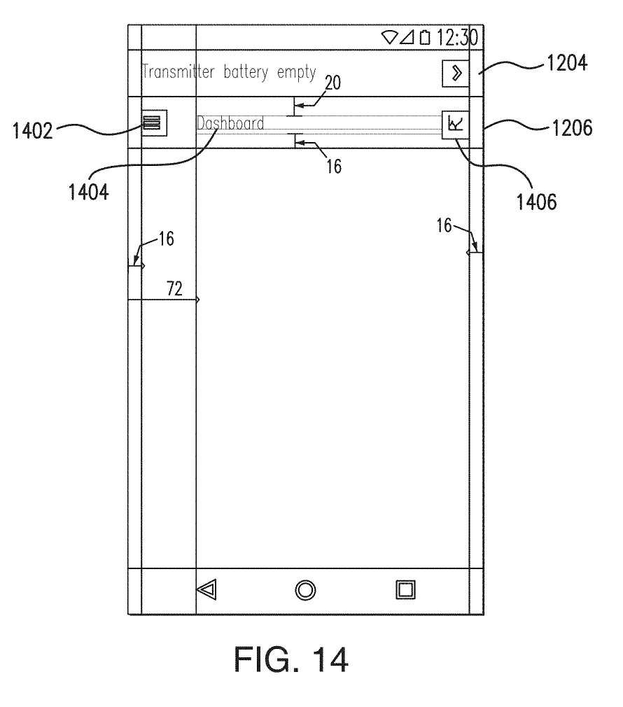

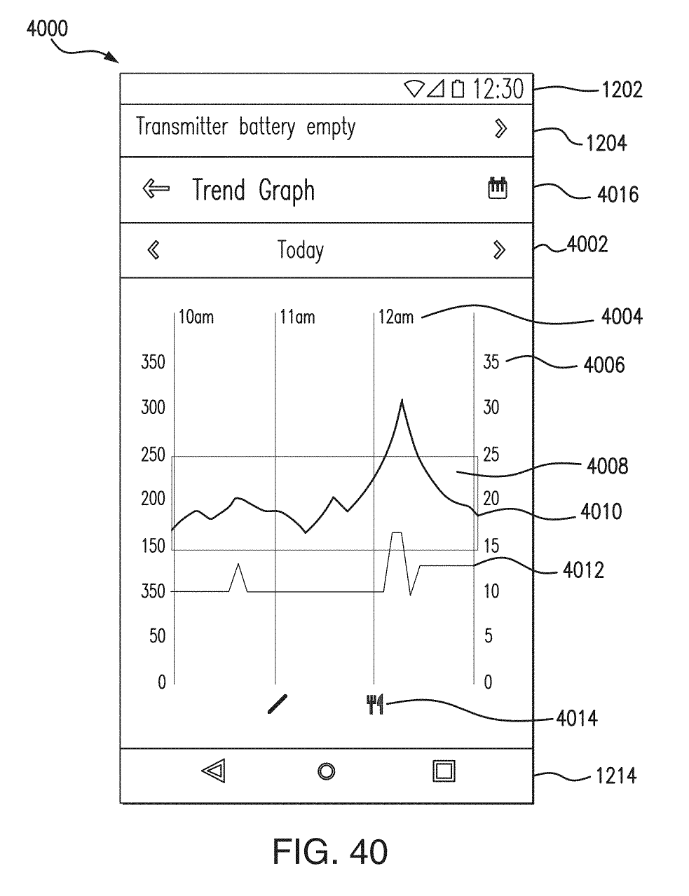

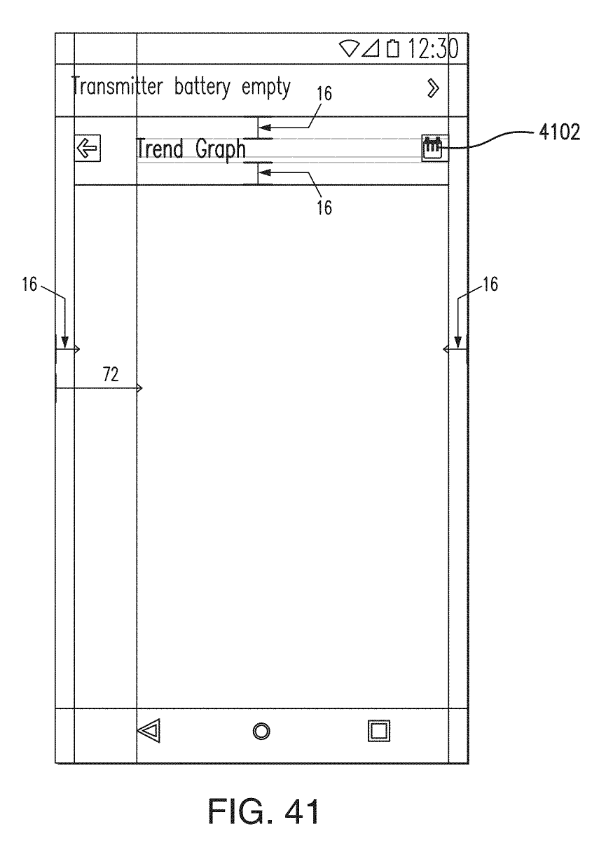

In some embodiments, the title bar 1206 may be displayed underneath the message bar 1204, as shown in FIG. 14. In some embodiments, the title bar 1206 may comprise a menu icon 1402, a title 1404 (e.g., “dashboard” to indicate that the user is viewing the dashboard screen), and a glucose trend graph icon 1406. In some embodiments, a user may select (e.g., touch or tap) the glucose trend graph icon 1406. In some non-limiting embodiments, upon detection of a selection of (e.g., contact made with) the glucose trend graph icon 1406, the MMA 305 may configure the primary display device 130 to present a trend graph screen 4000, as shown in FIG. 40.

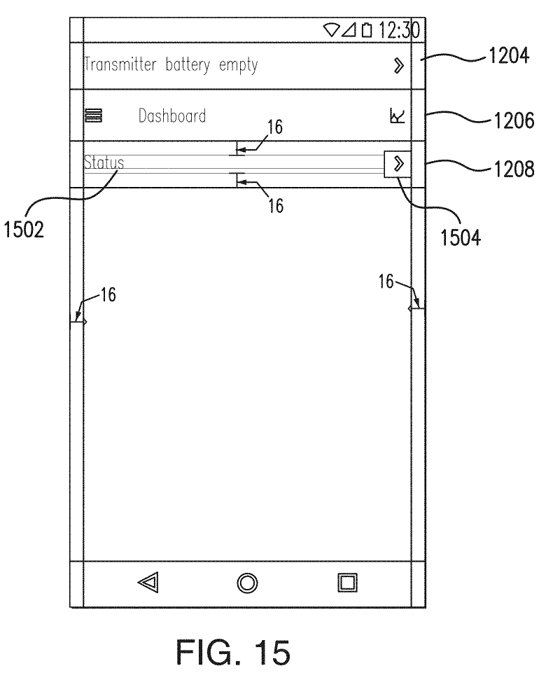

In some embodiments, the status bar 1208 may be displayed underneath the title bar 1206, as shown in FIG. 15. In some embodiments, the status bar 1208 may comprise a title 1502 and an arrow icon 1504. In some embodiments, the arrow 1504 may indicate that a user may select (e.g., touch) the status bar 1208. In some embodiments, the selectable region may not be restricted to the arrow 1504, and the entire status bar 1208 may be configured to detect a selection (e.g., contact) made by the user. In some non-limiting embodiments, upon detection of a selection (e.g., contact) made with the status bar 1208, the MMA 305 may configure the primary device 130 to present the system status screen 400 on a display of the primary device 130, as described above with reference to FIGS. 4A and 4B.

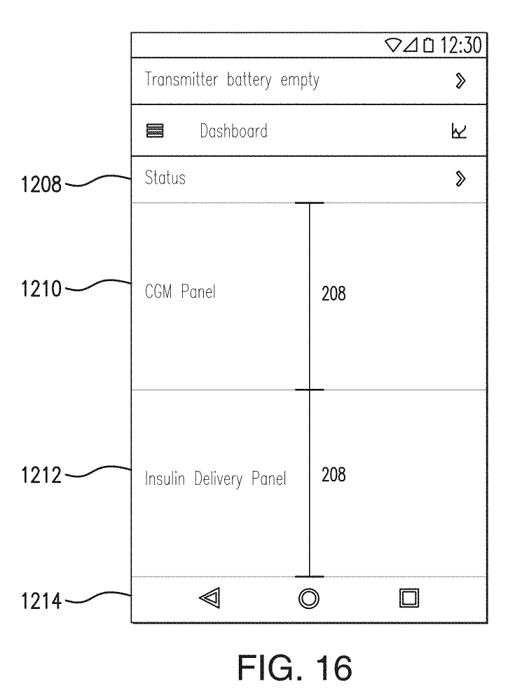

In some embodiments, the remaining space beneath the status bar 1208 and above the device operation system navigation bar 1214 may be divided into two equal portions, as shown in FIG. 16. The CGM Panel 1210 may be displayed in the top portion and the insulin delivery panel 1212 may be displayed in the bottom portion. In some embodiments, the insulin delivery panel 1212 and the events panel 1216 may be displayed in the bottom portion. However, it is not required that the top portion and the bottom portion be equal, and the area of the top portion and the bottom portion may vary for alternative embodiments. In some embodiments, the bottom portion may be divided horizontally into a left column and a right column. In some embodiments, the left column and the right column may be of equal width. In some embodiments, the insulin delivery panel 1212 may be displayed in the left column of the bottom portion and the events panel may be displayed in the right column of the bottom portion.

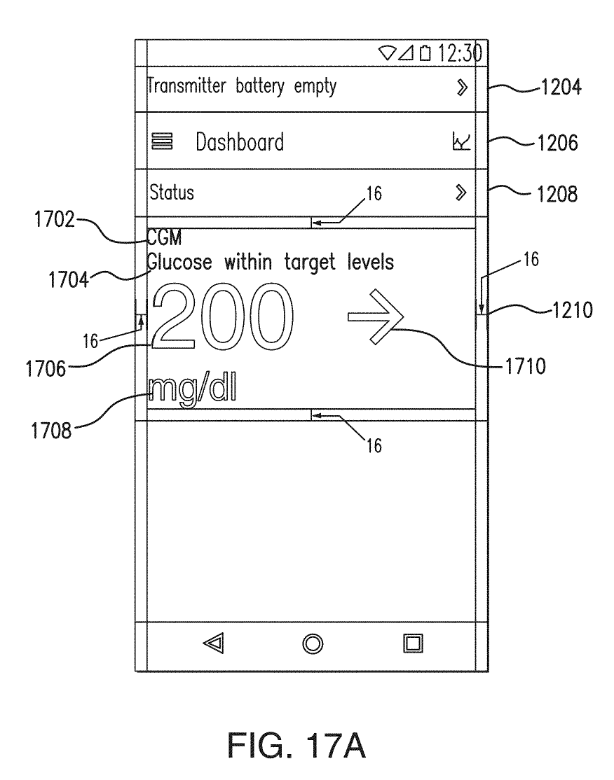

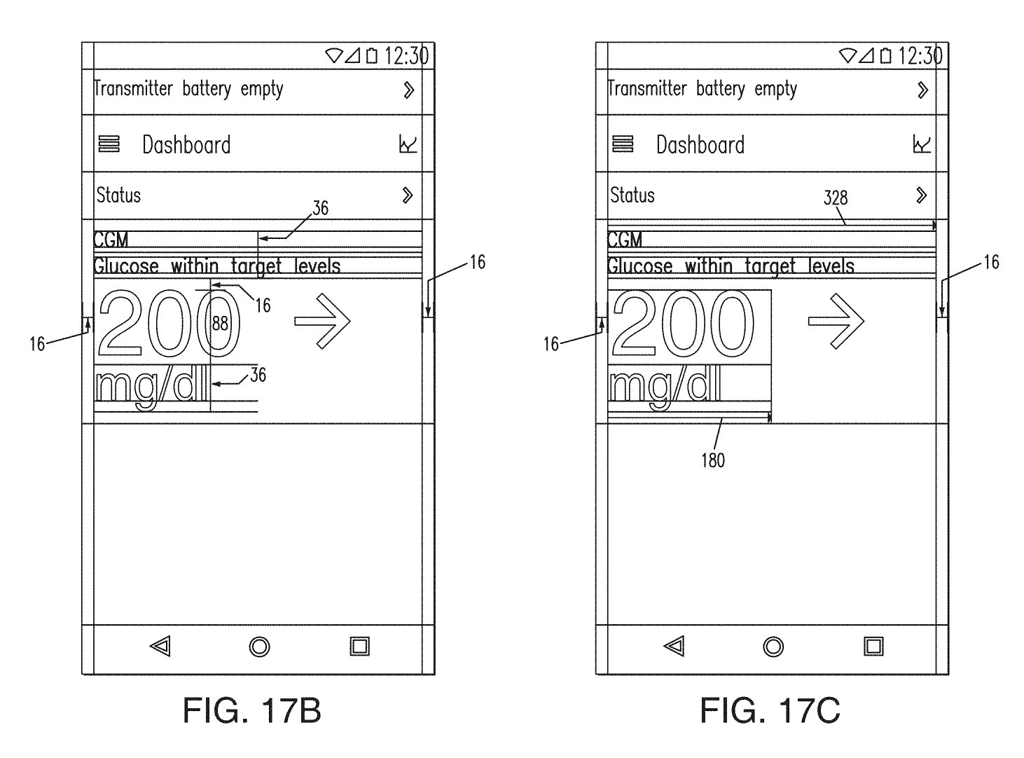

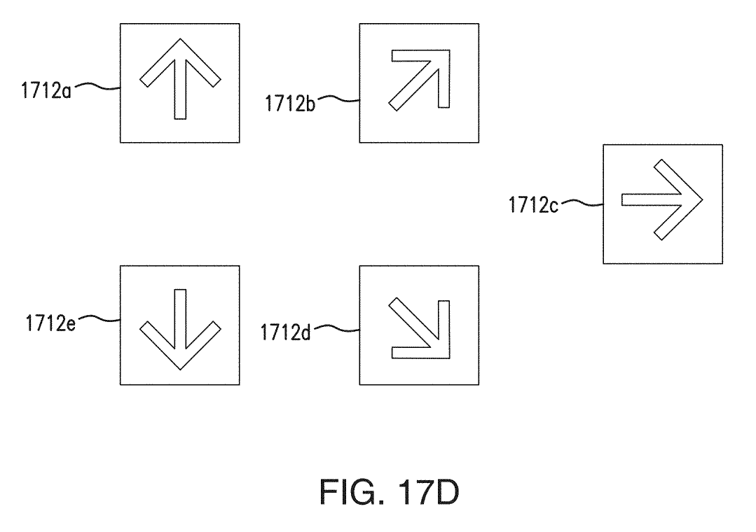

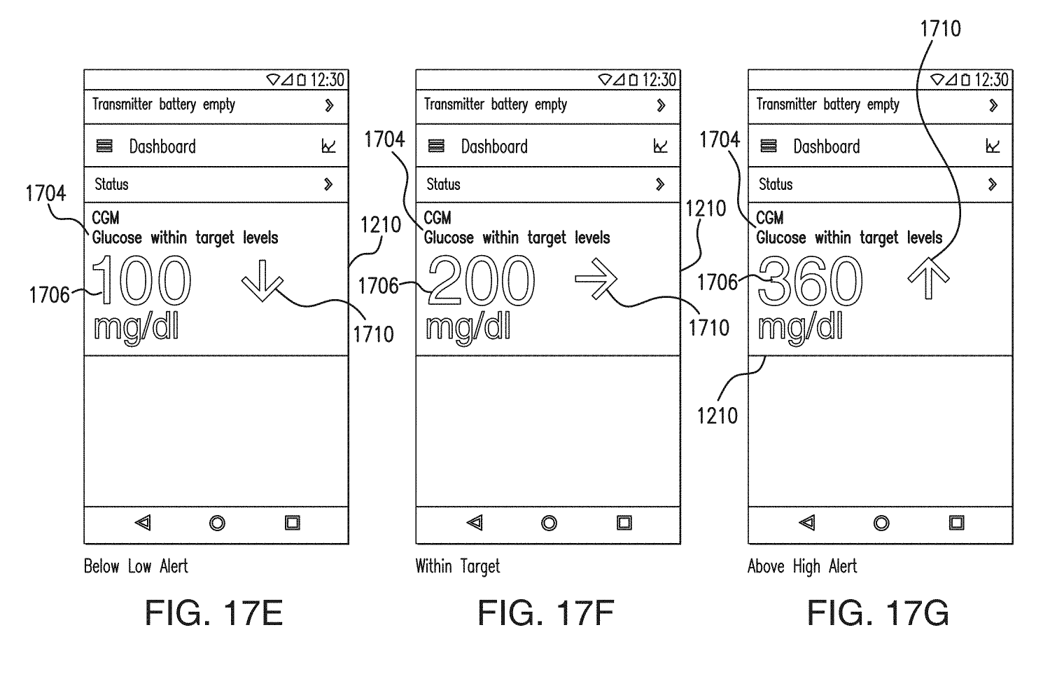

In some embodiments, the CGM panel 1210 may be displayed underneath the status bar 1208, as shown in FIG. 17A. In some embodiments, the CGM panel 1210 may be configured to display a title 1702 with a description 1704 beneath. In some embodiments, the CGM panel 1210 may be configured to display a glucose reading number (“CGM number”) 1706, a unit label 1708 beneath it, and a trend arrow 1710. In some embodiments, the CGM number 1706 may be configured to accommodate three digits, with or without a decimal place. In some embodiments, the CGM number may display a European standard comma in place of the United States standard period. In some embodiments, the unit label 1708 may display one or more of mg/dl and mmol/L. In a non-limiting example, the vertical dimensions and the horizontal dimensions of the CGM panel 1210 are shown in FIGS. 17B-C, respectively.

FIG. 17D shows an example of the trend arrow 1710. In some embodiments, the trend arrow 1710 may be depicted in five different configurations 1712a-e that signify direction (up, down, neutral) and rate (rapidly, very rapidly slow, slow, very slow, and stable) of analyte change according to an embodiment of the present invention. In some embodiments, there may be times when the trend arrow 1710 may not be displayed due to, for example, there being insufficient sensor values available for the trend calculation. In some embodiments, the trend arrow 1710 displayed in a horizontal orientation 1712c (approximately 0° along the horizontal direction of the GUI display) may indicate that the glucose level is changing gradually (e.g., trend steady), such as, for example, at a rate between −1.0 mg/dL and 1.0 mg/dL per minute. In some embodiments, the trend arrow 1710 displayed slightly in the upwards direction 1712b (approximately 45° up from the horizontal direction of the GUI display) may indicate that the glucose level is rising moderately (e.g., trend up slow), such as, for example, at a rate between 1.0 mg/dL and 2.0 mg/dL per minute. In some embodiments, the trend arrow 1710 displayed slightly in the downwards direction 1712d (approximately 45° down from the horizontal direction of the GUI display) may indicate that the glucose level is falling moderately (e.g., trend down slow), such as, for example, at a rate between 1.0 mg/dL and 2.0 mg/dL per minute. In some embodiments, the trend arrow 1710 displayed in a vertical direction 1712a (approximately 90° up from the horizontal direction of the GUI display) may indicate that the glucose level is rising very rapidly (e.g., trend up fast), such as, for example, at a rate more than 2.0 mg/dL per minute. In some embodiments, the trend arrow 1710 displayed in a downwards direction 1712e (approximately 90° down from the horizontal direction of the GUI display) may indicate that the glucose level is falling very rapidly (e.g., trend down fast), such as, for example, at a rate more than 2.0 mg/dL per minute.

In some embodiments, a background of the CGM panel 1210 may comprise three different colors depending on the state of the CGM number 1706. In some embodiments, the CGM number 1706 may indicate a low CGM measurement, which may necessitate an alert. In a non-limiting example, the CGM panel 1210 may display 100 mg/dl, which may be a glucose level below a low alert level, as shown in FIG. 17E. In such embodiments, the description 1704 may display that “glucose below level alert” and the background color of the CGM panel 1210 may turn red. Accordingly, the trend arrow 1710 is displayed in the downwards direction 1712e. In some embodiments, the CGM number 1706 may indicate that a CGM measurement which is within target levels. In a non-limiting example, the CGM panel 1210 may display 200 mg/dl, which may be a glucose level within target levels. In such embodiments, the description 1704 may display “glucose within target levels” and the background color of the CGM panel 1210 may turn green. Accordingly, the trend arrow 1710 is displayed in a horizontal orientation 1712c. In some embodiments, the CGM number 1706 may indicate a high CGM measurement which may necessitate an alert. In a non-limiting example, the CGM panel 1210 may display 360 mg/dl, which may be a glucose level above a high alert level. In such embodiments, the description 1704 may display that “glucose above high alert level” and the background color of the CGM panel 1210 may turn amber. Accordingly, the trend arrow 1710 may be displayed in the vertical direction 1712a.

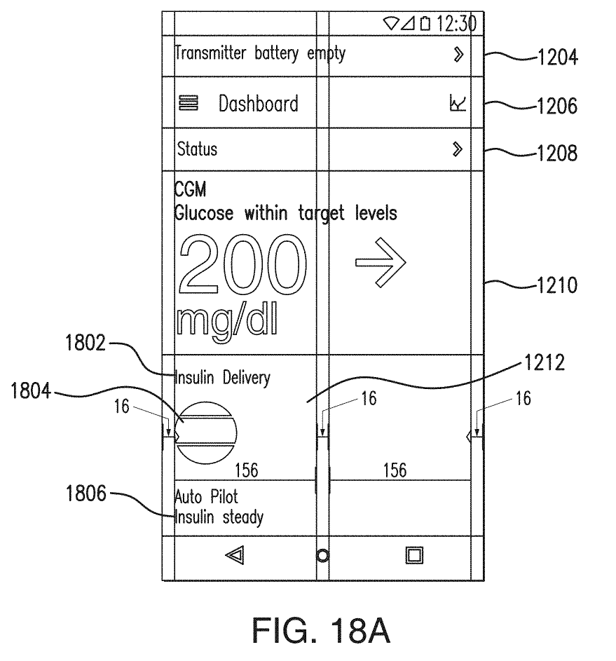

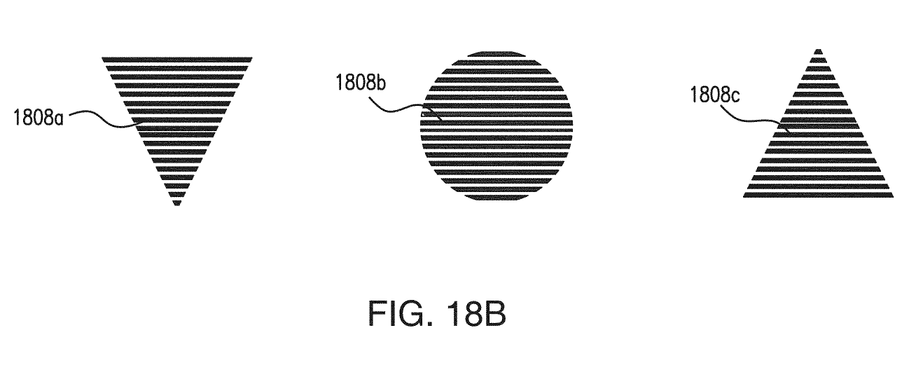

In some embodiments, the insulin delivery panel 1212 may be displayed underneath the CGM panel 1210, as shown in FIG. 18A. In some embodiments, the insulin delivery panel 1212 may comprise a title 1802, an Auto Pilot (AP) icon 1804, and a description 1806. In some non-limiting embodiments, the title may display “insulin delivery,” as shown in FIG. 18. In some embodiments, the description 1806 provides a description of the current status the AP icon 1804, where the AP icon 1804 displays an indication of the current status of the AP function, as described above, for instance in FIG. 4A and related descriptions.

FIG. 18B shows an example of the AP icon 1804. In some embodiments, the AP icon 1804 may be depicted in three different configurations 1808a-c that signify direction (up, down, neutral) and rate (increase, decrease, and stable) of the amount of insulin calculated by the AP function and delivered by the insulin pump 105, according to an embodiment of the present invention. In some embodiments, the AP icon 1804 displayed as a downward pointing triangle 1808a may indicate that the rate of insulin calculated by the AP function and delivered by the insulin pump 105 has decreased. Accordingly, the description 1806 may provide a description to the user that the insulin rate has decreased. In some embodiments, the AP icon 1804 displayed as a circle 1808b may indicate that the rate of insulin calculated by the AP function and delivered by the insulin pump 105 is steady. Accordingly, the description 1806 may provide a description to the user that the insulin rate is steady, as shown in FIG. 18A. In some embodiments, the AP icon 1804 displayed as an upward pointing triangle 1808c may indicate that the rate of insulin calculated by the AP function and delivered by the insulin pump 105 has increased. Accordingly, the description 1806 may provide a description to the user that the insulin rate has increased.

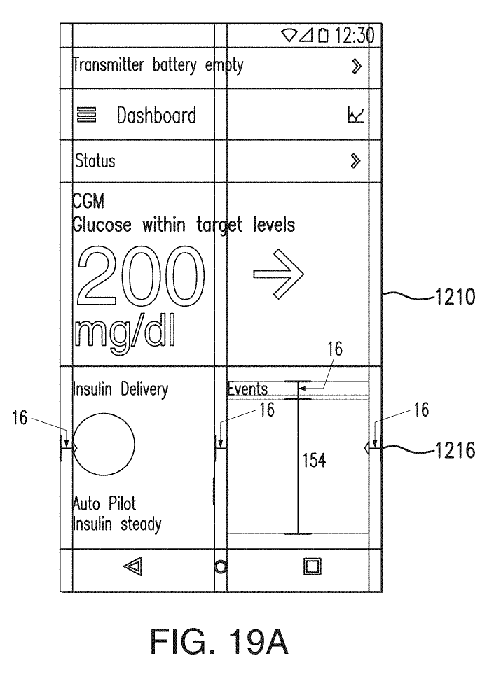

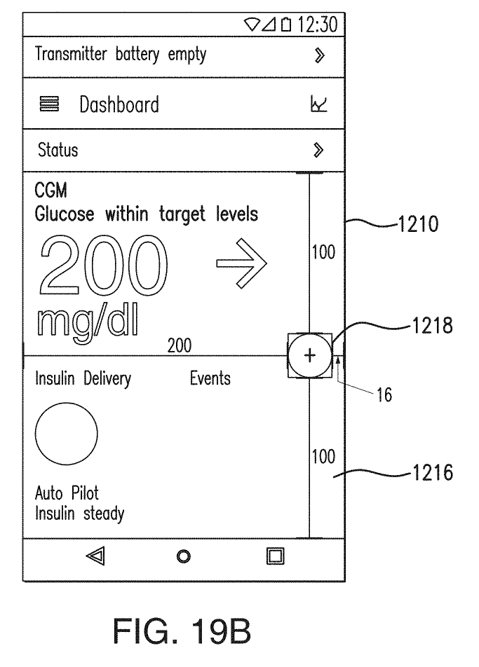

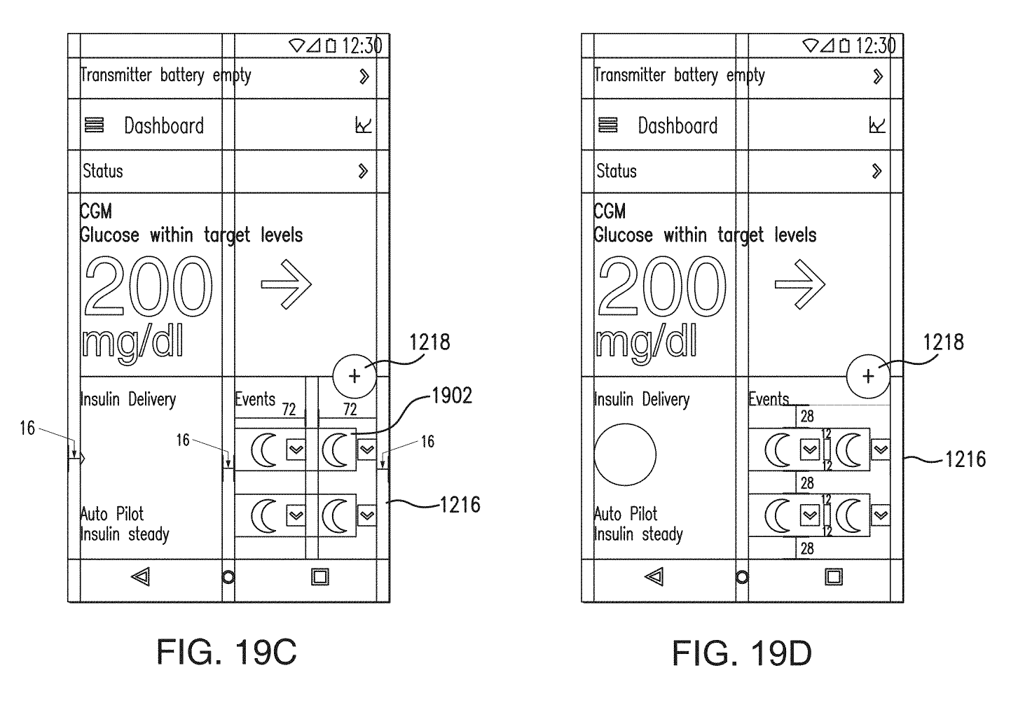

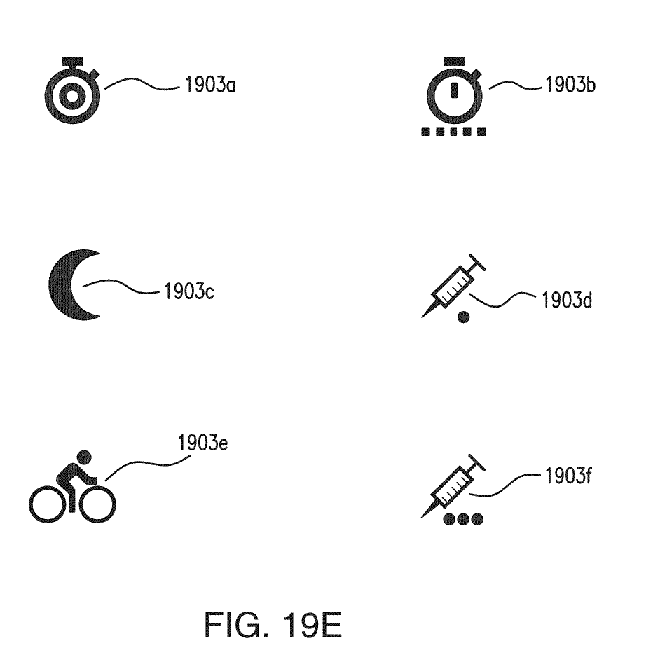

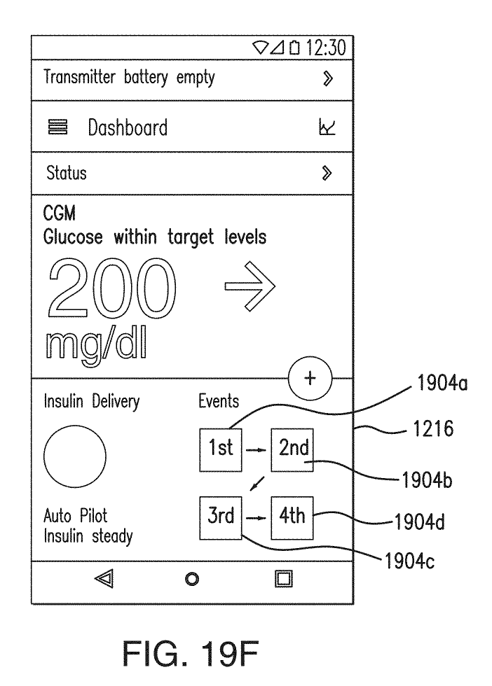

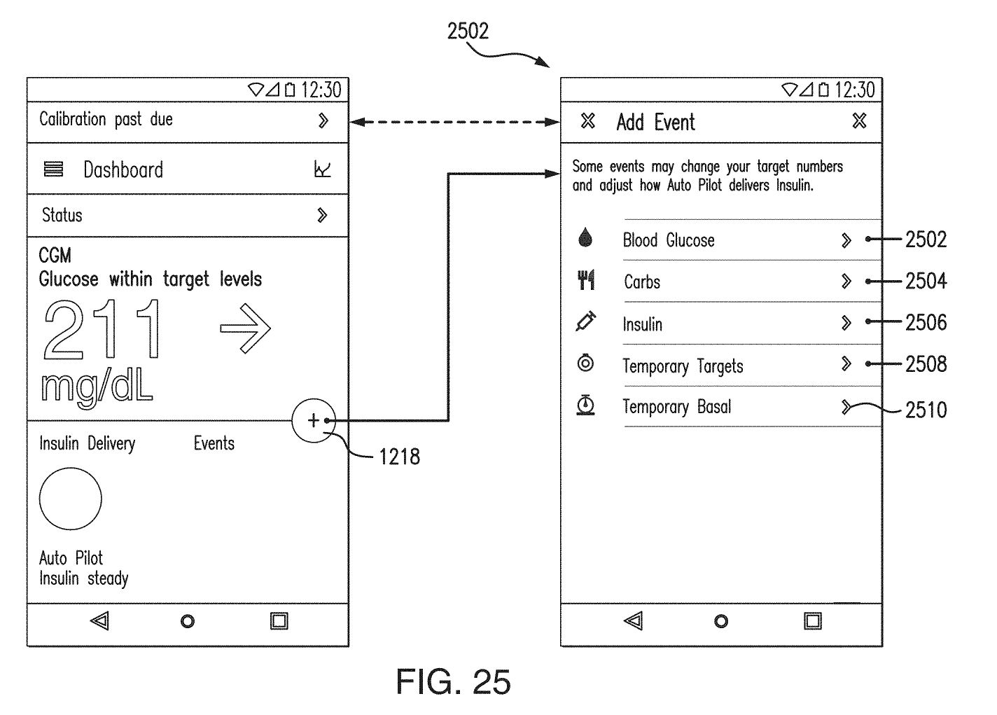

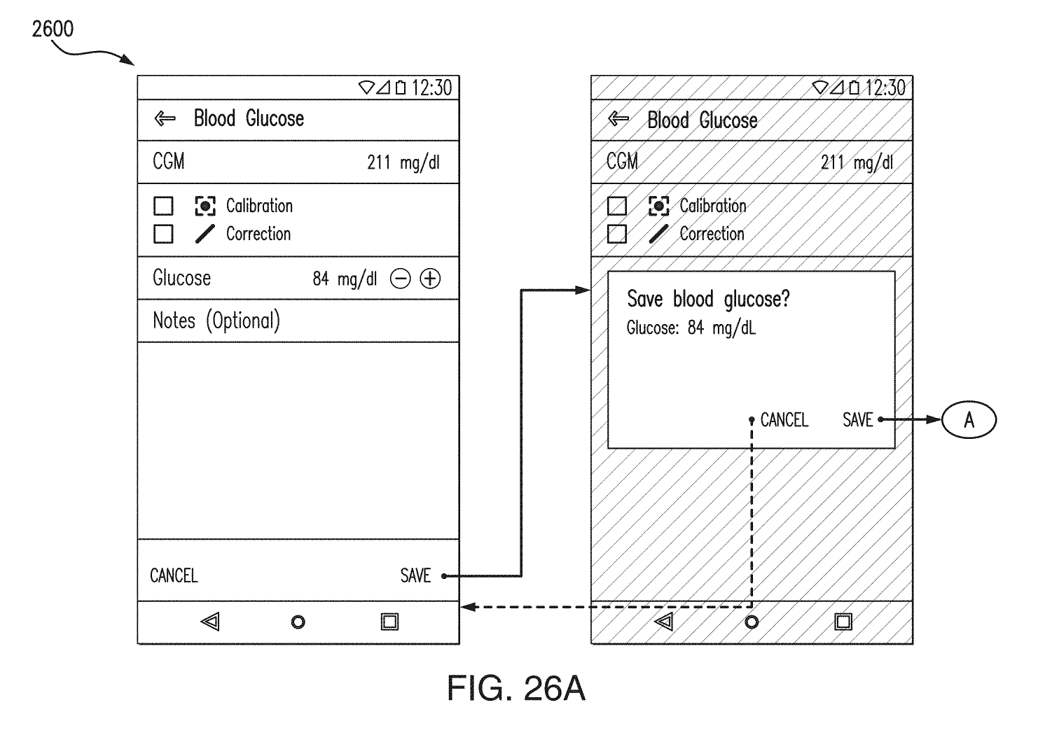

In some embodiments, the events panel 1216 may be displayed underneath the CGM panel 1210, as shown in FIG. 19A. In some embodiments, the add event button 1218, as shown in FIG. 19B, may provide a main action for users in the events panel 1216. In some embodiments, the add event button 1218 may be placed vertically in between the CGM panel 1210 and the events panel 1216. In some embodiments, the add event button 1218 may comprise a floating action which may be a component available as part of the SDK for the operating system of the primary device 130. In some embodiments, a user may select (e.g., touch) the add event button 1218. In some non-limiting embodiments, upon detection of a selection of (e.g., contact made with) the add event button 1218, the MMA 305 may configure the primary device 130 to present an add event screen 2500, as shown in FIG. 25. In some embodiments, the events may comprise at least one or more of duration events and point-in-time events. Any event added by using the add event screen 2500, as will be described in further detail in FIGS. 25-39, may be represented as an event icon 1902 and displayed in the events panel 1216, as shown in FIGS. 19C-D. In some embodiments, only duration events may appear as event icons on the event panel 1216. In some embodiments, point-in-time event (e.g., glucose, correction, meal, hypo, and insulin) may not appear as event icons on the event panel 1216. Some non-limiting exemplary event icons are shown in FIG. 19E. For example and without limitation, the event icons may include one or more of a target generic event icon 1903a, a basal event icon 1903b, a sleep target event icon 1903c, a standard bolus event icon 1903d, an exercise target event icon 1903e, and an extended bolus event icon 1903f In some embodiments, events icons may appear in the order the events are added. As shown in FIG. 19F, a first event icon 1904a corresponding to a first added event may appear in a upper left quadrant of the events panel 1216, a second event icon 1904b corresponding to a second added event may appear in a upper right quadrant of the events panel 1216, a third event icon 1904c corresponding to a third added event may appear in a lower left quadrant of the events panel 1216, and a fourth event icon 1904d corresponding to a fourth added event may appear in a lower right quadrant of the events panel 1216, according to some embodiments.

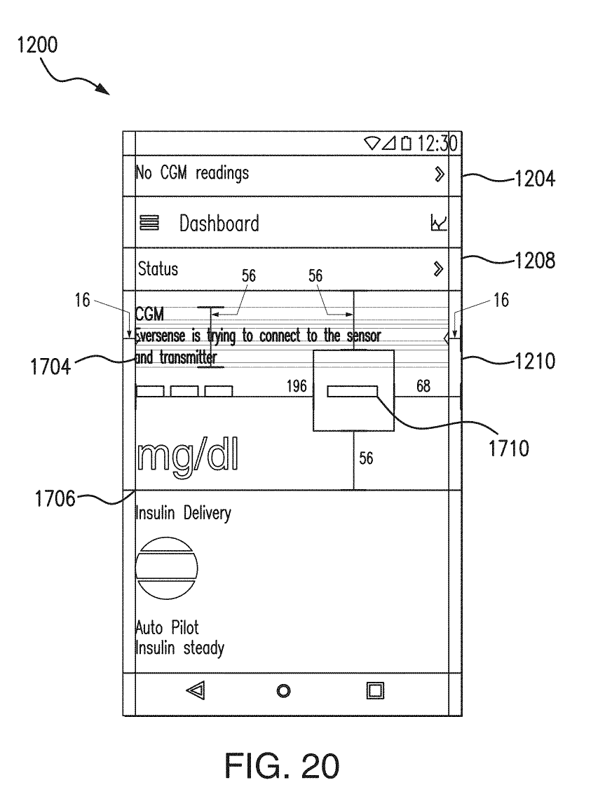

In some embodiments, the analyte monitoring and insulin delivery system 100 may have five additional states, as described above, for instance, in FIGS. 6-10. The first additional state in the analyte monitoring and insulin delivery system 100 may comprise the analyte monitoring buffer state (e.g., CGM buffer), according to some embodiments. In such embodiments, the dashboard screen 1200 may be configured as shown in FIG. 20. In some embodiments, the message bar 1204 may be configured to display an indication that CGM readings are not available and the description 1704 of the CGM Panel 1210 may be configured to indicate that the MMA 305 is trying to connect to the analyte monitoring sensor 110 and/or the transceiver 120. In some embodiments, the CGM number 1706 and the trend arrow 1710 may be displayed in flat horizontal bars to indicate the analyte monitoring buffer state. In some non-limiting embodiments, selecting (e.g., touching) the status bar 1208 may trigger the MMA 305 to present the system status screen 400 on the display of the primary device 130, as described above with reference to FIGS. 6A and 6B.

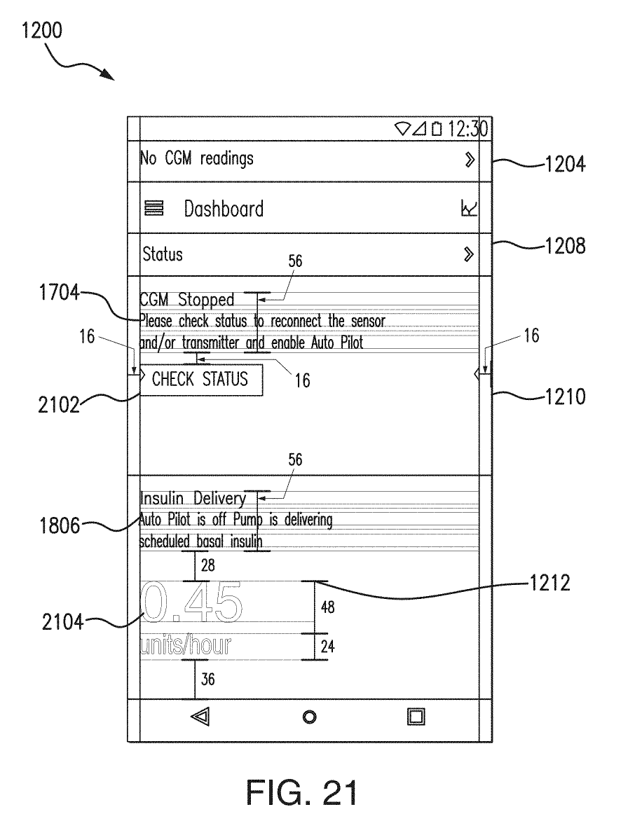

In some embodiments, the second additional state in the analyte monitoring and insulin delivery system 100 may comprise the analyte monitoring stopped state (e.g., CGM stopped). In such embodiments, the dashboard screen 1200 may be configured as shown in FIG. 21. In some embodiments, the message bar 1204 may be configured to display an indication that CGM readings are not available. In some embodiments, the description 1704 of the CGM Panel 1210 may be configured to indicate that the analyte monitoring sensor 110 and/or the transceiver 120 may need to be reconnected. In some embodiments, the description 1704 of the CGM Panel 1210 may be configured to further indicate that the AP function should be enabled. In some embodiments, the CGM number 1706 and the trend arrow 1710 may be replaced with a check status button 2102. In some embodiments, the insulin delivery panel 1212 may indicate that the AP function is off. In such embodiments, the insulin pump 105 may deliver scheduled basal insulin based on a predetermined configuration. Accordingly, the description 1806 of the insulin delivery panel 1212 may be configured to indicate that the insulin pump 105 is delivering scheduled basal insulin. Further, the AP icon 1804 may be replaced with a delivery rate 2104 indicating the schedule basal insulin delivery rate. As a non-limiting example, the delivery rate 2104 may be display 0.45 units/hour. In some non-limiting embodiments, selecting (e.g., touching) the status bar 1208 may trigger the MMA 305 to present the system status screen 400 on the display of the primary device 130, as described above with reference to FIGS. 7A and 7B.

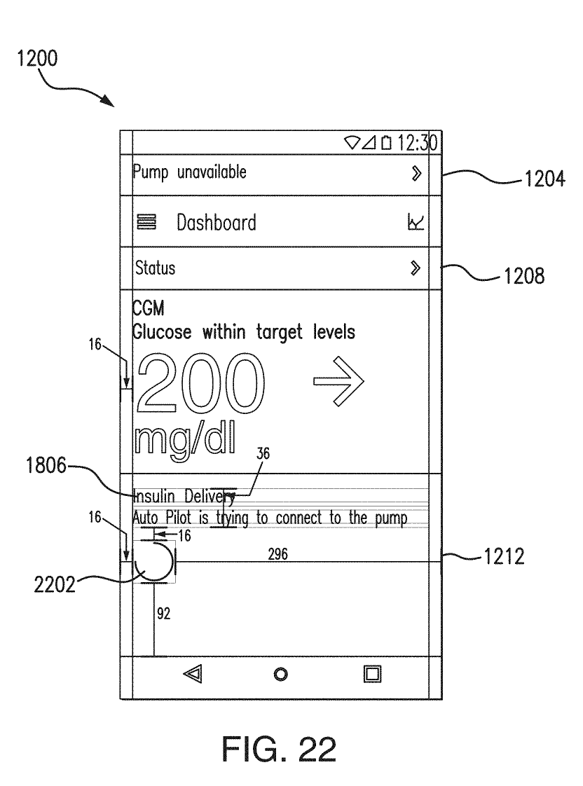

In some embodiments, the third additional state in the analyte monitoring and insulin delivery system 100 may comprise the pump buffer state. In such embodiments, the dashboard screen 1200 may be configured as shown in FIG. 22. In some embodiments, the message bar 1204 may be configured to display an indication that the insulin pump 105 is not available. In some embodiments, the insulin delivery panel 1212 may indicate that the AP function is trying to connect the insulin pump 105. In such embodiments, the description 1806 of the insulin delivery panel 1212 may be configured to indicate that the AP function is connecting to the insulin pump 105. Accordingly, the AP icon 1804 may be replaced with a circular progress indicator 2202 indicating that the AP function is connecting to the insulin pump 105. In some embodiments, selecting (e.g., touching) the status bar 1208 may trigger the MMA 305 to present the system status screen 400 on the display of the primary device 130, as described above with reference to FIG. 8.

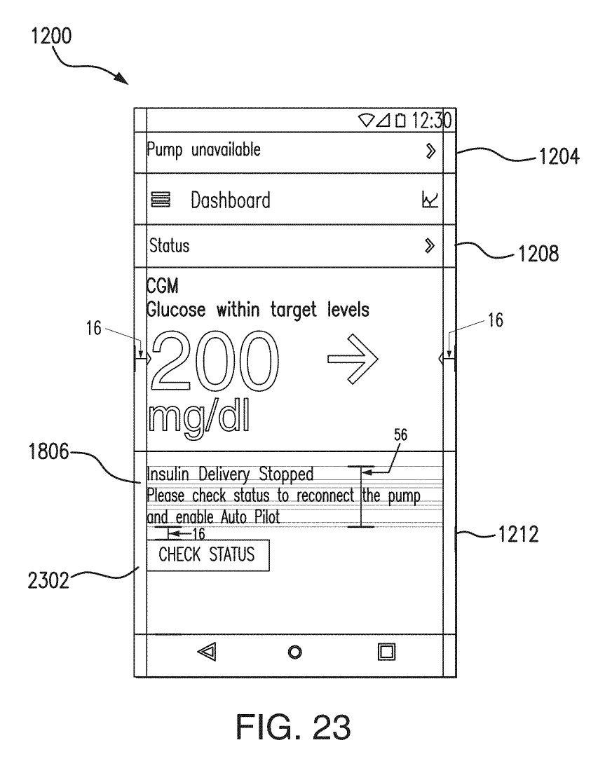

In some embodiments, the fourth additional state in the analyte monitoring and insulin delivery system 100 may comprise the pump stopped state. In such embodiments, the dashboard screen 1200 may be configured as shown in FIG. 23. In some embodiments, the message bar 1204 may be configured to display an indication that the insulin pump 105 is unavailable. In some embodiments, the description 1806 of the insulin delivery panel 1212 may indicate that insulin delivery has stopped and the insulin pump 105 needs to be reconnected. In some embodiments, the description 1806 of the insulin delivery panel 1212 may be configured to further indicate that the AP function should be enabled. Accordingly, the AP icon 1804 may be replaced with a check status button 2302. In some non-limiting embodiments, selecting the status bar 1208 may trigger the MMA 305 to present the system status screen 400 on the display of the primary device 130, as described above with reference to FIG. 9.

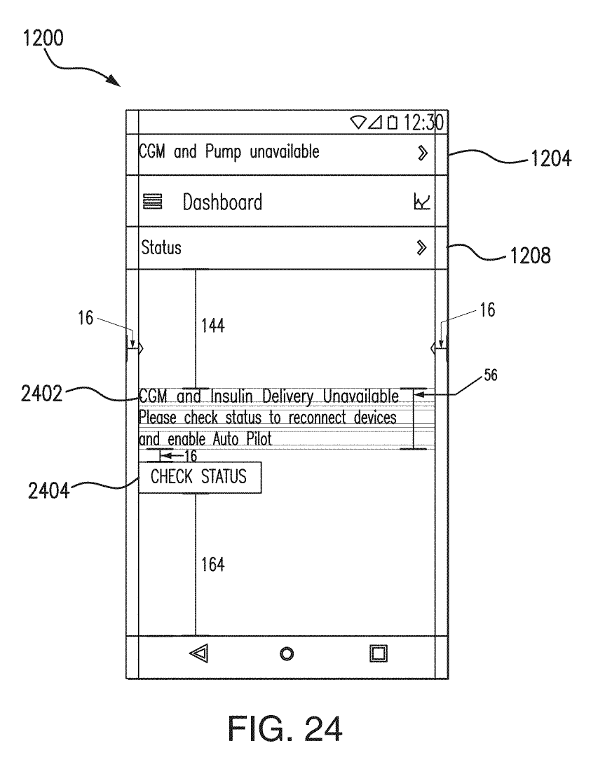

In some embodiments, the fifth additional state in the analyte monitoring and insulin delivery system 100 may comprise the analyte monitoring and pump unavailable state (e.g., CGM+Pump Unavailable state). In such embodiments, the dashboard screen 1200 may be configured as shown in FIG. 24. In some embodiments, the message bar 1204 may be configured to display an indication that the analyte monitoring and the insulin pump 105 are unavailable. In some embodiments, the insulin delivery panel 1212 and the CGM panel 1210 may be replaced with a message 2402 and a check status button 2404. In a non-limiting example, the message 2402 may be configured to indicate that the analyte monitoring and the insulin pump 105 are unavailable and that one or more of the analyte monitoring sensor 110, the transceiver 120, and the insulin pump 105 needs to be reconnected. In some embodiments, the message 2402 may be configured to further indicate that the AP function should be enabled. In some embodiments, selecting the status bar 1208 may trigger the MMA 305 to present the system status screen 400 on the display of the primary device 130, as described above with reference to FIG. 10.

FIG. 25 shows the add events screen 2500 according to an embodiment of the present invention. In some embodiments, the add events screen 2500 may comprise at least one or more of a blood glucose event button 2502, a carbohydrates event button 2504, an insulin event button 2506, a temporary targets button 2508, and a temporary basal button 2510. In some embodiments, one or more of the buttons may be selected by a user (e.g., by touching or tapping a button).

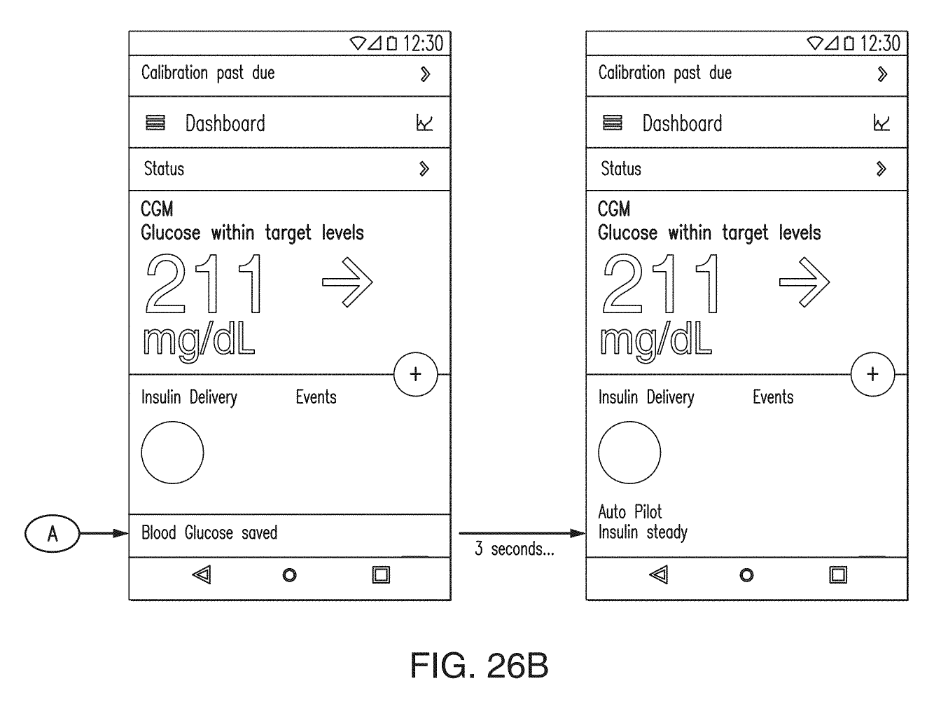

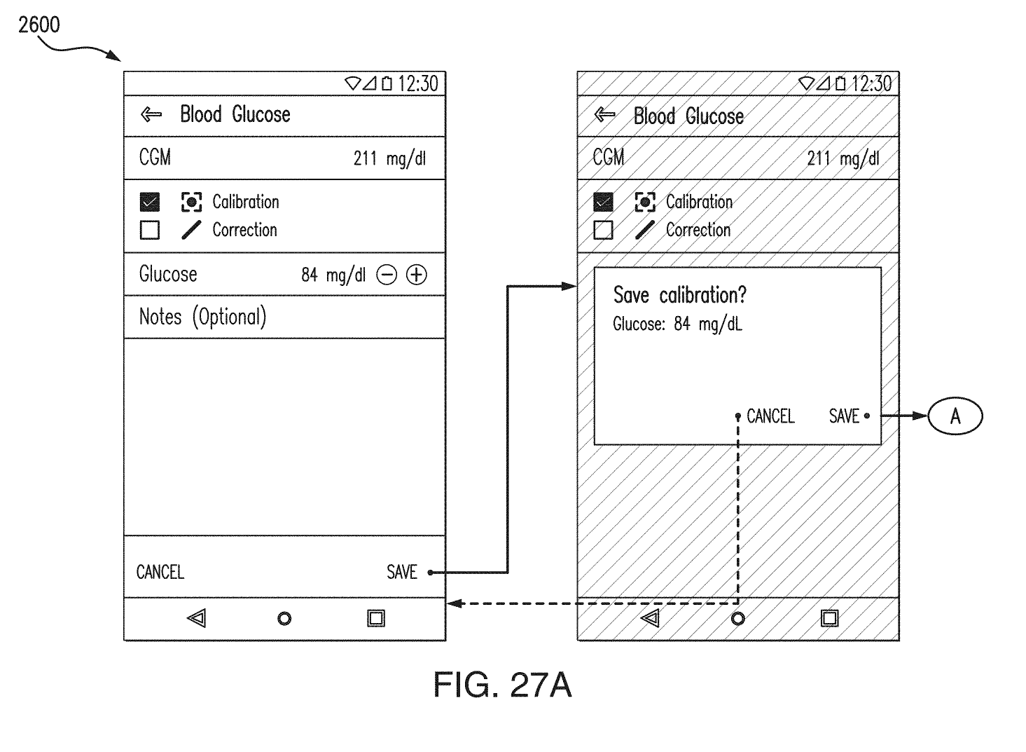

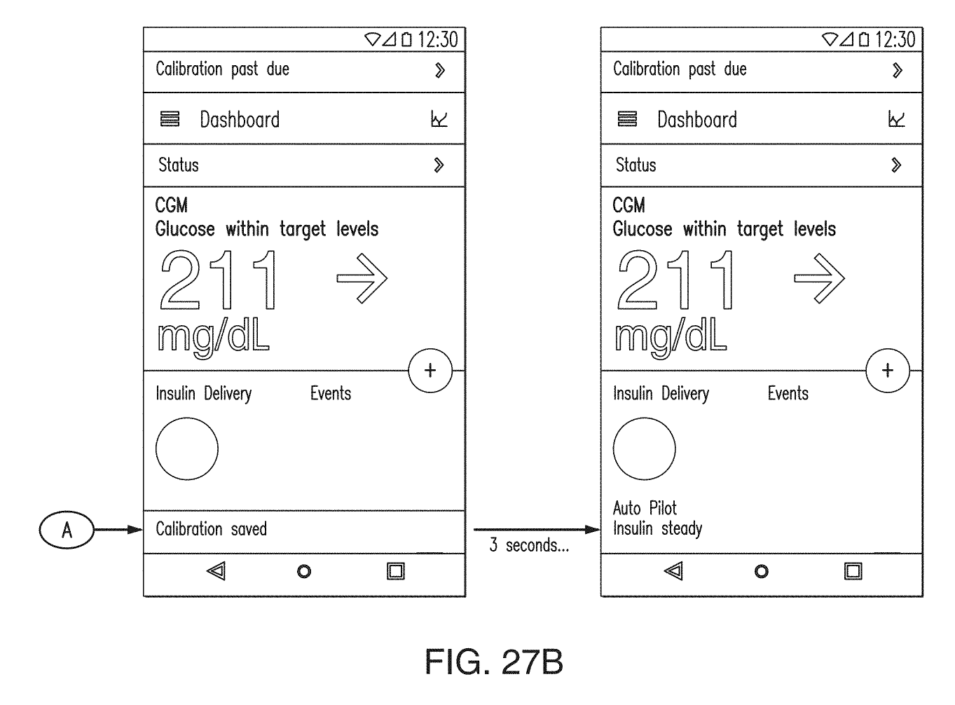

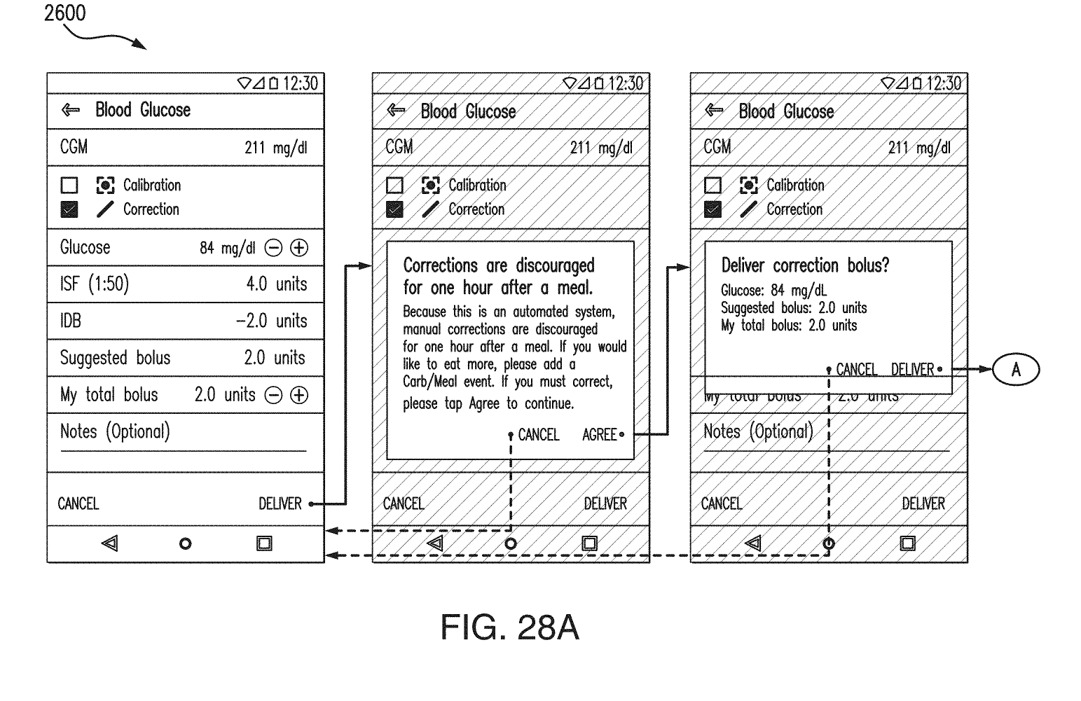

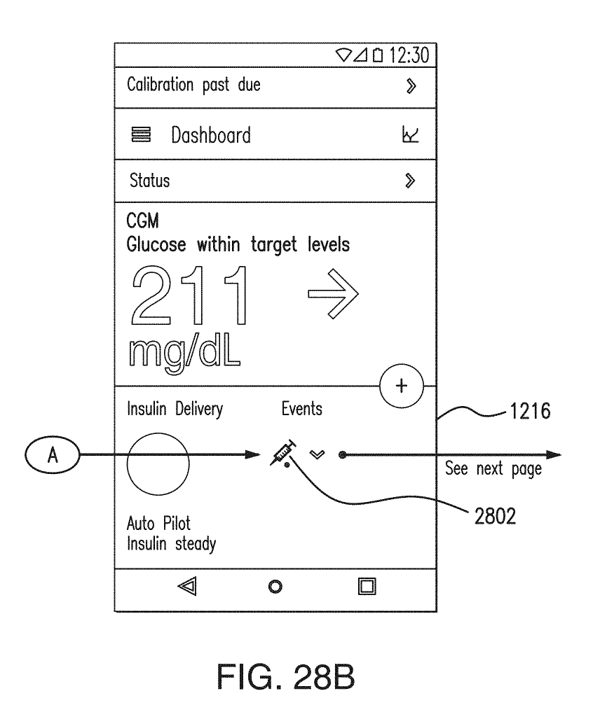

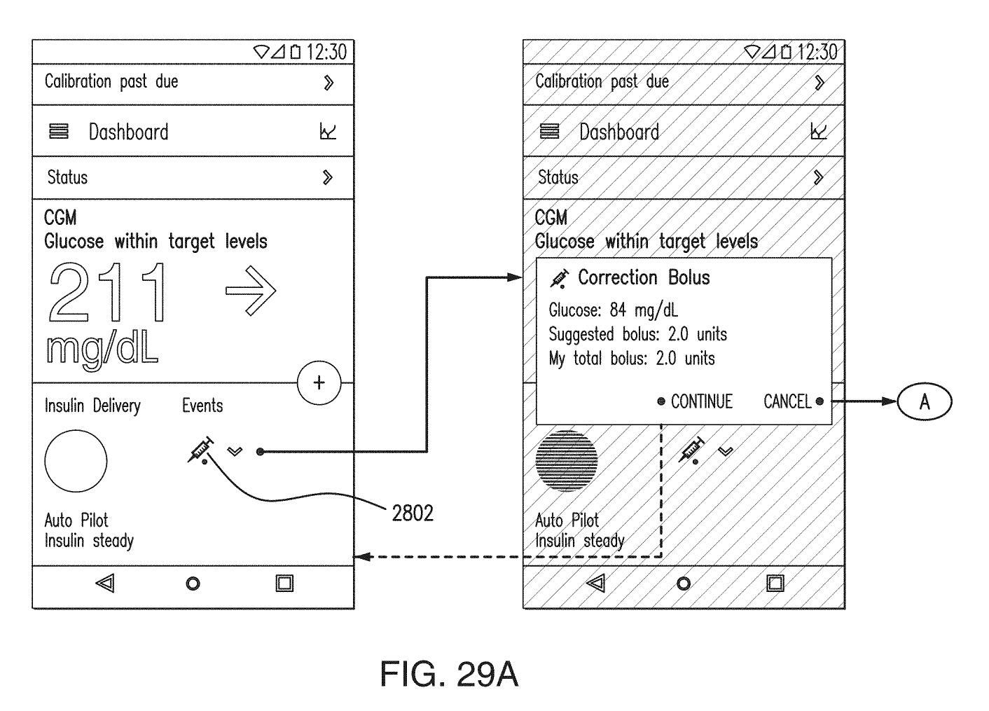

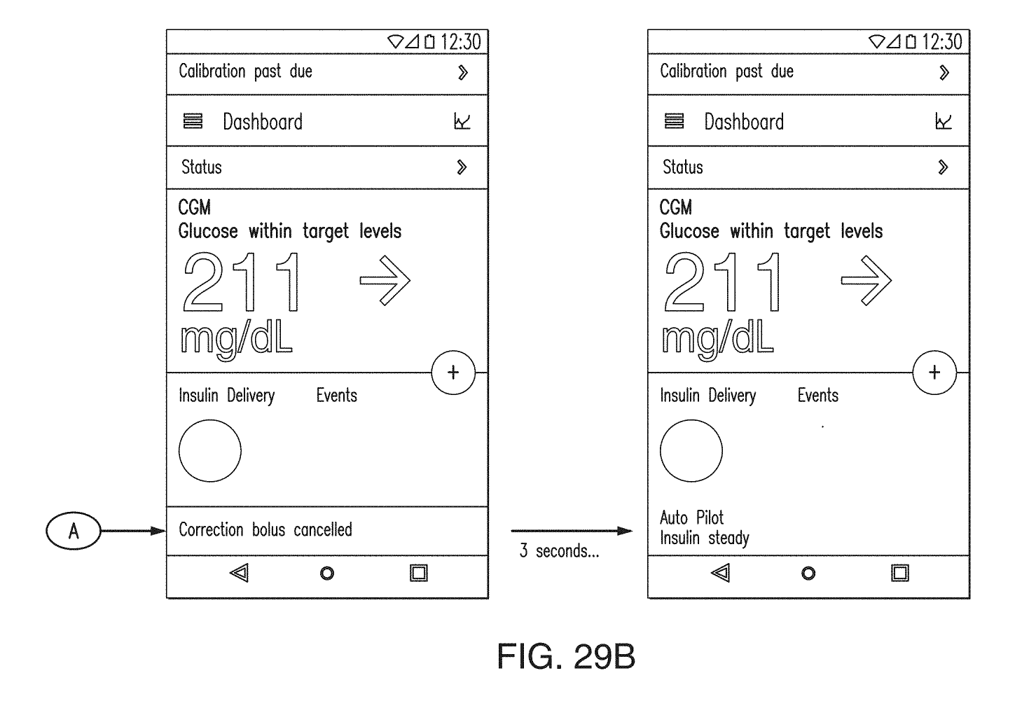

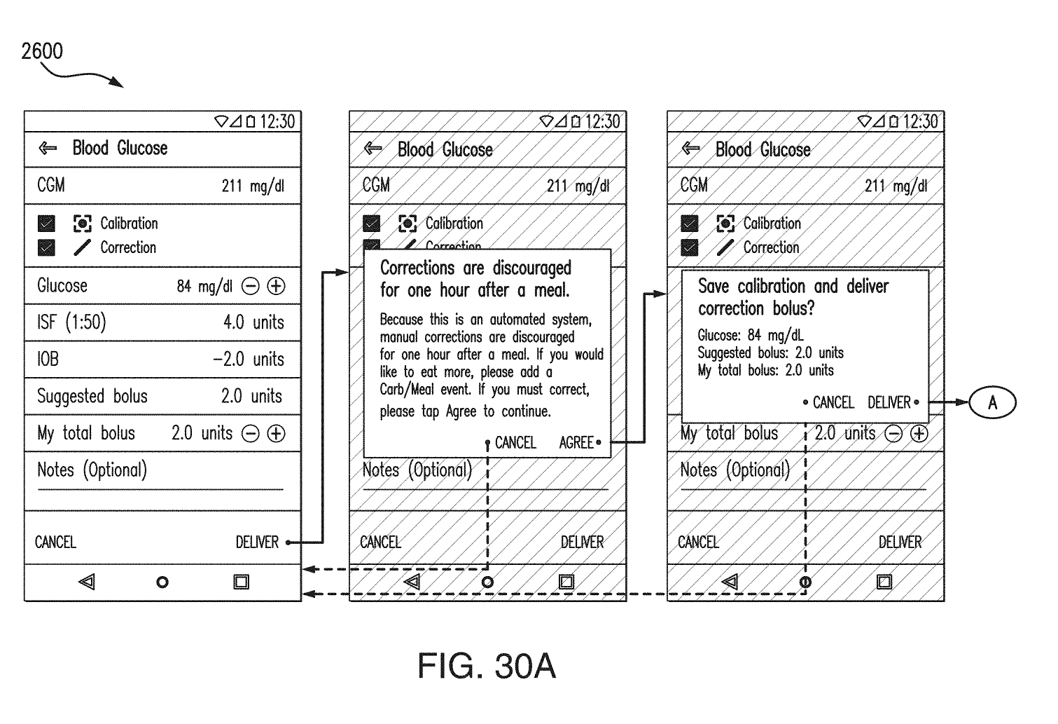

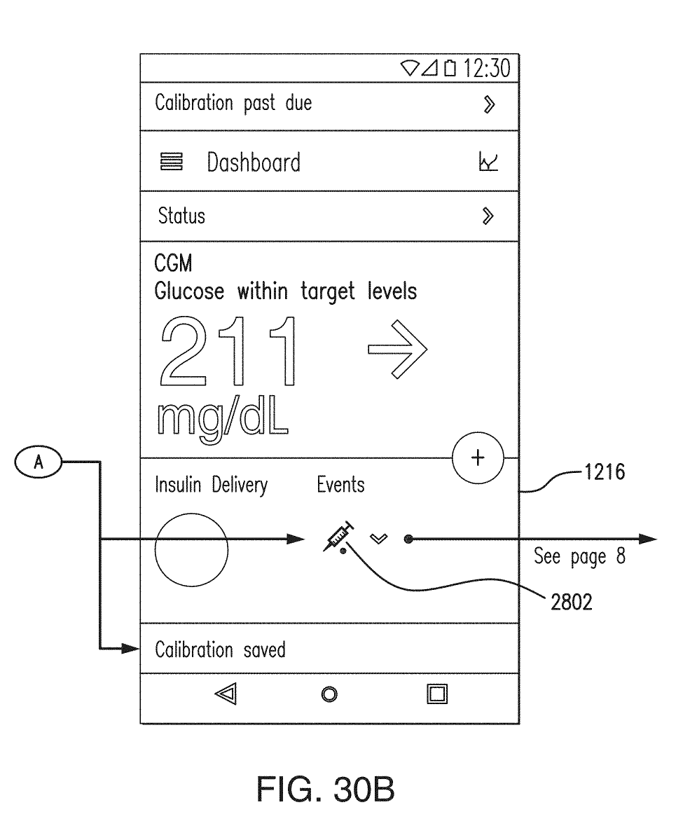

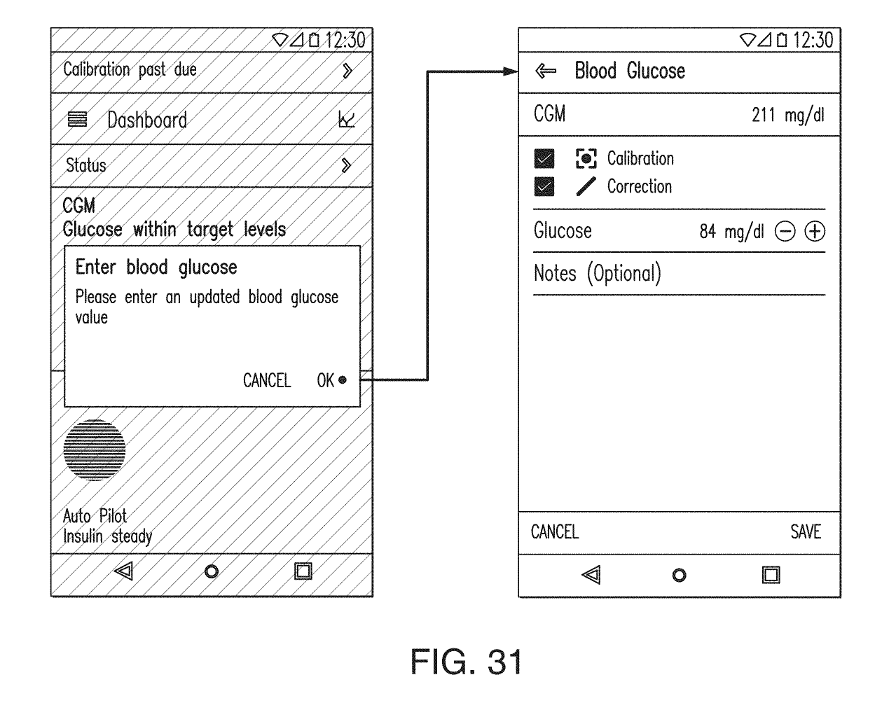

In some non-limiting embodiments, upon detection of contact made with the blood glucose event button 2502, the MMA 305 may configure the primary device 130 to present a blood glucose form 2600, as shown in FIGS. 26A and 26B. In some embodiments, a user may use the blood glucose form 2600 to save a blood glucose, as shown in FIGS. 26A and 26B. In some embodiments, as shown in FIG. 26B, the blood glucose saved notification may slide down and disappear after an amount of time (e.g., 3 seconds). In some embodiments, a user may use the blood glucose form 2600 to perform blood glucose calibration, as shown in FIGS. 27A and 27B. In some embodiments, as shown in FIG. 27B, the calibration saved notification may slide down and disappear after an amount of time (e.g., 3 seconds). In some embodiments, a user may use the blood glucose form 2600 to perform blood glucose correction, as shown in FIGS. 28A and 28B. In some embodiments, a standard bolus icon 2802 may be displayed in the events panel 1216 indicating a correction bolus as a result of the blood glucose correction, as shown in FIGS. 28A and 28B. In some embodiments, the correction bolus may be cancelled by initiating a cancellation process by selecting (e.g., touching) the standard bolus icon 2802, as shown in FIGS. 29A and 29B. In some embodiments, a user may use the blood glucose form 2600 to perform blood glucose calibration and correction, as shown in FIGS. 30A and 29B. In some embodiments, a standard bolus icon 2802 may be displayed in the events panel 1216 indicating a correction bolus as a result of the blood glucose calibration and correction, as shown in FIGS. 30A and 30B. In such embodiments, the correction bolus may be cancelled by initiating a cancellation process by selecting the standard bolus icon 2802, as shown in FIGS. 29A and 29B. In some embodiments, the AP functionality of the analyte monitoring and insulin delivery system 100 may prompt the user to enter blood glucose, as shown in FIG. 31.

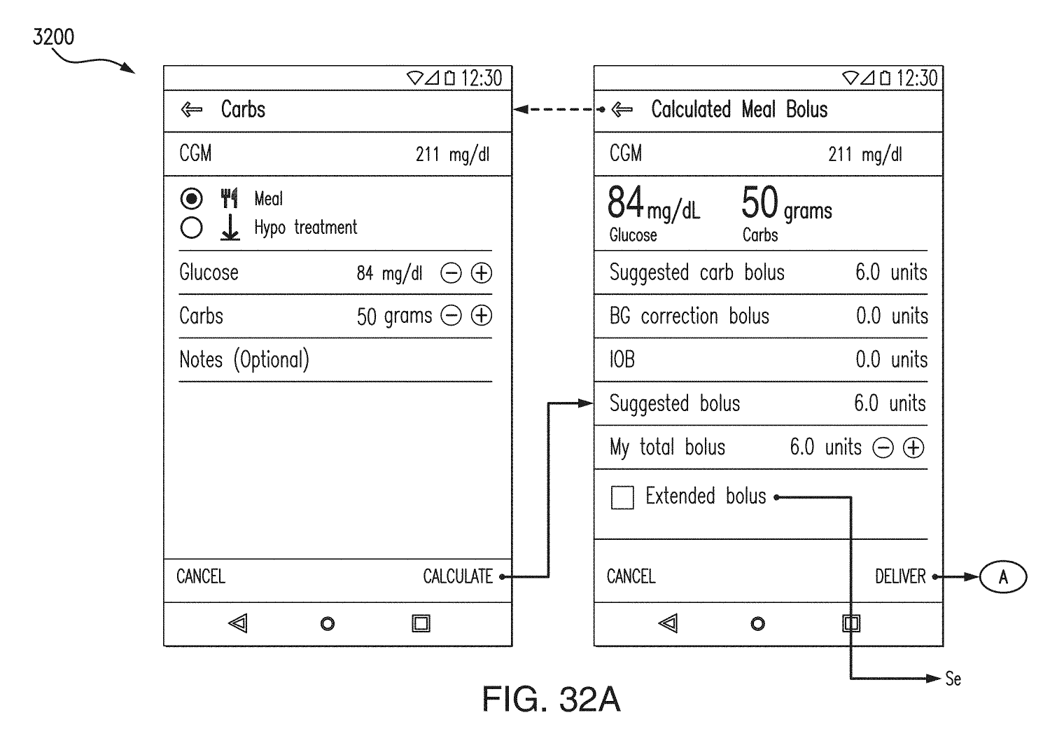

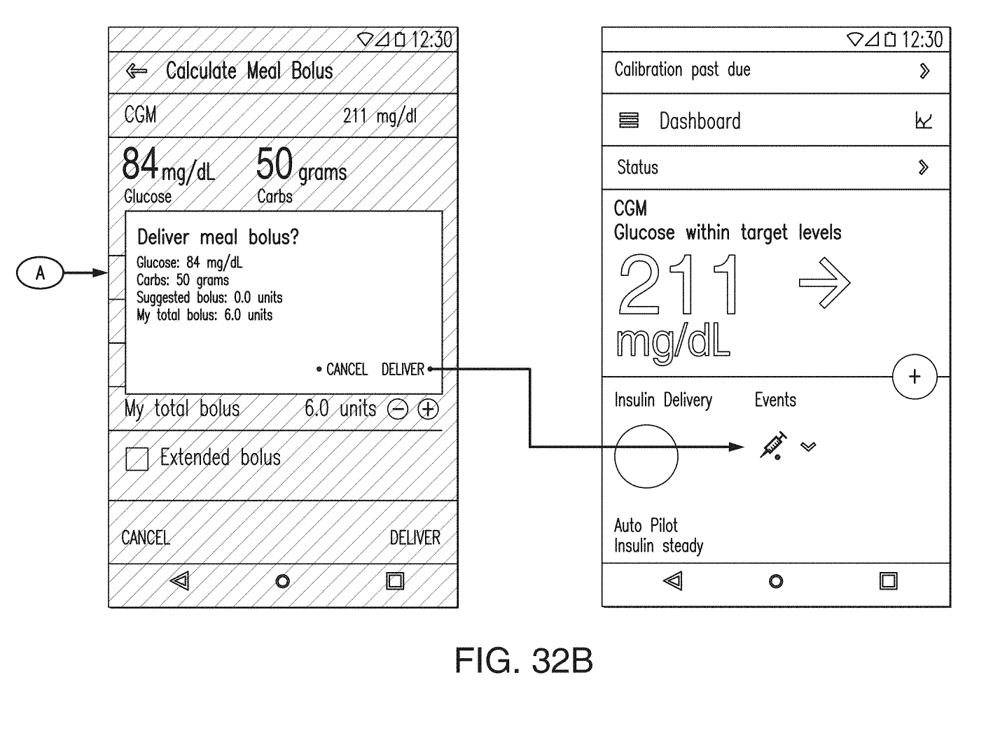

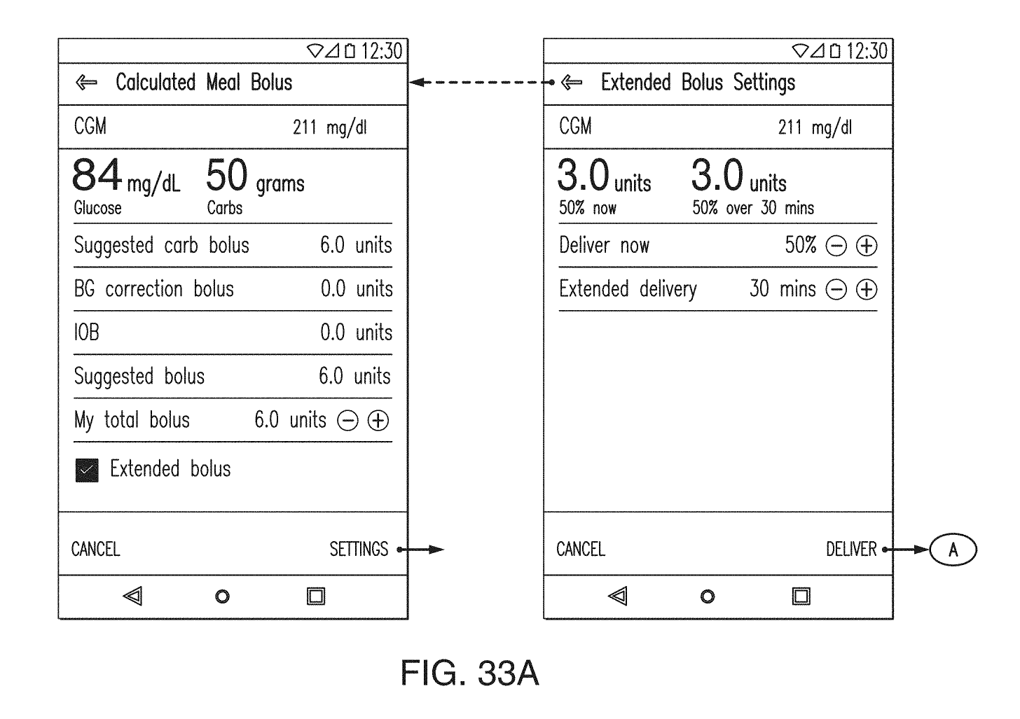

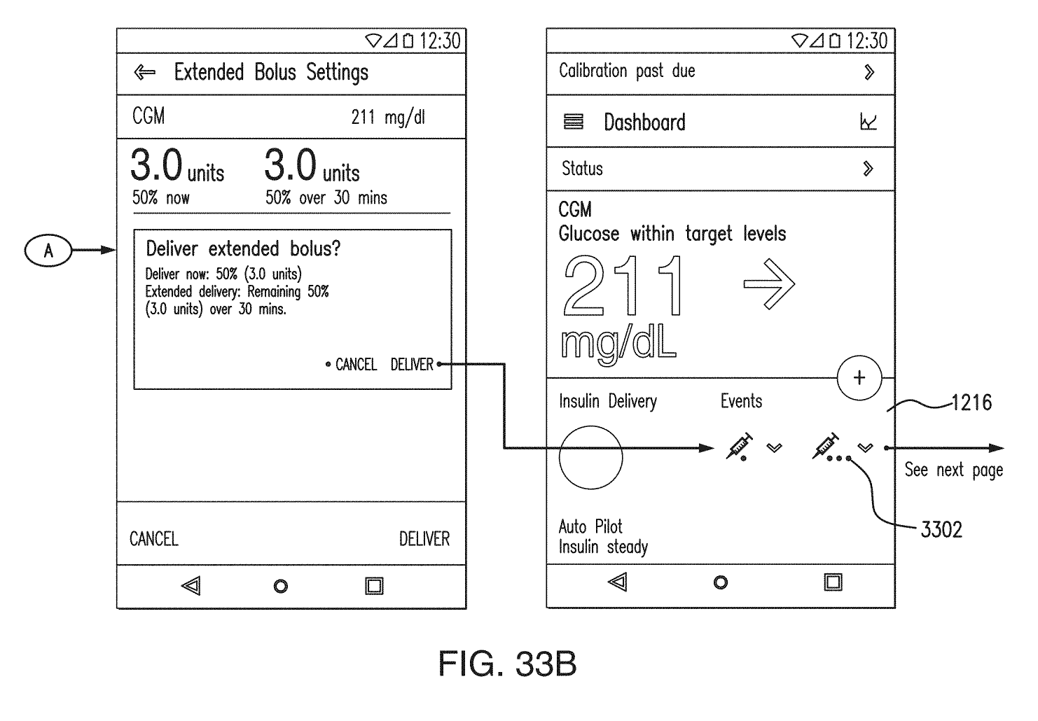

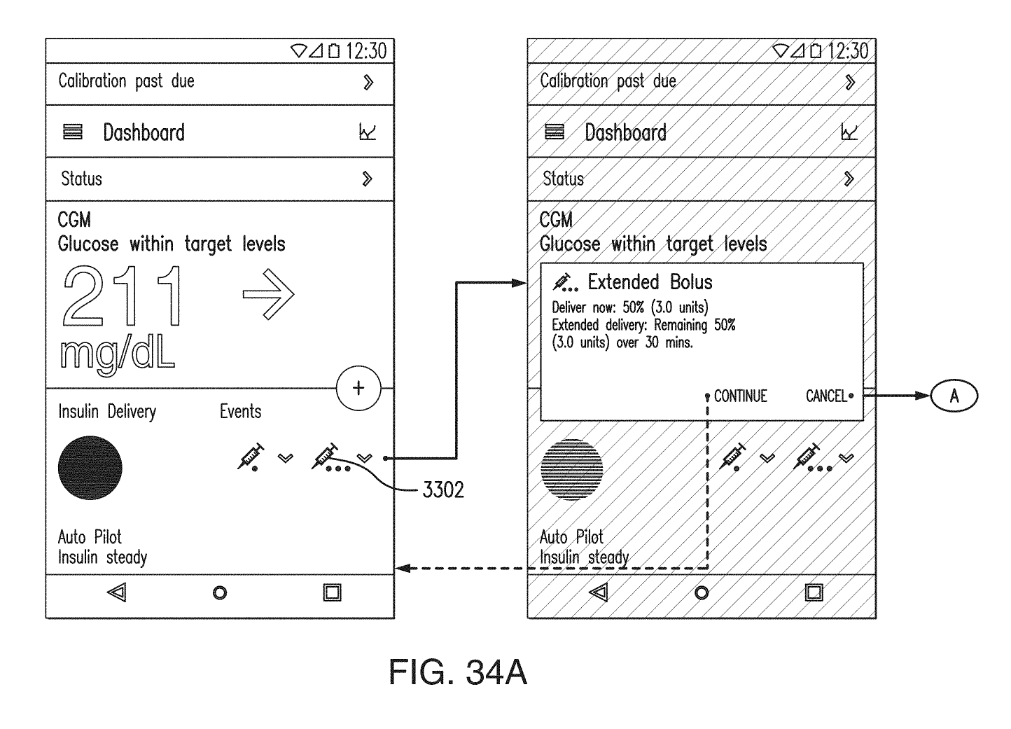

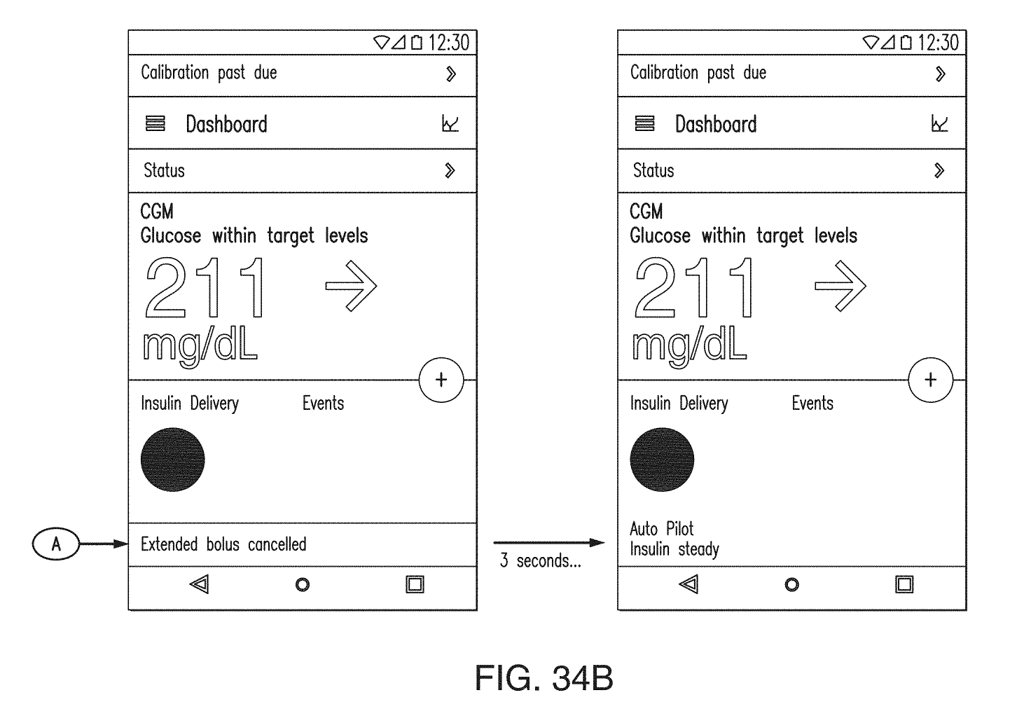

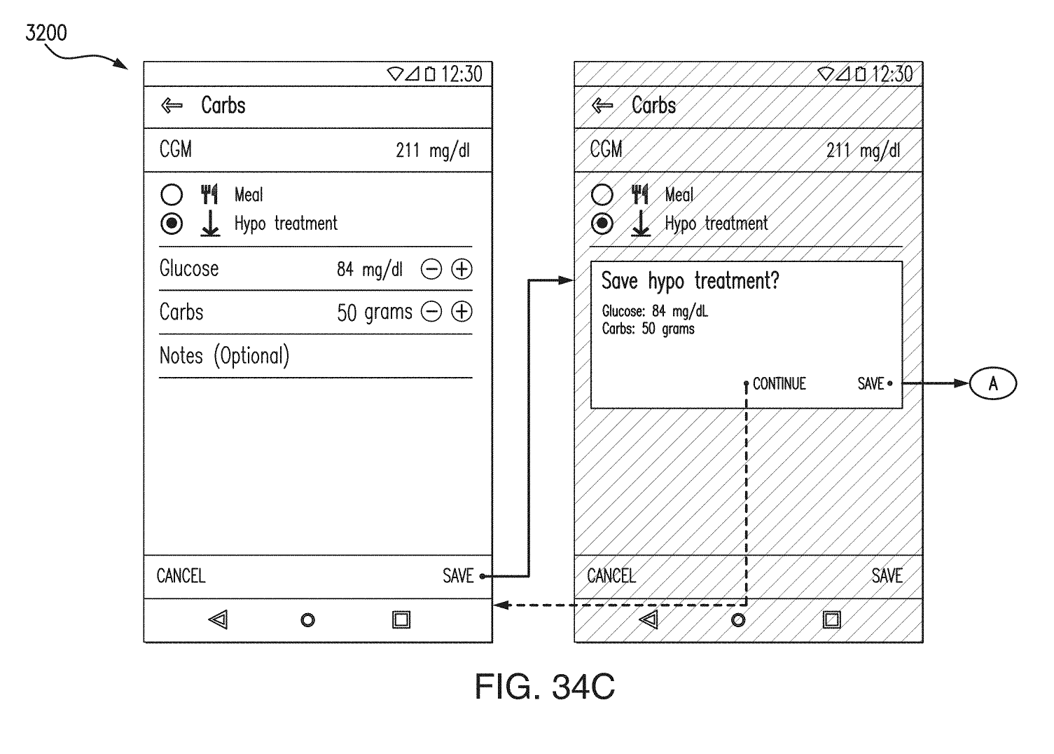

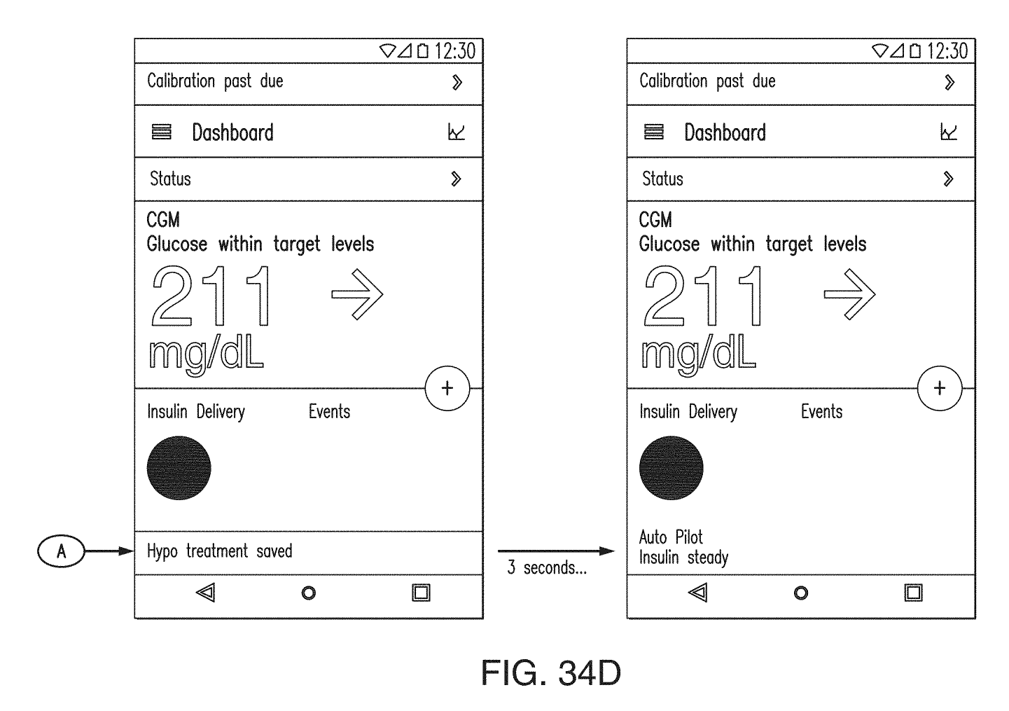

In some non-limiting embodiments, upon detection of contact made with the carbohydrates event button 2504, the MMA 305 may configure the primary device 130 to present a carbohydrates form 3200, as shown in FIGS. 32A and 32B. In some embodiments, a user may calculate and deliver a meal bolus using the carbohydrates form 3200, as shown in FIGS. 32A and 32B. In some embodiments, a user may calculate and deliver an extended bolus, as shown in FIGS. 33A and 33B. In some embodiments, an extended bolus icon 2802 may be displayed in the events panel 1216 indicating an extended bolus, as shown in FIGS. 33A and 33B. In such embodiments, the extended bolus may be cancelled by initiating a cancellation process by selecting the extended bolus icon 3302, as shown in FIGS. 34A and 34B. In some embodiments, as shown in FIG. 34B, the extended bolus canceled notification may slide down and disappear after an amount of time (e.g., 3 seconds). In some embodiments, a user may initiate and deliver a hypo treatment using the carbohydrates form 3200, as shown in FIGS. 34C and 34D. In some embodiments, as shown in FIG. 34B, the hypo treatment saved notification may slide down and disappear after an amount of time (e.g., 3 seconds).

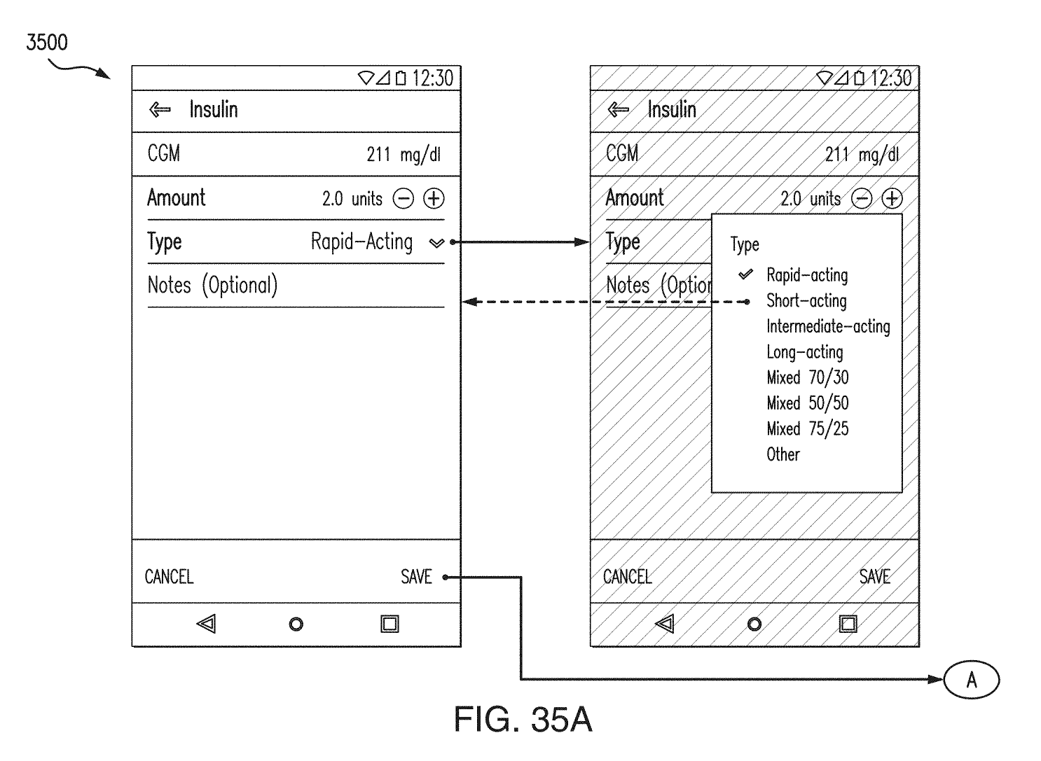

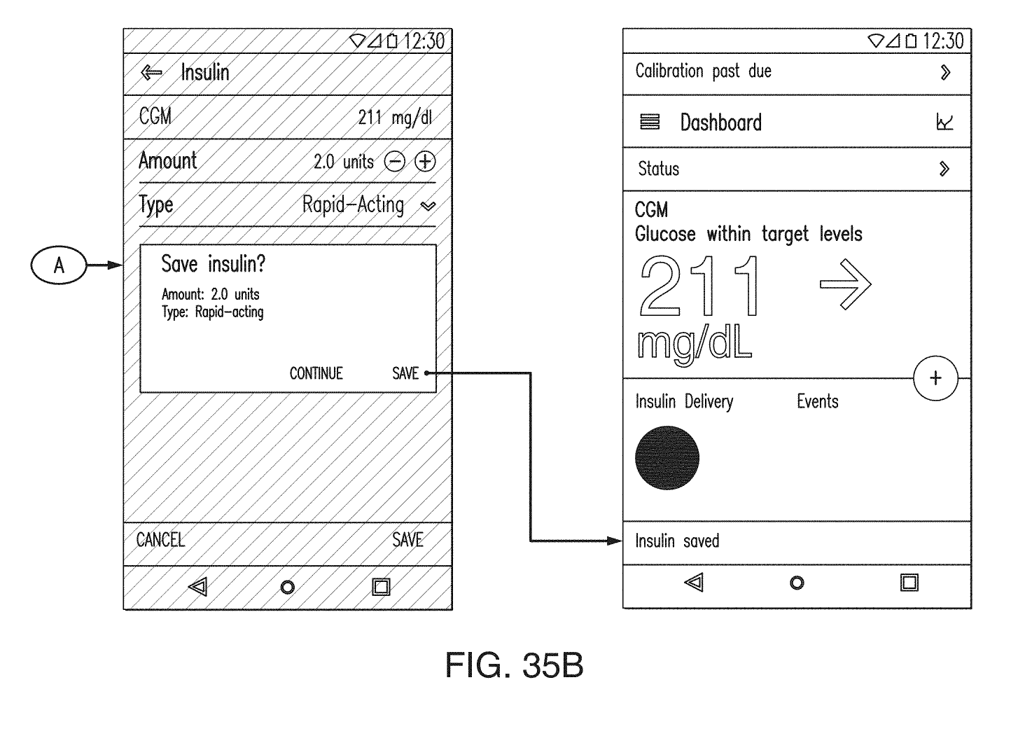

In some non-limiting embodiments, upon detection of contact made with the insulin event button 2506, the MMA 305 may configure the primary device 130 to present an insulin form 3500, as shown in FIGS. 35A and 35B. In some embodiments, an insulin amount may be saved using the insulin form 3500, as shown in FIGS. 35A and 35B.

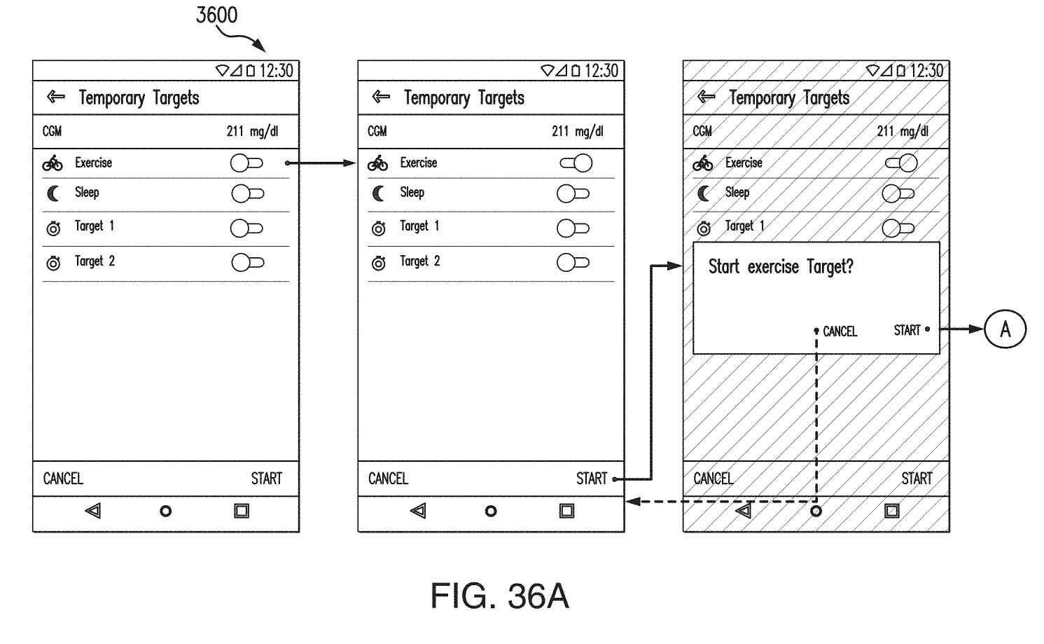

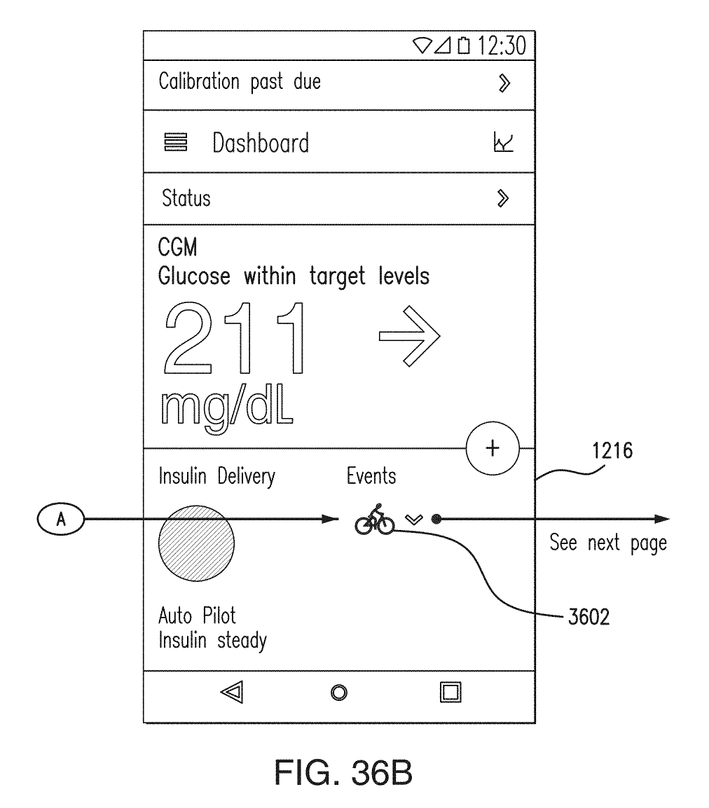

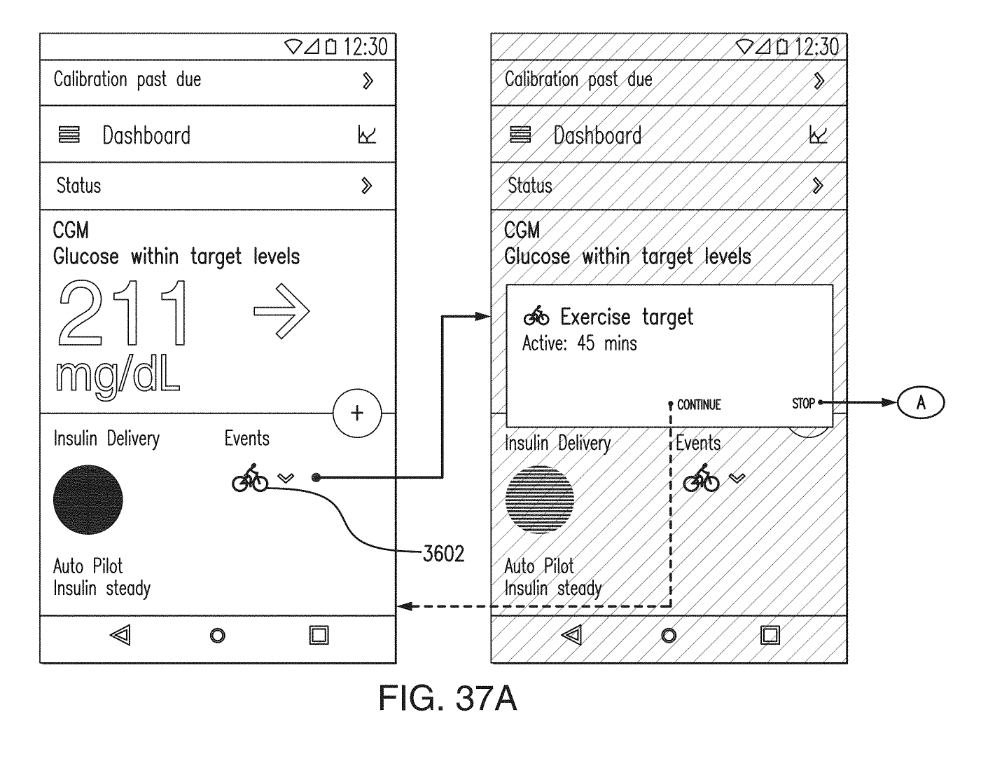

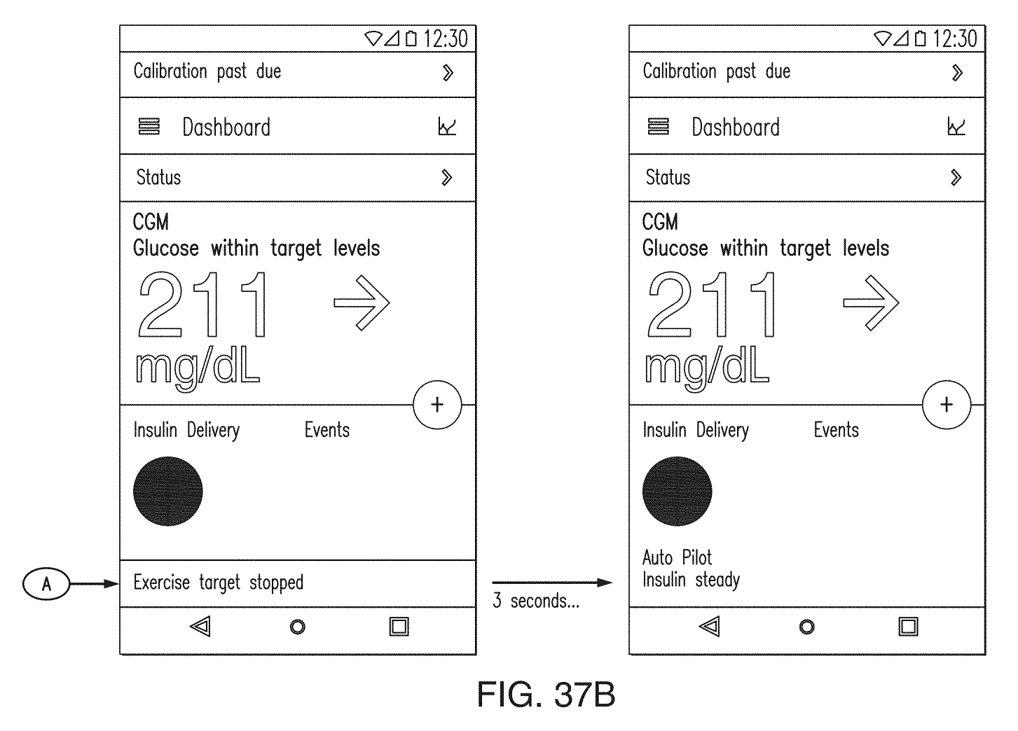

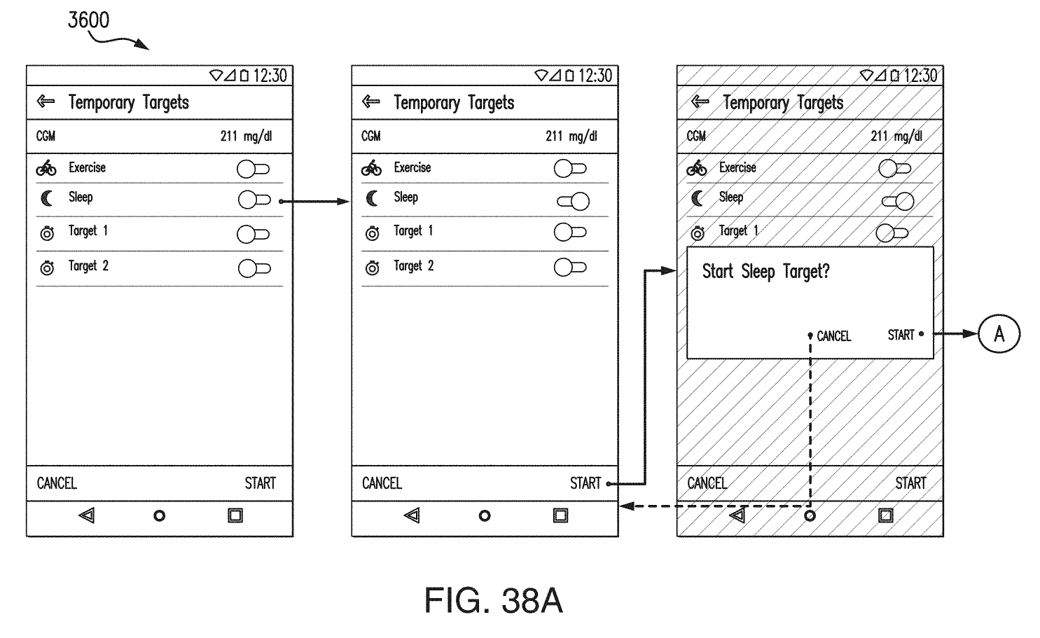

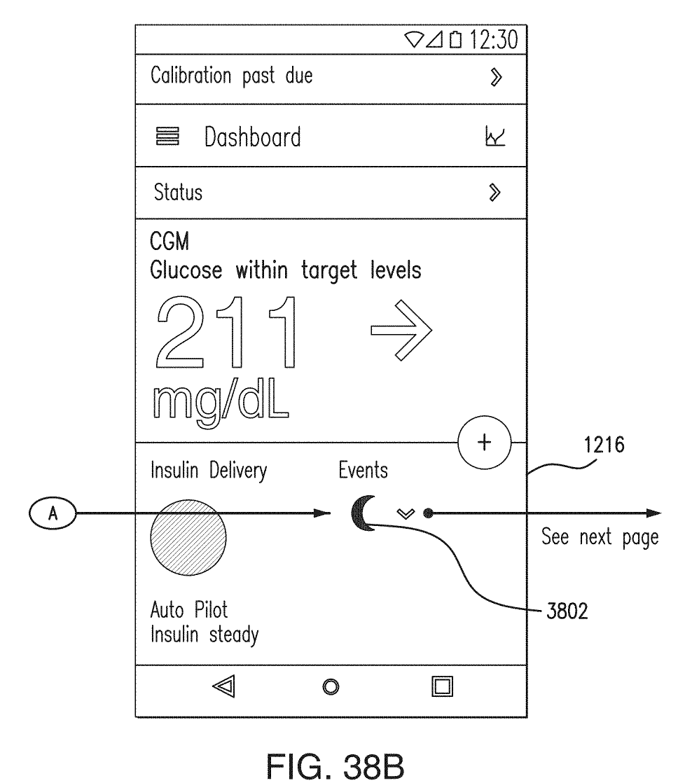

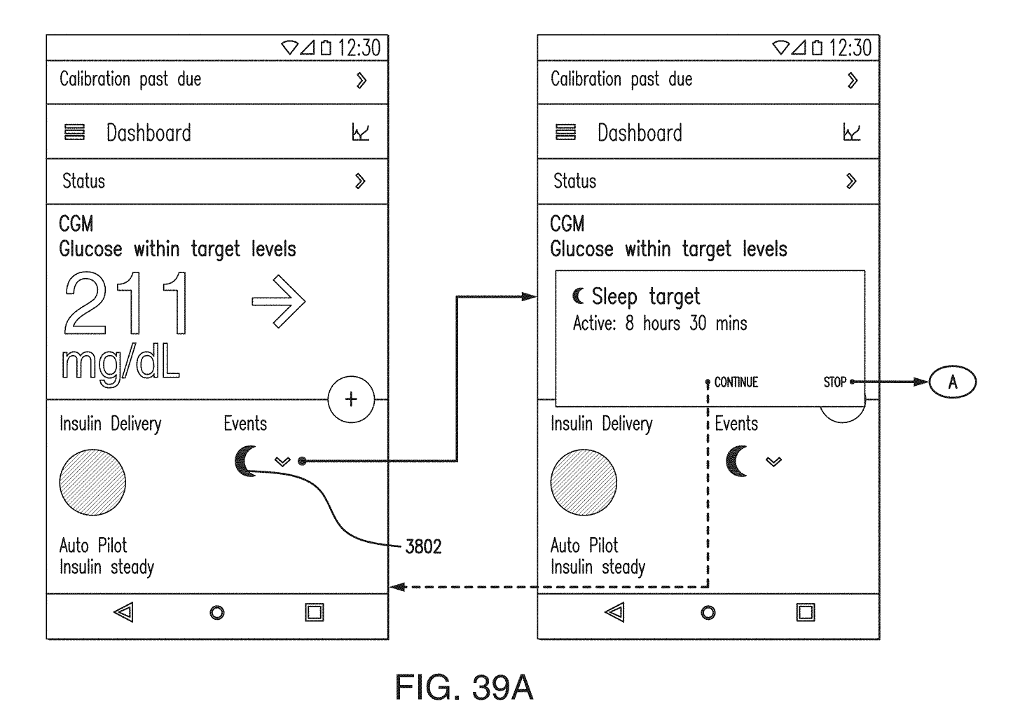

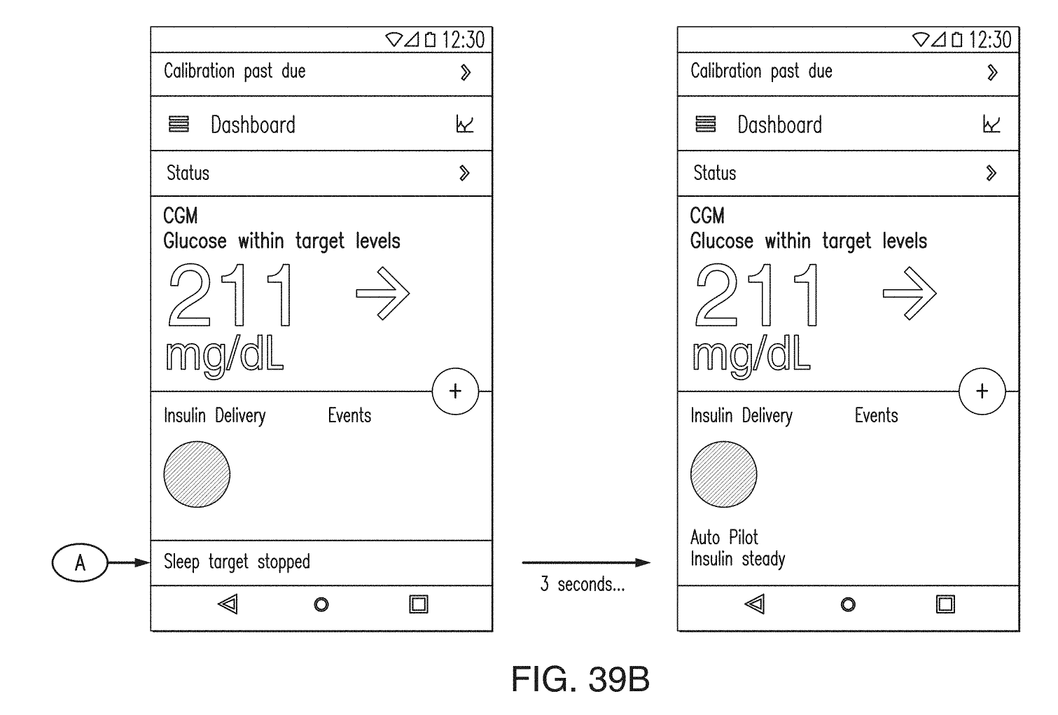

In some non-limiting embodiments, upon detection of contact made with the temporary targets button 2508, the MMA 305 may configure the primary device 130 to present a temporary targets form 3600, as shown in FIGS. 36A and 36B. In some embodiments, an exercise target may be established using the temporary targets form 3600, as shown in FIGS. 36A and 36B. In some embodiments, an exercise icon 3602 may be displayed in the events panel 1216 indicating an established exercise target, as shown in FIGS. 36A and 36B. In such embodiments, the exercise target may be cancelled by initiating a cancellation process by selecting the exercise icon 3602, as shown in FIGS. 37A and 37B. In some embodiments, a sleep target may be established using the temporary targets form 3600, as shown in FIGS. 38A and 38B. In some embodiments, a sleep icon 3802 may be displayed in the events panel 1216 indicating an established sleep target, as shown in FIGS. 38A and 38B. In such embodiments, the sleep target may be cancelled by initiating a cancellation process by selecting the sleep icon 3802, as shown in FIGS. 39A and 39B. In some embodiments, as shown in FIG. 39B, the sleep target stopped notification may slide down and disappear after an amount of time (e.g., 3 seconds).

FIG. 40 shows the trend graph screen 4000, according to some embodiments. In some embodiments, the trend graph screen 4000 may comprise at least one or more of the device operating system status bar 1202, the message bar, 1204, a title bar 4016, a navigation bar 4002, time markers 4004, axis 4006, a target range 4008, a glucose line 4010, a basal line 4012, events indications 4014, and the device operating system navigation bar 1214.

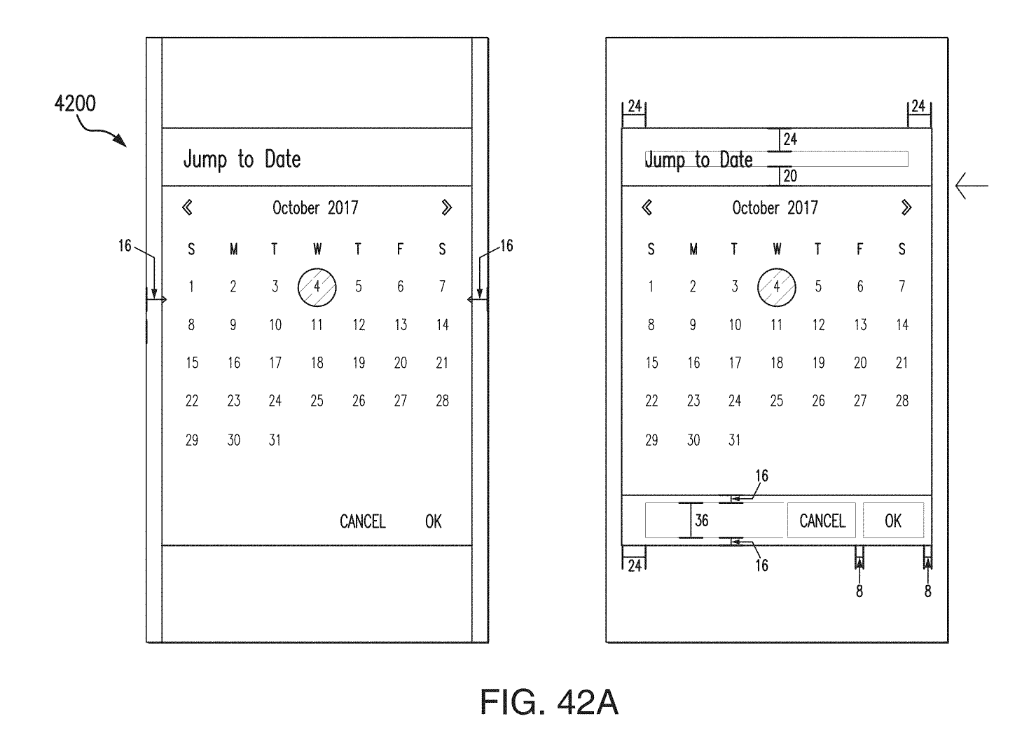

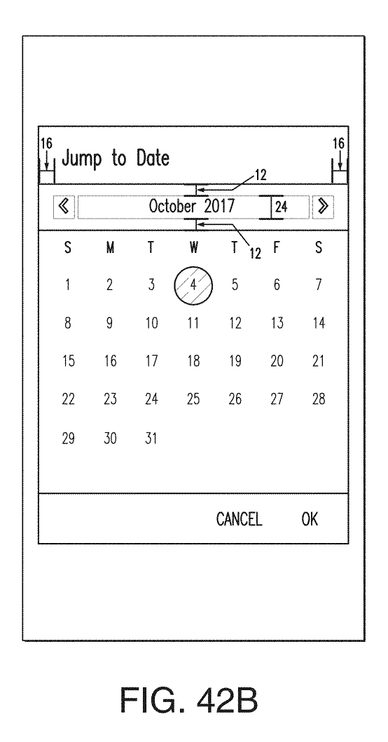

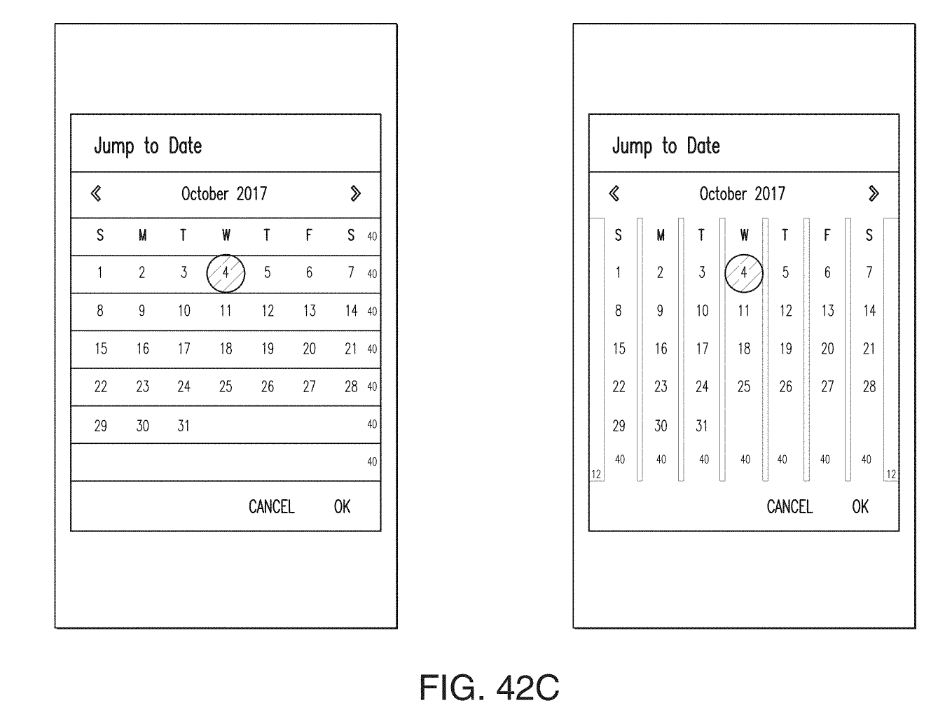

In some embodiments, the title bar 4016 may display an indication that the displayed screen is the trend graph screen 4000 and a calendar icon 4102. In some embodiments, selecting the calendar icon 4102 may open a date picker calendar 4200, as shown in FIG. 42A. In some embodiments, the date picker calendar 4200 may comprise a navigation bar, as shown in FIG. 42B. In some embodiments, date picker calendar 4200 may comprise a calendar grid, as shown in FIG. 42C.

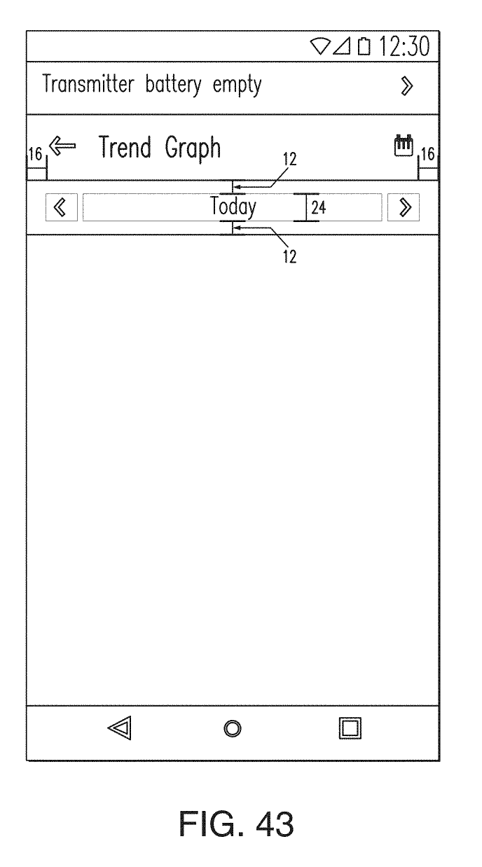

In some embodiments, the navigation bar 4002 may be displayed underneath the title bar 4016, as shown in FIG. 43.

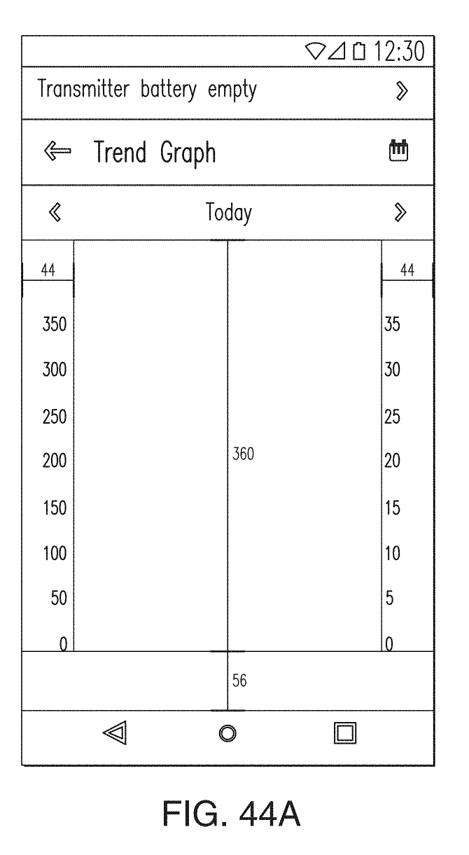

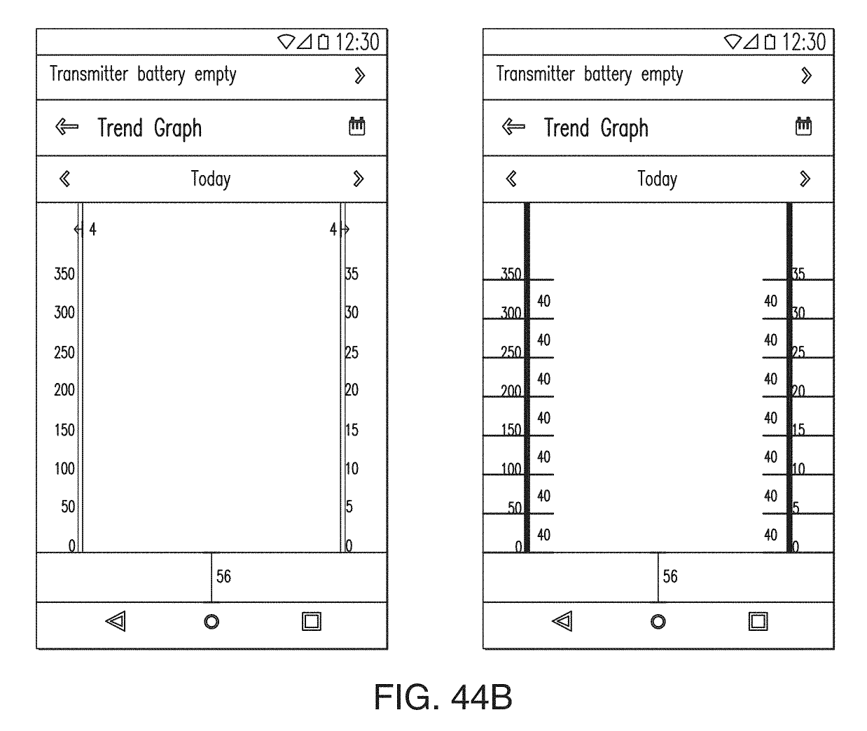

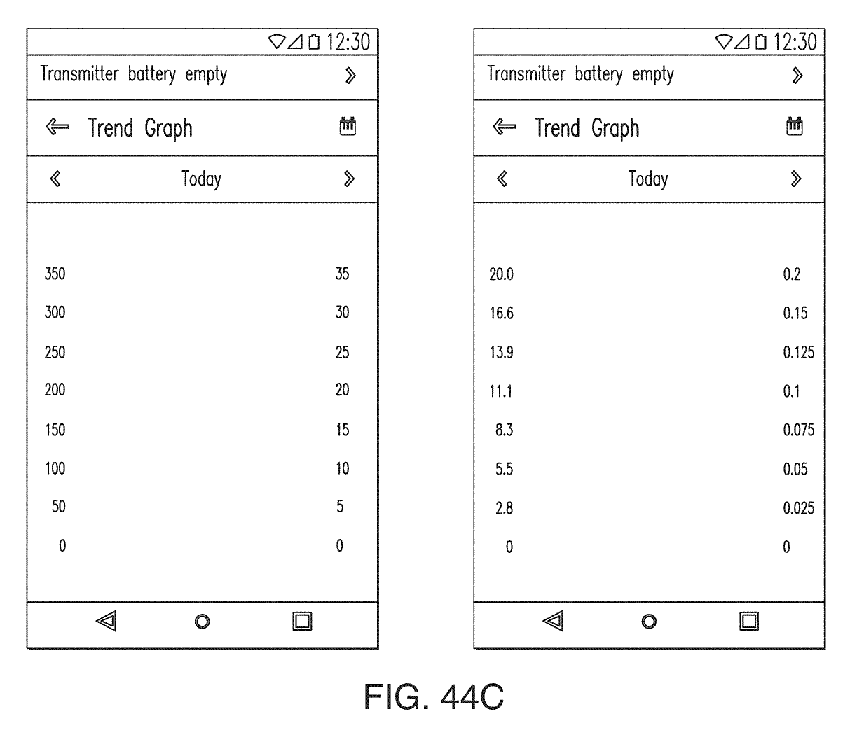

In some embodiments, the axis 4006 may be displayed as shown in FIGS. 44A-44C. In some embodiments, as shown in FIG. 44A, the GUI 215 may display two different axes, such as, for example and without limitation, a glucose axis (mg/dl or mmol/L) and a basal insulin axis (mg/dl or mmol/L).

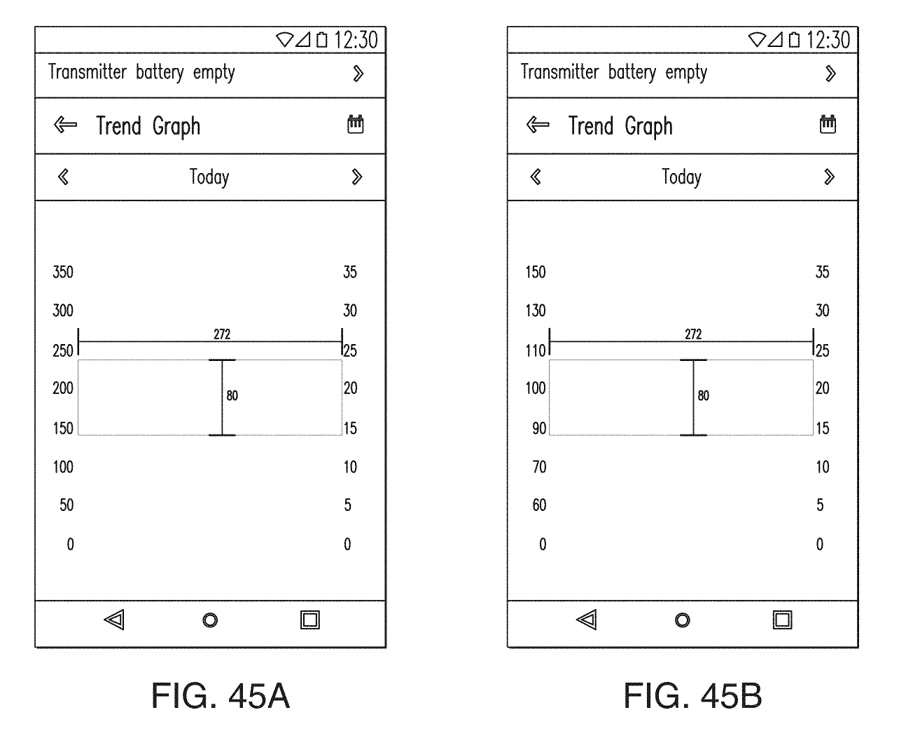

In some embodiments, the target range 4008 may be displayed (e.g., using a colored field, such as, for example and without limitation, a green field) as shown in FIGS. 45A and 45B. In some non-limiting embodiments, a user may specify the settings for target range.

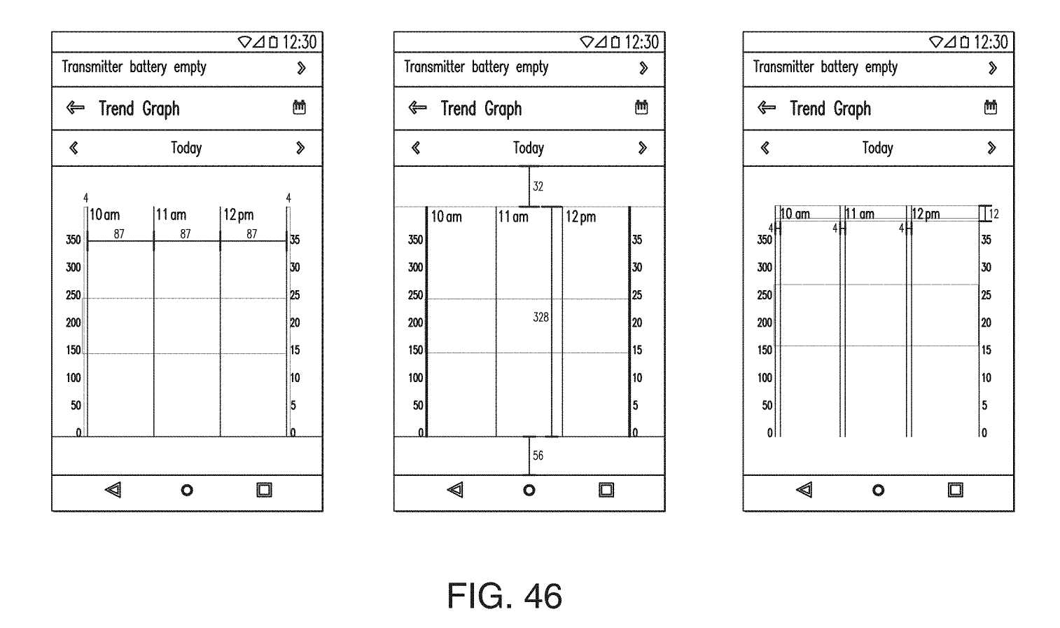

In some embodiments, the time markers 4004 may be displayed as shown in FIGS. 46A-46C.

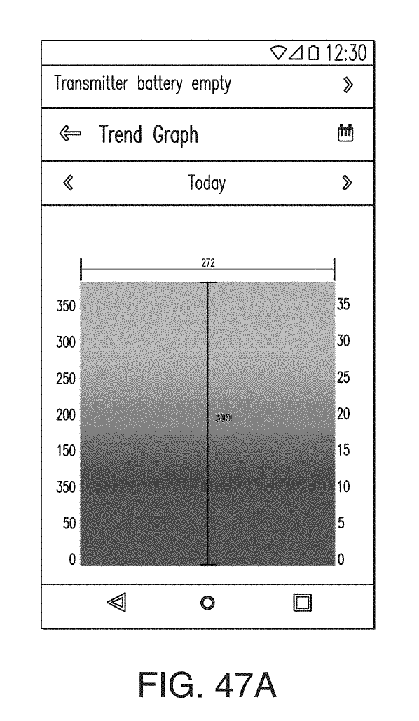

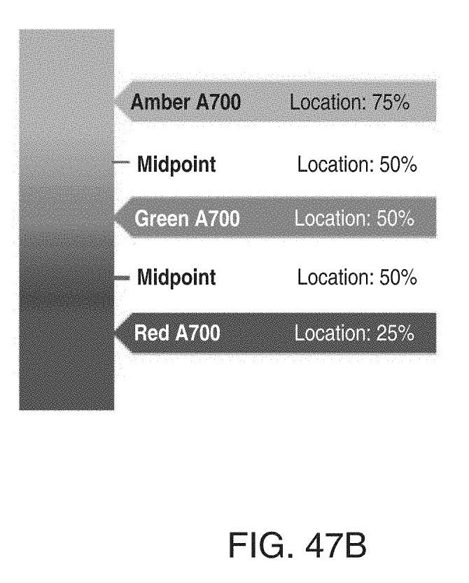

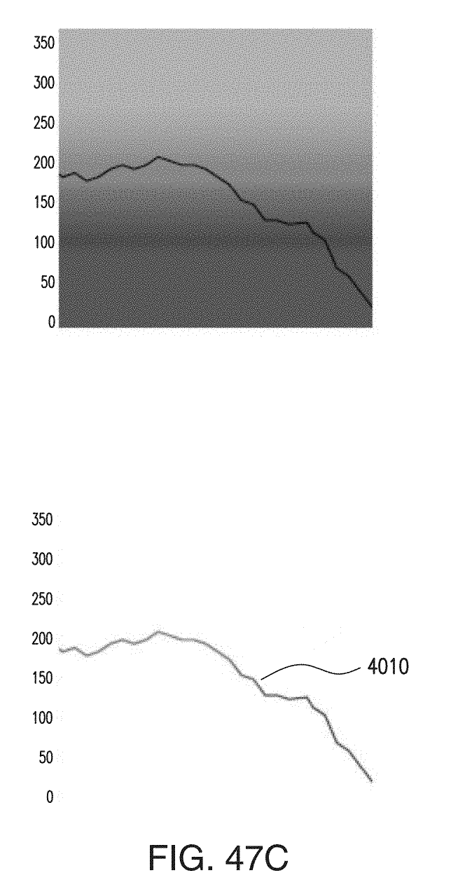

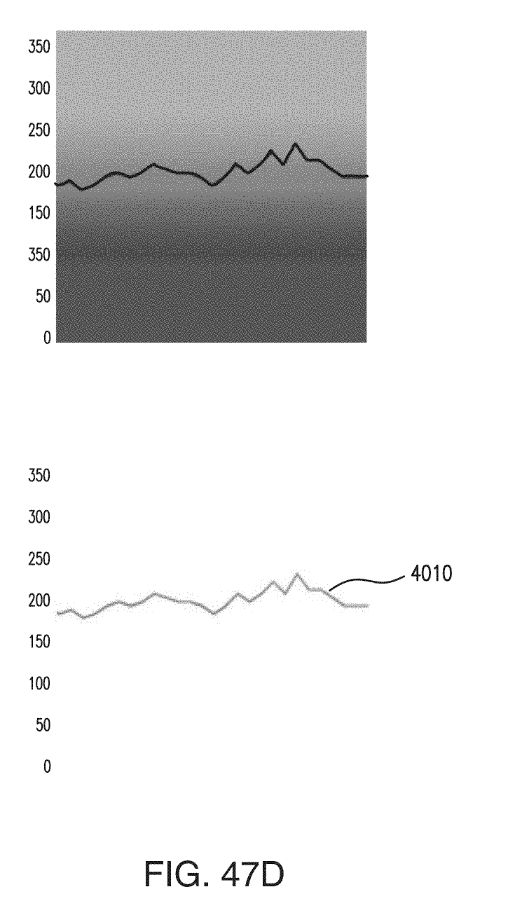

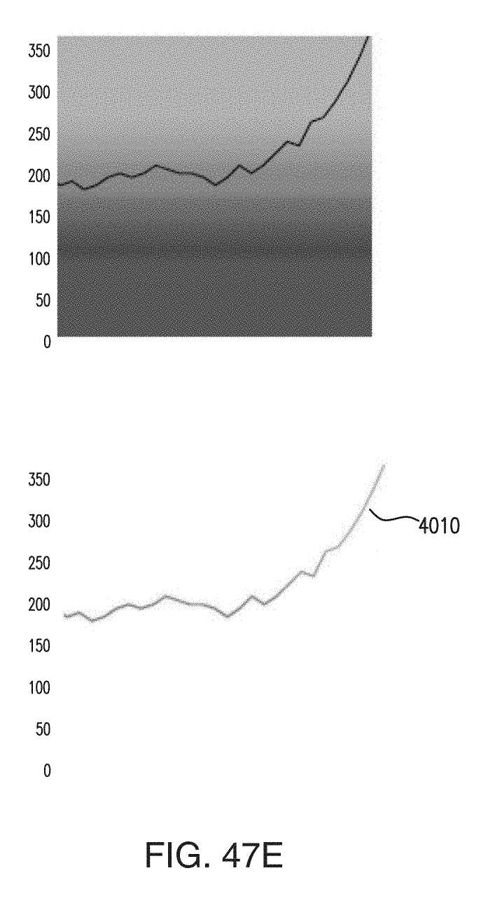

In some embodiments, the glucose line 4010 may be displayed as shown in FIGS. 47A-47E. In some non-limiting embodiments, different portions of the glucose line 4010 may be shown in different colors to indicate whether that portion of the glucose line 4010 is within a target range (e.g., a user-specified target range), above the target range, or below the target range. In some non-limiting embodiments, green may be used to indicate portions of the glucose line 4010 that are within the target range, yellow may be used to indicate that the glucose line 4010 is above the target range, and red may be used to indicate that the glucose line 4010 is below the target range. In some non-limiting embodiments, the color of the glucose line 4010 may change gradually, as shown in FIGS. 47C-47E. In some non-limiting embodiments, the gradual color change effect may be achieved by having a gradient base layer, as shown in FIGS. 47A and 47B, and then masking the glucose line 4010 above the gradient base layer, as shown in FIGS. 47C-47E. FIG. 47C shows non-limiting example of a glucose line 4010 that falls from the target range into a low glucose range, FIG. 47D shows non-limiting example of a glucose line 4010 that is steady within the target range, and FIG. 47E shows non-limiting example of a glucose line 4010 that rises from the target range into a high glucose range.

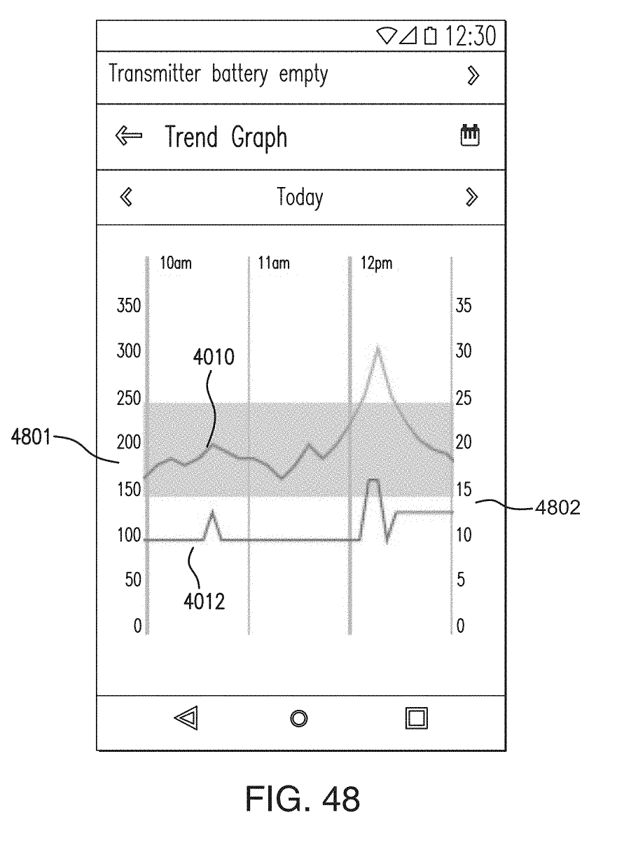

In some embodiments, the glucose line 4010 and the basal line 4012 may be displayed as shown in FIG. 48. In some embodiments, the glucose line 4010 and the basal line 4012 may be shown relative to a glucose axis 4801 and a basal axis 4802, respectively.

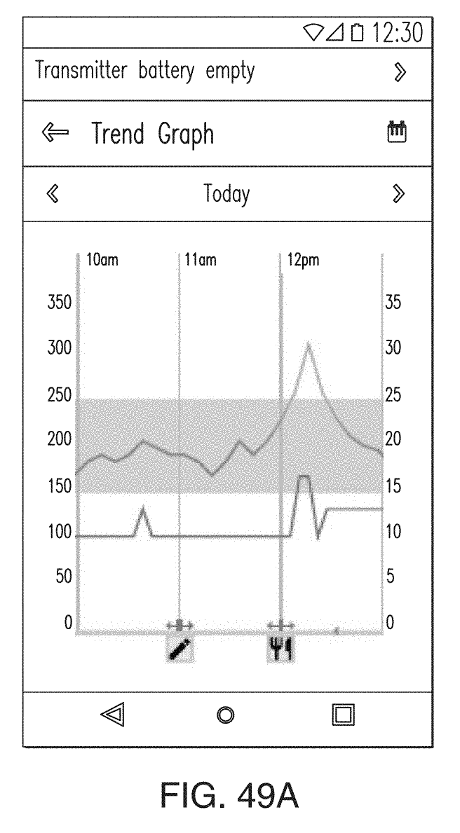

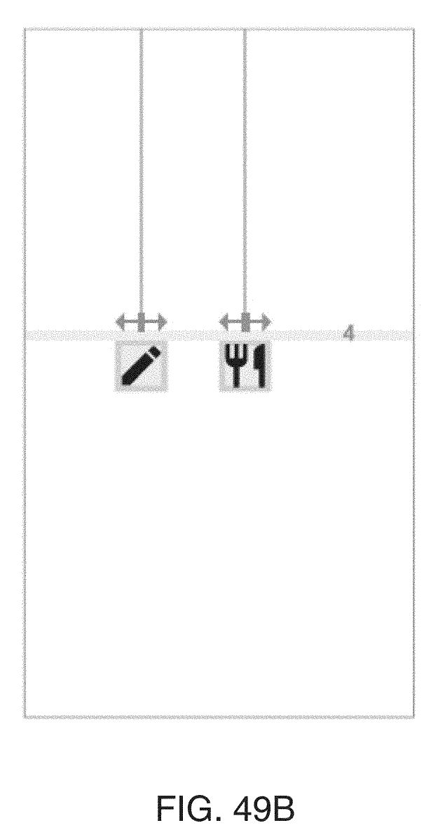

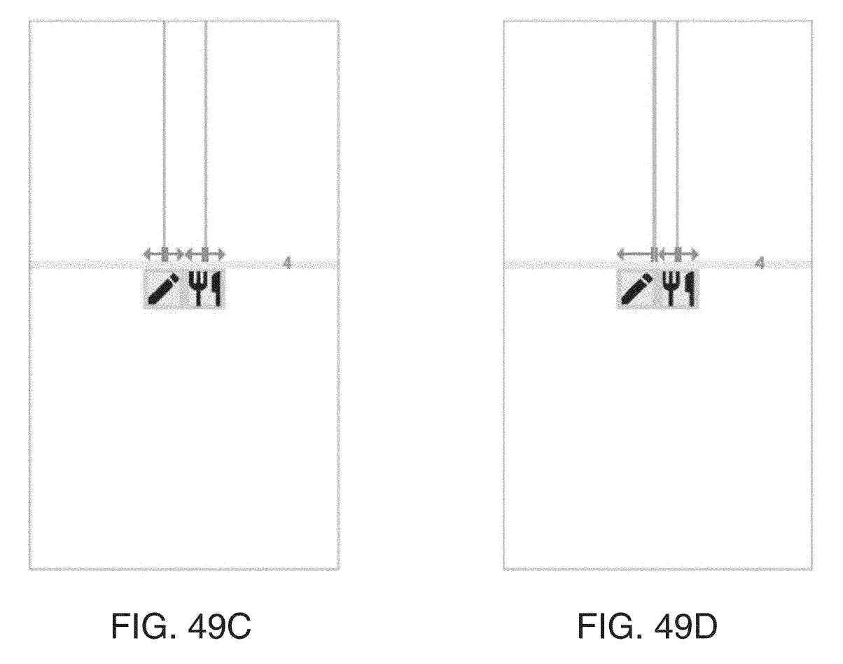

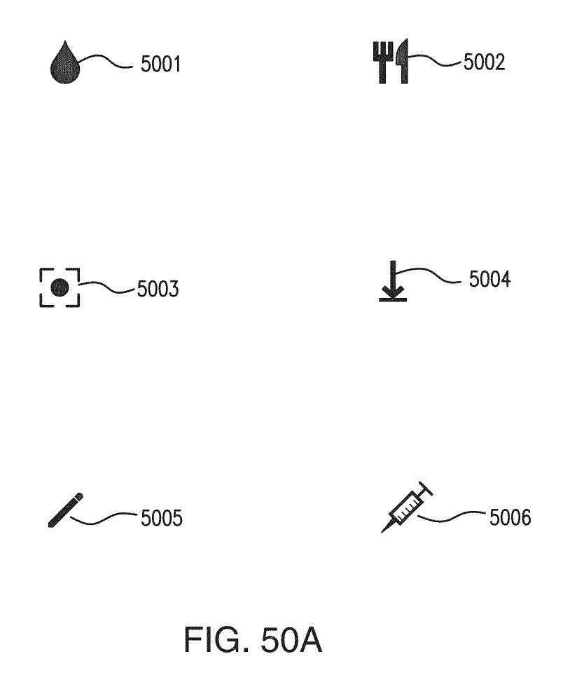

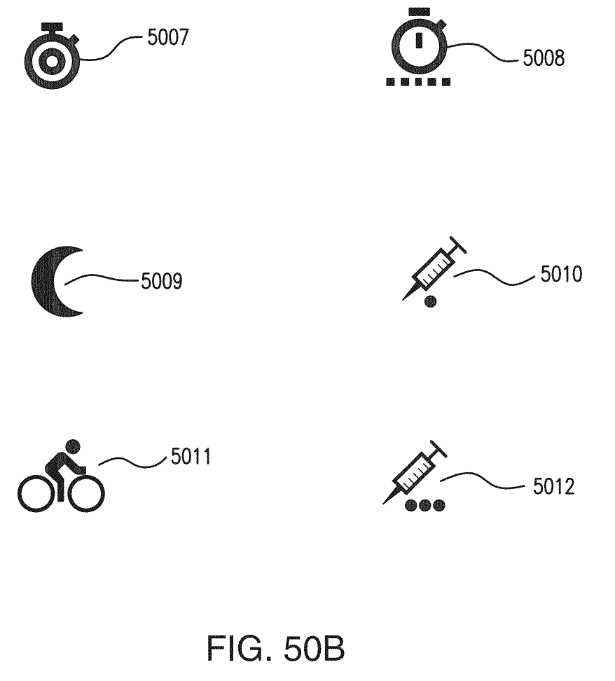

In some embodiments, the events indications 4014 may be displayed as shown in FIGS. 49A-49D. As shown in FIGS. 49A-49D, the event indications 4014 may comprise at least one or more event icons indicating an event. In some embodiments, as shown in FIG. 49A-49C, the event icons may be wider than the time marker lines. In some non-limiting embodiments, as shown in FIG. 49B, events spaced apart by a sufficient amount of time may be centered horizontally with respect to the time marker. In some non-limiting embodiments, as shown in FIGS. 49C and 49D, when events occur within a short time span, the event icons may be displayed right next to each other (e.g., with no padding). In some embodiments, as shown in FIG. 49D, when events occur at closer time intervals than the layout will allow, the event icons may be displayed right next to each other, but the event icons may not be centered horizontally with respect to the time marker. Some non-limiting exemplary event icons are shown in FIGS. 50A and 50B, according to some embodiments. As shown in FIG. 50A, the event icons may include one or more of a blood glucose event icon 5001, a meal event icon 1502, a calibration event icon 1503, a hype event icon 1504, a correction event 1505, and an insulin event 1506. As shown in FIG. 50B, the event icons may include one or more of a generic target event icon 1507, a temporary basal event icon 1508, a sleep target event icon 1509, a standard bolus event icon 1510, an exercise target event icon 511, and an extended bolus event icon 1512.

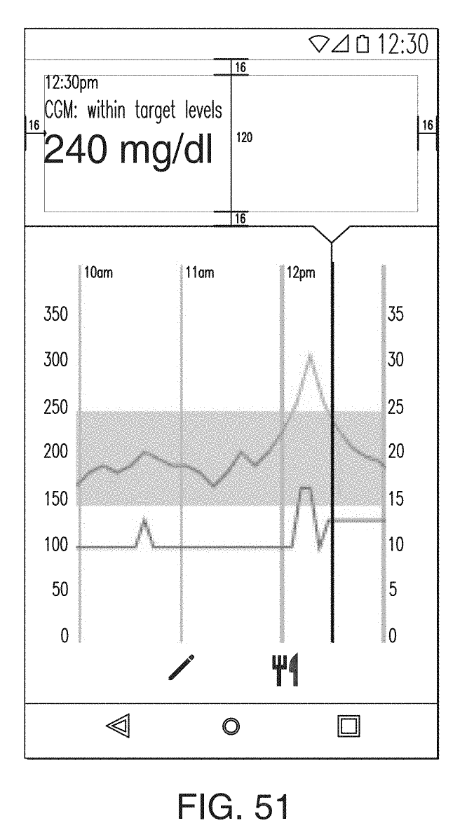

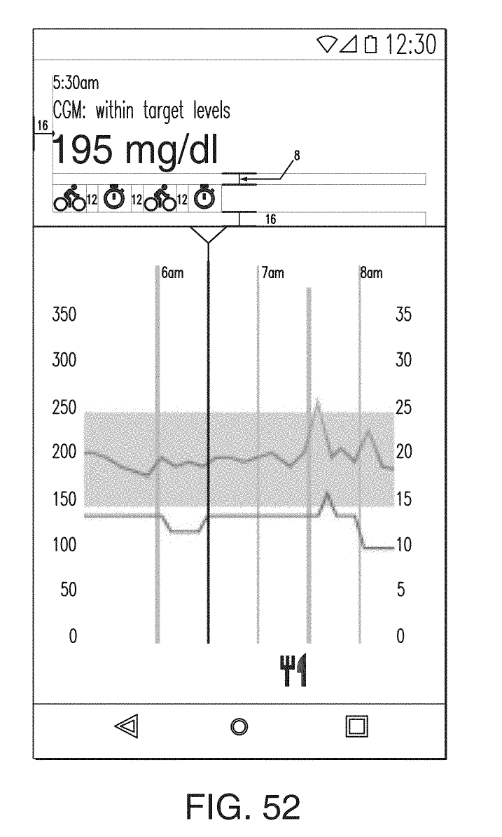

In some embodiments, pop over layers may be displayed as shown in FIG. 51. In some embodiments, event icons may be displayed in the pop over layers as shown in FIG. 52.

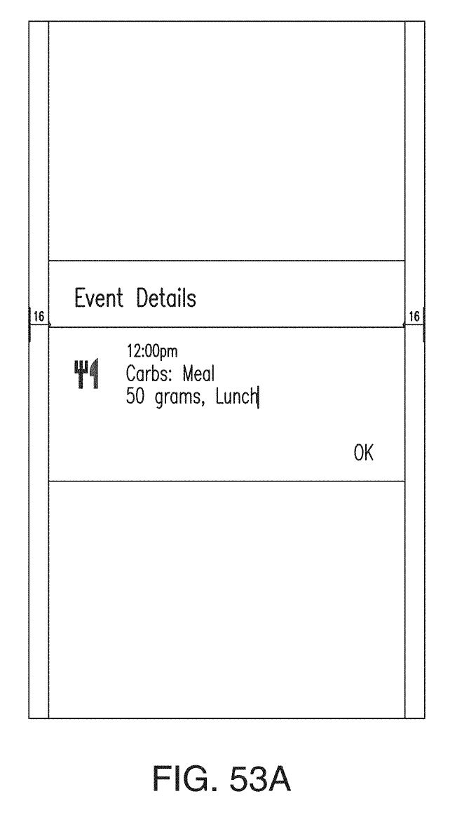

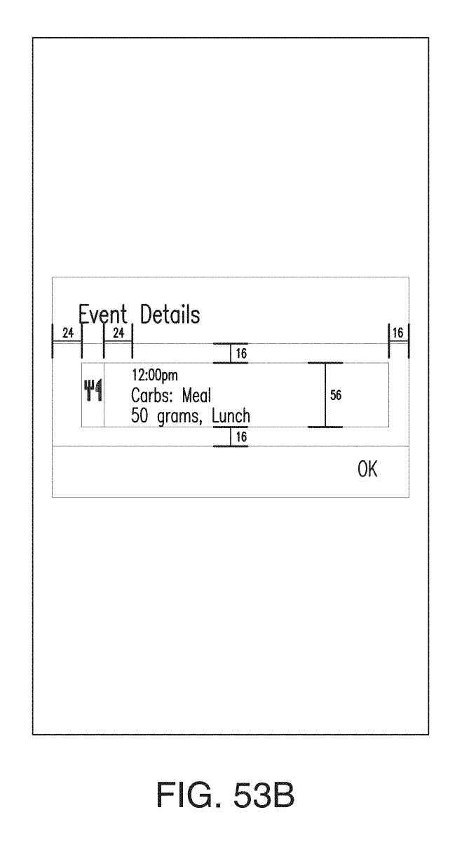

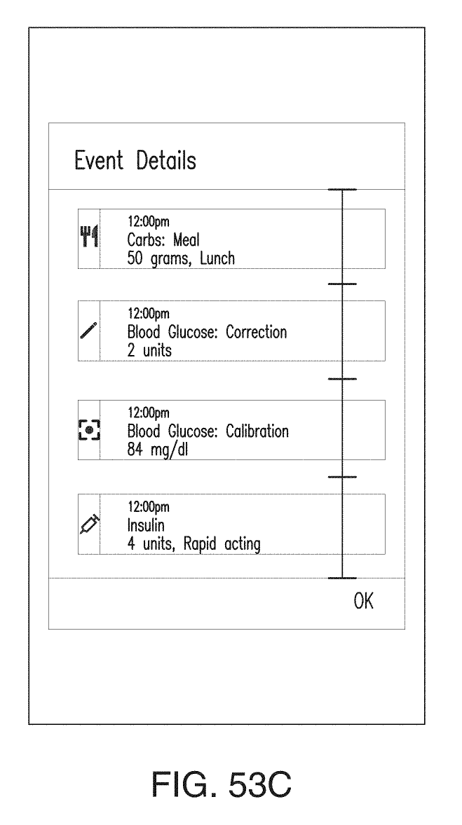

As shown in FIGS. 53A and 53B, dialog windows may be displayed when a user selects (e.g., touches or taps) an event icon shown in the events indications 4014, according to some embodiments. In some embodiments, the dialog windows may display details (e.g., event icon, timestamp, event type/name, and/or value/label) about a selected event. In some embodiments, as shown in FIG. 53C, if there are multiple events, a stack of event rows may displayed on top of one another in the dialog window, and a dialog window showing the details of multiple events may be taller than a dialog window showing the details of a single event.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. For example, in some alternative embodiments, one or more of the primary and secondary display devices may be a medical device such as, for example and without limitation, a blood glucose meter, an insulin pump, or a combination thereof. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

What is claimed is:

1. An analyte monitoring and insulin delivery system comprising:
an analyte sensor including an analyte indicator that produces one or more detectable properties based on an amount or concentration of an analyte in proximity to the analyte indicator, wherein the analyte sensor is configured to convey sensor data indicative of a measurement of one or more of the detectable properties;
a transceiver configured to:
receive the sensor data conveyed from the analyte sensor,
calculate an analyte level using at least the received sensor data, and
convey the analyte level;
an insulin pump; and
a display device configured to:
receive the analyte level conveyed from the transceiver,
calculate, based at least on the received analyte level, an adjusted delivery rate for the insulin pump,
convey the adjusted delivery rate to the insulin pump,
display, via a user interface, a first display comprising the received analyte level and the adjusted delivery rate, and
display, via the user interface, a second display comprising: (i) a sensor icon and a sensor status indicator indicating an operational status of the analyte sensor, (ii) a transceiver icon and a transceiver status indicator indicating an operational status of the transceiver, and (iii) a pump icon and an insulin pump status indicator indicating an operational status of the insulin pump;
wherein the insulin pump is configured to:
deliver insulin at a first delivery rate,
receive the adjusted delivery rate conveyed by the display device, and
increase, decrease, or maintain the first delivery rate based on the received adjusted delivery rate.

2. The analyte monitoring and insulin delivery system of claim 1, further comprising:
an analyte meter configured to:
receive the analyte level conveyed from the transceiver, and
display the received analyte level;
wherein the second display further comprises a meter icon and a meter status indicator indicating an operational status of the analyte meter.

3. The analyte monitoring and insulin delivery system of claim 1, wherein the display device is further configured to compare the adjusted delivery rate with the first delivery rate; and
the first display further comprises a delivery rate indication of whether the adjusted delivery rate is an increase, a decrease, or a continuation of the first delivery rate.

4. The analyte monitoring and insulin delivery system of claim 1, wherein:
the display device is further configured to compare the received analyte level with one or more previously stored analyte levels; and
the first display further comprises an indication of whether the received analyte level is an increase, a decrease, or a continuation of the one or more previously stored analyte levels.

5. The analyte monitoring and insulin delivery system of claim 1, wherein the first display further comprises an indication that the received analyte level is below, within, or above a predetermined range of analyte levels.

6. The analyte monitoring and insulin delivery system of claim 1, wherein the first display further comprises a message bar configured to display one or more messages containing information regarding the analyte monitoring and insulin delivery system.

7. The analyte monitoring and insulin delivery system of claim 1, wherein the first display further comprises an analyte trend graph icon, the display device is further configured to display a third display in response to a user selection of the of the analyte trend graph icon, and the third display comprises an analyte trend graph showing an analyte level line that indicates a change in analyte level over a period of time.

8. The analyte monitoring and insulin delivery system of claim 1, wherein the first display further comprises an events panel comprising one or more event icons.

9. The analyte monitoring and insulin delivery system of claim 1, wherein the second display further comprises an autopilot icon and an autopilot status indicator indicating an operational status of automatic adjustment of insulin delivery.

10. The analyte monitoring and insulin delivery system of claim 1, wherein the sensor, transceiver, and insulin pump status indicators indicate an operational status with one of a normal sign, a warning sign, a failure sign, and a status unknown sign.

11. The analyte monitoring and insulin delivery system of claim 1, wherein the second display comprises a visual map of the analyte monitoring and insulin delivery system, and the visual map includes at least the sensor icon, the sensor status indicator, the transceiver icon, the transceiver status indicator, the pump icon, and the insulin pump status indicator.

12. The analyte monitoring and insulin delivery system of claim 1, wherein one or more of the sensor icon, the transceiver icon, and the pump icon of the second display are selectable, and the second device is further configured to, in response to a selection of one of the icons, provide additional information about the one of the analyte sensor, the transceiver, and the insulin pump to which the selected icon corresponds.

13. An analyte monitoring and insulin delivery method comprising:
using a transceiver to receive sensor data from an analyte sensor, wherein the analyte sensor includes an analyte indicator that produces one or more detectable properties based on an amount or concentration of an analyte in proximity to the analyte indicator the analyte sensor is configured to convey the sensor data, and the sensor data is indicative of a measurement of one or more of the detectable properties;
using the transceiver to calculate an analyte level using at least the received sensor data;
using the transceiver to convey the analyte level;
using a display device to receive the analyte level conveyed from the transceiver;
using the display device to calculate, based at least on the received analyte level, an adjusted delivery rate for an insulin pump;
using the display device to convey the adjusted delivery rate to the insulin pump;
using the display device to display, via a user interface, a first display comprising the received analyte level and the adjusted delivery rate;
using the display device to display, via the user interface, a second display comprising: (i) a sensor icon and a sensor status indicator indicating an operational status of the analyte sensor, (ii) a transceiver icon and a transceiver status indicator indicating an operational status of the transceiver, and (iii) a pump icon and an insulin pump status indicator indicating an operational status of the insulin pump;
using the insulin pump to deliver insulin at a first delivery rate;
using the insulin pump to receive the adjusted delivery rate conveyed by the display device; and
using the insulin pump to increase, decrease, or maintain the first delivery rate based on the received adjusted delivery rate.

14. The analyte monitoring and insulin delivery method of claim 13, further comprising:
using an analyte meter to receive the analyte level conveyed from the transceiver; and
using the analyte meter display the received analyte level,
wherein the second display further comprises a meter icon and a meter status indicator indicating an operational status of the analyte meter.

15. The analyte monitoring and insulin delivery method of claim 13, further comprising using the display device to compare the adjusted delivery rate with the first delivery rate;
wherein the first display further comprises a delivery rate indication of whether the adjusted delivery rate is an increase, a decrease, or a continuation of the first delivery rate.

16. The analyte monitoring and insulin delivery method of claim 13, further comprising using the display device to compare the received analyte level with one or more previously stored analyte levels;
wherein the first display further comprises an indication of whether the received analyte level is an increase, a decrease or a continuation of the one or more previously stored analyte levels.

17. The analyte monitoring and insulin delivery method of claim 13, wherein the first display further comprises an indication that the received analyte level is below, within, or above a predetermined range of analyte levels.

18. The analyte monitoring and insulin delivery method of claim 13, wherein the first display further comprises a message bar configured to display one or more messages containing information regarding the analyte monitoring and insulin delivery system.

19. The analyte monitoring and insulin delivery method of claim 13, wherein the first display further comprises an analyte trend graph icon, the method further comprises displaying a third display in response to a user selection of the of the analyte trend graph icon, and the third display comprises an analyte trend graph showing an analyte level line that indicates a change in analyte level over a period of time.

20. The analyte monitoring and insulin delivery method of claim 13, wherein the first display further comprises an events panel comprising one or more event icons.

21. The analyte monitoring and insulin delivery method of claim 13, wherein the second display further comprises an autopilot icon and an autopilot status indicator indicating an operational status of automatic adjustment of insulin delivery.

22. The analyte monitoring and insulin delivery method of claim 13, wherein the sensor, transceiver, and insulin pump status indicators indicate an operational status with one of a normal sign, a warning sign, a failure sign, and a status unknown sign.

23. The analyte monitoring and insulin delivery method of claim 13, wherein the second display comprises a visual map of an analyte monitoring and insulin delivery system including at least the analyte sensor, transceiver, and insulin pump, and the visual map includes at least the sensor icon, the sensor status indicator, the transceiver icon, the transceiver status indicator, the pump icon, and the insulin pump status indicator.

24. The analyte monitoring and insulin delivery method of claim 13, wherein one or more of the sensor icon, the transceiver icon, and the pump icon of the second display are selectable, and the method further comprises, in response to a selection of one of the icons, providing additional information about the one of the analyte sensor, the transceiver, and the insulin pump to which the selected icon corresponds.
